(19) United States
(12) Patent Application Publication
(10) Pub. No.: US 2024/0150450 A1
(43) Pub. Date: May 9, 2024

(54) ANTI-AMYLOID BETA ANTIBODIES AND USES THEREOF

(71) Applicant: Eli Lilly and Company, Indianapolis, IN (US)

(72) Inventors: Mark Mintun, Indianapolis, IN (US); John Randall Sims, Zionsville, IN (US)

(21) Appl. No.: 18/549,803

(22) PCT Filed: Mar. 11, 2022
(86) PCT No.: PCT/US2022/019898

§ 371 Date: Sep. 8, 2023

Related U.S. Application Data
(60) Provisional application No. 63/192,271, filed on May 24, 2021.
(60) Provisional application No. 63/160,642, filed on Mar. 12, 2021.

(51) Int. Cl. C07K 16/18; A61P 25/28

(52) U.S. Cl. CPC C07K 16/18; A61P 25/28; C07K 2317/24

(57) ABSTRACT

The invention is directed to treatment or prevention of a disease characterized by deposition of Aβ in the brain using anti-Aβ antibodies. The diseases that can be treated or prevented include, e.g., Alzheimer's disease, Down's syndrome, and cerebral amyloid angiopathy. The invention is also related to, in some aspects, to selecting a human subject based on the tau burden in the human subjects' brain, who is responsive to treatment or prevention of a disease characterized by deposition of Aβ in the brain that includes administering anti-Aβ antibodies. The invention is also related to human subjects who have one or two alleles of APOE e4.

The present invention, in some aspects, relates to methods of prevention or treatment of a disease with anti-Aβ antibodies, wherein the disease is characterized by deposition of Amyloid Beta (Aβ) in a human subject. The diseases that can be treated or prevented using antibodies, dosing regimens, or methods disclosed herein include, e.g., Alzheimer's disease (AD), Down's syndrome, and cerebral amyloid angiopathy (CAA). One aspect of the present invention is related to treating or preventing the disease characterized by deposition of Aβ in human subjects, wherein the human subjects are selected for treatment or prevention based on their tau level/burden in the whole brain (e.g., global tau), in portions of the brain (e.g., in different lobes of the brain), and/or the presence of one or two alleles of APOE e4 in the patient's genome.

A cure for AD is one of the most significant unmet need of society. Accumulation of amyloid-β (Aβ) peptide in the form of brain amyloid deposits is an early and essential event in Alzheimer's disease (AD), leading to neurodegeneration and consequently the onset of clinical symptoms: cognitive and functional impairment (Selkoe, “The Origins of Alzheimer Disease: A is for Amyloid,” JAMA 283:1615-7 (2000); Hardy et al., “The Amyloid Hypothesis of Alzheimer's Disease: Progress and Problems on the Road to Therapeutics,” Science 297:353-6 (2002); Masters et al., “Alzheimer's Disease,” Nat. Rev. Dis. Primers 1:15056 (2015); and Selkoe et al., “The Amyloid Hypothesis of Alzheimer's Disease at 25 years,” EMBO Mol. Med. 8:595-608 (2016)).

Amyloid Beta (Aβ) is formed by the proteolytic cleavage of a larger glycoprotein called amyloid precursor protein (APP). APP is an integral membrane protein expressed in many tissue, but especially in neuron synapses. APP is cleaved by γ-secretase to release the Aβ peptide, which encompasses a group of peptides ranging in size from 37-49 amino acid residues. Aβ monomers aggregate into various types of higher order structures including oligomers, and protofibrils and amyloid fibrils. Amyloid oligomers are soluble and may spread throughout the brain, while amyloid fibrils are larger and insoluble and can further aggregate to form amyloid deposits or plaques. The amyloid deposits found in human patients include a heterogeneous mixture of Aβ peptides, some of which include N-terminal truncations and further may include N-terminal modifications such as an N-terminal pyroglutamate residue (pGlu).

A role for amyloid deposits in driving disease progression is supported by study of uncommon genetic variants that either increase or decrease Aβ deposition (Fleisher et al., “Associations Between Biomarkers and Age in the Presenilin 1 E280A Autosomal Dominant Alzheimer Disease Kindred: A Cross-sectional Study,” JAMA Neurol 72:316-24 (2015); Jonsson et al., “A Mutation in APP Protects Against Alzheimer's Disease and Age-related Cognitive Decline,” Nature 488:96-9 (2012)). In addition, the presence of amyloid deposits early in the disease increases the likelihood of progression of mild cognitive impairment (MCI) to AD dementia (Doraiswamy et al., “Amyloid-O Assessed by Florbetapir F18 PET and 18-month Cognitive Decline: A Multicenter Study,” Neurology 79:1636-44 (2012)). Interventions aiming at removal of Aβ deposits (including amyloid plaques) are hypothesized to slow the clinical progression of AD.

A second neuropathological hallmark of AD is the presence of intracellular neurofibrillary tangles containing hyperphosphorylated tau protein. Current disease models suggest that Aβ triggers tau pathology, with a more complex and synergistic interaction between Aβ and tau manifesting at later stages and driving disease progression (Busche et al., “Synergy Between Amyloid-O and Tau in Alzheimer's disease,” Nature Neuroscience 23:1183-93 (2020)).

Antibodies to Aβ and the use thereof in methods of treating diseases, such as, Alzheimer's disease, are known in the art. (See, e.g., U.S. Pat. Nos. 10,851,156; 10,738,109; 10,662,239; 10,654,917; 10,647,759; 10,603,367; 10,519,223; 10,494,425; 10,464,976; 10,112,991; 10,112,987; 10,035,847; 9,944,696; 9,939,452; 9,895,429; 9,834,598; 9,738,712; 9,585,956; 9,573,994; 9,382,312; 9,329,189; 9,309,309; 9,309,307; 9,272,031; 9,181,332; 9,176,150; 9,175,094; 9,146,244; 9,133,267; 9,125,846; 9,062,102; 9,051,364; 9,051,363; 9,034,334; 8,999,936; 8,916,165; 8,906,370; 8,906,367; 8,889,138; 8,796,439; 8,795,664; 8,710,193; 8,636,981; 8,614,299; 8,591,894; 8,507,206; 8,491,903; 8,470,321; 8,425,905; 8,420,093; 8,414,893; 8,409,575; 8,404,459; 8,398,978; 8,383,113; 8,337,848; 8,333,967; 8,323,654; 8,303,954; 8,268,973; 8,268,593; 8,246,954; 8,227,576; 8,222,002; 8,221,750; 8,173,127; 8,128,930; 8,128,928; 8,124,353; 8,124,076; 8,106,164; 8,105,594; 8,105,593; 8,025,878; 7,955,812; 7,939,075; 7,932,048; 7,927,594; 7,906,625; 7,902,328; 7,893,214; 7,892,545; 7,892,544; 7,871,615; 7,811,563; 7,807,165; 7,807,157; 7,790,856; 7,780,963; 7,772,375; 7,763,250; 7,763,249; 7,741,448; 7,731,962; 7,700,751; 7,625,560; 7,582,733; 7,575,880; 7,339,035; 7,320,790; 7,318,923; 7,256,273; 7,195,761; 7,189,819; 7,179,892; 7,122,374; 7,060,270; 6,815,175; 6,787,637; and 6,750,324; which are incorporated by reference in their entireties).

In one example, U.S. Pat. No. 8,679,498 (which is hereby incorporated by reference in its entirety, including the anti-N3pGlu Aβ antibodies disclosed therein) discloses anti-N3pGlu Aβ antibodies and methods of treating diseases, such as, Alzheimer's disease, with the antibodies. Passive immunization by long term chronic administration of antibodies against Aβ, including N3pGlu Aβ, found in deposits has been shown to disrupt the Aβ aggregates and promote the clearance of plaques in the brain in various animal models. Donanemab (disclosed in U.S. Pat. No. 8,679,498, referred to as antibody B12L) is an antibody directed at the pyroglutamate modification of the third amino acid of amyloid beta (N3pGlu Aβ) epitope that is present only in brain amyloid plaques. The mechanism of action of donanemab is the targeting and removal of existing amyloid plaque, which is a key pathological hallmark of AD.

The treatment and prevention strategy for anti-Aβ antibodies includes targeting an Aβ population of early symptomatic AD patients with existing brain amyloid load. This rationale is based on the amyloid hypothesis of AD, which states that the production and deposition of Aβ is an early and necessary event in the pathogenesis of AD. See, e.g., Selkoe, “The Origins of Alzheimer Disease: A is for Amyloid,” JAMA 283:1615-1617 (2000). Clinical support for this hypothesis comes from the demonstration that parenchymal Aβ levels are elevated before the appearance of symptoms of AD and supported by genetic variants of AD that overproduce brain Aβ and genetic variants that protect against Aβ production. See, e.g., Jonsson et al., “A Mutation in APP Protects Against Alzheimer's Disease and Age-related Cognitive Decline,” Nature 488 (7409):96-99 (2012) and Fleisher et al., “Associations Between Biomarkers and Age in the Presenilin 1 E280A Autosomal Dominant Alzheimer Disease Kindred: A Cross-sectional Study,” JAMA Neurol. 72:316-24 (2015).

Antibodies targeting amyloid plaques, such as antibodies targeting Aβ, have shown promise as a therapeutic for Alzheimer's disease in both preclinical and clinical studies. Despite this promise, antibodies targeting amyloid have failed to meet therapeutic endpoints in multiple clinical trials. The history of anti-amyloid clinical trials spans almost two decades and has, for the most part, cast doubt on the potential of such therapies to effectively treat AD (Aisen et al., “The Future of Anti-amyloid Trials,” The Journal of Prevention of Alzheimer's Disease 7 146-151 (2020)). To date, only a handful of AD treatments have been approved.

One of the challenges in treating Alzheimer's disease is that it is still principally diagnosed and treated based on symptoms like a psychiatric illness rather than based on brain pathology. Another challenge is a replication crisis faced during clinical trials where it is often difficult to obtain replicable results even if clinical trials are designed nearly identically. This is caused by two main factors. First, most trials set enrollment criteria based on symptoms rather than pathology. Thus, they end up enrolling a heterogenous population with wide variation in levels of underlying pathology or worse, patients with different underlying diseases. Accordingly, these patients progress at very different rates, and intra-group variability, measured by standard deviation of the mean for example, is quite large in AD trials. Second, the population heterogeneity problem is compounded by intra-subject noise in the outcome measurements.

Determining whether a subject having Aβ deposits is going to respond to anti-Aβ antibody treatment is uniquely challenging. This is partly because of the physiological and clinical heterogeneity amongst the subjects suffering from Aβ deposits. For example, determining if a patient with subtle cognitive symptoms, such as memory decline, suffers from prodromal or preclinical Alzheimer's disease (AD) and may progress to AD dementia within the near future remains a challenge for clinicians.

AD clinical trial placebo populations vary widely in trajectories of cognitive and functional decline (Veitch et al., “Understanding Disease Progression and Improving Alzheimer's Disease Clinical Trials: Recent Highlights from the Alzheimer's Disease Neuroimaging Initiative,” Alzheimer's & Dementia 15.1: 106-152 (2019)), which is believed to be due to heterogeneity in trial populations (Devi et al., “Heterogeneity of Alzheimer's Disease: Consequence for Drug Trials?” Alzheimer's Research & Therapy 10.1: 1-3 (2018)). Identifying and treating subjects who may benefit from a particular treatment continues to pose a substantial challenge. The task of properly identifying whether a patient may respond to anti-Aβ antibody treatments is of utmost importance for, e.g., a timely referral to a memory clinic, a correct and early AD diagnosis, initiation of symptomatic treatment, future planning, and initiating disease-modifying treatments.

Historically, trial cohorts have been selected by clinical features such as cognitive test score ranges and self-reported problems with memory. After years of failures, experts in the field have advocated testing anti-amyloid disease modifying therapies (DMTs) earlier in the course of the disease (Aisen, P. S., et al., “The future of anti-amyloid trials,” The Journal of Prevention of Alzheimer's Disease 7.3 (2020): 146-151.). However, several clinical studies for anti-amyloid DMTs have failed to meet their endpoints despite targeting patients in early stages of Alzheimer's disease. For instance, phase III clinical trial (Cread trial) for Crenezumab recruited patients with prodromal-to-mild AD. The results for this study were exclusively negative. No difference was found for both endpoints—primary and secondary—between the treatment versus placebo groups or within the prodromal versus mild AD subgroups (NCT03114657 at clinicaltrials.gov; Therapeutics: Crenezumab. Alzforum. AC Immune SA, Genentech, Hoffmann-La Roche; 2019 [cited 2020 Sep. 7]. Available from: alzforum.org/therapeutics/crenezumab). Similarly, a phase 11/III clinical trial (SCarlet RoAD trial) assessing the efficacy and safety of Gantenerumab in prodromal AD patients was terminated because the probability of obtaining efficacy on primary and secondary endpoints in the trial was low (Ostrowitzki et al., “A Phase III Randomized Trial of Gantenerumab in Prodromal Alzheimer's Disease,” Alzheimer's research & therapy 9.1: 1-15 (2017)).

Thus, a need exists for improved methods that properly identify whether a subject is going to respond to amyloid targeting therapeutics.

One aspect of the present invention is based on the discovery that Alzheimer's patients with low or moderate tau are responsive to treatment with anti-Aβ antibodies, and patients having high tau levels, even if clinically classified as preclinical or early-stage AD, may not be effectively treated with anti-Aβ antibodies. Identifying the subjects that are most responsive to treatment with an anti-Aβ antibody solves the 20+ year old problem of finding a clinically effective anti-amyloid treatment and as such reflects a significant advance in the art. Some aspects of the present invention are directed to diagnosing and treating patients based on their brain pathology. Selecting patients based on their brain pathology not only provides a more homogenous population in clinical trials and decreases noise to ensure highly replicable results but it also ensures proper identification of the stage of AD and its progression. Proper identification of the stage of AD also allows, e.g., for a timely referral to a memory clinic, a correct and early AD diagnosis, initiation of symptomatic treatment, future planning, and initiating disease-modifying treatments.

Some aspects of the present invention are related to identifying the stage/progression of AD in a patient based on i) the global or overall tau burden in the brain of a human subject, ii) the spread of tau in the subject's brain or portions thereof, and/or iii) based on the presence of one or two alleles of epsilon-4 of apolipoprotein E in the subject's genome (referred to herein as APOE e4 or APOE4).

In some embodiments, the patients can be stratified/identified/selected/treated based on the amount of tau present in the subject's brain (e.g., in the whole brain or in portions of the brain) and/or the presence of one or two alleles of APOE e4 in the subject's genome.

In other embodiments, the patients are stratified/identified/selected/treated based on stages of AD progression (e.g., based on the spread of tau in the brain) and/or the presence of one or two alleles of APOE e4 in the subject's genome. For example, during some stages, tau burden in an AD patient is isolated to frontal lobe or regions of the temporal lobe that do not include the posterolateral temporal region (PLT). Another stage of AD is where tau burden in an AD patient is limited to the posterolateral temporal (PLT) or occipital regions. Yet another stage of AD is when the tau burden in an AD patient is present in the parietal or precuneus region or in the frontal region along with tau burden in PLT or occipital regions. In some embodiments, the AD patient has one or two alleles of APOE e4 in the subject's genome and tau burden isolated to frontal lobe or regions of the temporal lobe that do not include the posterolateral temporal region (PLT). Another stage of AD is where the AD patient has one or two alleles of APOE e4 and tau burden is limited to the posterolateral temporal (PLT) or occipital regions. Yet another stage of AD is where the AD patient has one or two alleles of APOE e4 and tau burden is present in the parietal or precuneus region or in the frontal region along with tau burden in PLT or occipital regions.

The stratification of patients based on amount of tau in the brain or AD progression in portions of brain can be used to determine, e.g., whether a patient will respond to anti-Aβ antibody treatments. Stratification/selection of patient population based on amount of tau in the brain or AD progression in portions of brain is also helpful in solving the patient heterogeneity and replicability problems faced during design and performance of clinical trials. Identification of patients based on the amount of tau or AD progression is also helpful for, e.g., a timely referral to a memory clinic, a correct and early AD diagnosis, initiation of symptomatic treatment, future planning, and initiating disease-modifying treatments.

Additionally, Doody et al., “Phase 3 Trials of Solanezumab for Mild-to-Moderate Alzheimer's Disease,” NEJM, 370; 4, 311-321 (2014) state that “[n]o clear differential treatment effects on efficacy measures were observed between APOE ε4 carriers and noncarriers.” It has now been found that administering an anti-N3pGlu Aβ antibody to a human subject that has one or two alleles of APOE e4 (e.g., a carrier of APOE e4) provides unexpected and surprising efficacy when compared to non-carriers of one or more of those alleles. Thus, some embodiments involve administering doses of anti-N3pGlu Aβ antibodies to patients who have that allele as a means of slowing the cognitive decline of those patients. Specifically, it has been found that there is a greater effect in carriers of APOE e4 than in non-carriers when the patients are administered anti-N3pGlu Aβ antibodies. This means that the patients that have APOE e4 have less cognitive decline than non-carriers, when measured using various clinical measurements and at various endpoints. Therefore, patients can be stratified/identified/selected/treated based on levels of tau, stages of AD progression (e.g., based on the spread of tau in the brain) and/or the presence of one or two alleles of APOE e4 in the subject's genome.

One aspect of the present invention provides for human subjects that are responsive to treatment or prevention of a disease characterized by amyloid beta (Aβ) deposits in the brain of a human subject. In some embodiments, of this aspect of the present invention, the responsive human subjects include human subjects having low to moderate tau burden or very low to moderate tau burden. In some embodiments, of this aspect of the present invention, the responsive human subjects include human subjects having low to moderate tau burden or very low to moderate tau burden and/or one or two alleles of APOE e4. In some embodiments of this aspect of the present invention, the responsive human subjects exclude human subjects with high tau burden and a change in the integrated Alzheimer's Disease Rating Scale (iADRS) of about −20 or more after about the past 18 months. In some embodiments, the anti-Aβ antibody of the present invention is administered to the responsive human subjects for treatment or prevention of a disease characterized by amyloid beta (Aβ) deposits in the brain of a human subject.

One aspect of the present invention is related to a method of treating or preventing a disease characterized by amyloid beta (Aβ) deposits in the brain of a human subject who has been determined to have very low to moderate tau burden or low to moderate tau burden, the method comprising administering to the subject one or more doses of an anti-Aβ antibody. In some embodiments, the methods comprise: i) administering to the human subject one or more first doses of an anti-Aβ antibody (e.g., one or more first doses of about 100 mg to about 700 mg of an anti-Aβ antibody), wherein each first dose is administered once about every 4 weeks and ii) about four weeks after administering the one or more first doses, administering to the human subject one or more second doses of an anti-antibody (e.g., one or more second doses of greater than 700 mg to about 1400 mg of the anti-Aβ antibody), wherein each second dose is administered once about every 4 weeks. In some embodiments, the Alzheimer's patient has one or two alleles of APOE e4.

Another aspect of the present invention is related to a method of treating or preventing a disease characterized by amyloid beta (Aβ) deposits in the brain of a human subject comprising: determining whether the human subject has very low to moderate tau burden or low to moderate tau burden; and if the human subject has very low to moderate tau burden or low to moderate tau burden, then administering to the human subject one or more doses of an anti-Aβ antibody. In some embodiments, the methods comprise i) administering to the human subject one or more first doses of about 100 mg to about 700 mg of an anti-Aβ antibody, wherein each first dose is administered once about every 4 weeks and ii) about four weeks after administering the one or more first doses, administering to the human subject one or more second doses of greater than 700 mg to about 1400 mg of an anti-Aβ antibody, wherein each second dose is administered once about every 4 weeks.

Another aspect of the present invention is related to a method of treating or preventing a disease characterized by amyloid beta (Aβ) deposits in the brain of a human subject comprising: determining whether the human subject has one or two alleles of APOE e4, very low to moderate tau burden, and/or low to moderate tau burden; and if the human subject has one or two alleles of APOE e4, very low to moderate tau burden, and/or low to moderate tau burden, then administering to the human subject one or more doses of an anti-Aβ antibody. In some embodiments, the methods comprise i) administering to the human subject one or more first doses of about 100 mg to about 700 mg of an anti-Aβ antibody, wherein each first dose is administered once about every 4 weeks and ii) about four weeks after administering the one or more first doses, administering to the human subject one or more second doses of greater than 700 mg to about 1400 mg of an anti-Aβ antibody, wherein each second dose is administered once about every 4 weeks.

Another aspect of the invention is related to a method of treating or preventing a disease characterized by amyloid beta (Aβ) deposits in the brain of a human subject who has been determined as not having high tau burden, the method comprising administering to the human subject an anti-Aβ antibody. In some embodiments, the method comprises: i) administering to the human subject one or more first doses of about 100 mg to about 700 mg of an anti-Aβ antibody, wherein each first dose is administered once about every 4 weeks and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined as not having high tau burden and has one or two alleles of APOE e4.

Another aspect of the invention is related to a method of treating or preventing a disease characterized by amyloid beta (Aβ) deposits in the brain of a human subject comprising: determining whether the human subject has high tau burden; and if the human subject does not have high tau burden, then administering to the human subject on or more doses of an anti-Aβ antibody. In some embodiments, the methods comprise: i) administering to the human subject one or more first doses of about 100 mg to about 700 mg of an anti-Aβ antibody, wherein each first dose is administered once about every 4 weeks and ii) about four weeks after administering the one or more first doses, administering to the human subject one or more second doses of greater than 700 mg to about 1400 mg of the anti-Aβ antibody, wherein each second dose is administered once about every 4 weeks.

Another aspect of the invention is related to a method of treating or preventing a disease characterized by amyloid beta (Aβ) deposits in the brain of a human subject comprising: determining whether the human subject has high tau burden and has one or two alleles of APOE e4; and if the human subject does not have high tau burden and has one or two alleles of APOE e4, then administering to the human subject on or more doses of an anti-Aβ antibody. In some embodiments, the methods comprise: i) administering to the human subject one or more first doses of about 100 mg to about 700 mg of an anti-Aβ antibody, wherein each first dose is administered once about every 4 weeks and ii) about four weeks after administering the one or more first doses, administering to the human subject one or more second doses of greater than 700 mg to about 1400 mg of the anti-Aβ antibody, wherein each second dose is administered once about every 4 weeks.

Another aspect of the present invention is related to a method of treating or preventing a disease characterized by amyloid beta (Aβ) deposits in the brain of a human subject comprising administering to the human subject an effective amount of an anti-Aβ antibody, wherein the human subject has been determined as having a very low to moderate tau burden or low to moderate tau burden.

Another aspect of the present invention is related to a method of treating or preventing a disease characterized by amyloid beta (Aβ) deposits in the brain of a human subject comprising administering to the human subject an effective amount of an anti-Aβ antibody, wherein the human subject has been determined as having one or two alleles of APOE e4 and a very low to moderate tau burden or low to moderate tau burden.

Another aspect of the present invention is related to a method of treating or preventing a disease characterized by amyloid beta (Aβ) deposits in the brain of a human subject comprising determining whether the human subject has low to moderate tau burden or a very low to moderate tau burden; and if the human subject has low to moderate tau burden or a very low to moderate tau burden, then: administering to the human subject an effective amount of an anti-Aβ antibody.

Another aspect of the present invention is related to a method of treating or preventing a disease characterized by amyloid beta (Aβ) deposits in the brain of a human subject comprising determining whether the human subject has one or two alleles of APOE e4 and low to moderate tau burden or a very low to moderate tau burden; and if the human subject has one or two alleles of APOE e4 and low to moderate tau burden or a very low to moderate tau burden, then: administering to the human subject an effective amount of an anti-Aβ antibody.

Another aspect of the present invention is related to a method of treating or preventing a disease characterized by amyloid beta (Aβ) deposits in the brain of a human subject comprising administering to the human subject an effective amount of an anti-Aβ antibody, wherein the human subject has been determined as not having a high tau burden and the human subject has not demonstrated a decrease in the integrated Alzheimer's Disease Rating Scale (iADRS) of greater than about −20 over about the past 18 months. In some embodiments, the human subject has one or two alleles of APOE e4.

Another aspect of the present invention is related to a method of treating or preventing a disease characterized by amyloid beta (Aβ) deposits in the brain of a human subject comprising determining whether the human subject has high tau burden; and if the human subject does not have high tau burden, then: administering to the human subject an effective amount of an anti-Aβ antibody.

Another aspect of the present invention is related to a method of treating or preventing a disease characterized by amyloid beta (Aβ) deposits in the brain of a human subject comprising determining whether the human subject has high tau burden and one or two alleles of APOE e4; and if the human subject has one or two alleles of APOE e4 and does not have high tau burden, then: administering to the human subject an effective amount of an anti-Aβ antibody.

In some aspects of the disclosed methods, an anti-Aβ antibody may be used to decrease, prevent further increase, or slow the rate of tau burden/accumulation in different portions of a human brain, e.g., in different lobes of the human brain of a human subject. In some embodiments, the anti-Aβ antibodies are used to decrease, prevent further increase, or slow the rate of tau burden/accumulation in the frontal lobe of the human brain. In some embodiments, the anti-Aβ antibodies are used to decrease, prevent further increase, or slow the rate of tau burden/accumulation in the parietal lobe of the human brain. In some embodiments, the anti-Aβ antibodies are used to decrease, prevent further increase, or slow the rate of tau burden/accumulation in the occipital lobe of the human brain. In some embodiments, the anti-Aβ antibodies are used to decrease, prevent further increase, or slow the rate of tau burden/accumulation in the temporal lobe of the human brain. In some embodiments, the anti-Aβ antibodies are used to decrease, prevent further increase, or slow the rate of tau burden/accumulation in the posterolateral temporal lobe. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks.

An aspect of the present invention is related to a method of treating or preventing a disease characterized by amyloid beta (Aβ) deposits in the brain of a human subject who has been determined to have tau burden in the temporal lobe of the brain wherein the method comprises administering an anti-Aβ antibody to the human subject. Another aspect of the invention is related to a method of treating or preventing a disease characterized by amyloid beta deposits in the brain of a human subject comprising determining whether the human subject has a tau burden in the temporal lobe of the brain and administering an anti-Aβ antibody to the human subject. In some embodiments, the human subject has a tau burden in the posterolateral temporal lobe. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta (Aβ) deposits in the brain of a human subject who has been determined to have tau burden in the occipital lobe of the brain wherein the method comprises administering an anti-Aβ antibody to the human subject. Another aspect of the invention is related to a method of treating or preventing a disease characterized by amyloid beta (Aβ) deposits in the brain of a human subject comprising determining whether the human subject has a tau burden in the occipital lobe of the brain and administering an anti-Aβ antibody to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta (Aβ) deposits in the brain of a human subject who has been determined to have tau burden in the parietal lobe of the brain wherein the method comprises administering an anti-Aβ antibody to the human subject. Another aspect of the invention is related to a method of treating or preventing a disease characterized by amyloid beta (Aβ) deposits in the brain of a human subject comprising determining whether the human subject has a tau burden in the parietal lobe of the brain and administering an anti-Aβ antibody to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta deposits in the brain of a human subject who has been determined to have tau burden in the frontal lobe of the brain wherein the method comprises administering an anti-Aβ antibody to the human subject. Another aspect of the invention is related to a method of treating or preventing a disease characterized by amyloid beta deposits in the brain of a human subject comprising determining whether the human subject has a tau burden in the frontal lobe of the brain and administering an anti-Aβ to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta deposits in the brain of a human subject who has been determined to have tau burden in the posterolateral temporal (PLT) and/or occipital lobe of the brain wherein the method comprises administering an anti-Aβ antibody to the human subject. Another aspect of the invention is related to a method of treating or preventing a disease characterized by amyloid beta deposits in the brain of a human subject comprising determining whether the human subject has a tau burden in the posterolateral temporal (PLT) and/or occipital lobe of the brain and administering an anti-Aβ antibody to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta deposits in the brain of a human subject who has been determined to have tau burden in i) parietal or precuneus region or ii) in frontal region along with tau burden in PLT or occipital regions of the brain wherein the method comprises administering an anti-Aβ antibody to the human subject. Another aspect of the invention is related to a method of treating or preventing a disease characterized by amyloid beta deposits comprising determining whether the human subject has a tau burden in i) parietal or precuneus region or ii) in the frontal region along with tau burden in PLT or occipital regions of the brain and administering an anti-Aβ antibody to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

Another aspect of the present invention is related to a method of treating or preventing a disease characterized by amyloid beta deposits in the brain of a human subject who has been determined to have tau burden i) isolated to frontal lobe or ii) in regions of the temporal lobe that do not include the posterolateral temporal region (PLT) of the brain wherein the method comprises administering an anti-Aβ antibody to the human subject. Another aspect of the invention is related to a method of treating or preventing a disease characterized by amyloid beta deposits comprising determining whether the human subject has a tau burden i) isolated to frontal lobe or ii) in regions of the temporal lobe that do not include the posterolateral temporal region (PLT) of the brain and administering an anti-Aβ antibody to the human subject. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

In some aspects, the present invention is related to a method of selecting a human subject for treatment or prevention of a disease characterized by amyloid beta deposits in the brain of a human subject. In some embodiments, the human subject is selected based on the amount of global (overall) tau in the brain of the human subject. For example, the human subject is selected for treatment or prevention of a disease characterized by amyloid beta deposits in the brain because the patient has very low to moderate tau in the brain. In another embodiment, the human subject is selected for treatment or prevention of a disease characterized by amyloid beta deposits in the brain because the patient has low to moderate tau (or intermediate tau) in the brain. In another embodiment, the human subject is excluded from treatment or prevention of a disease characterized by amyloid beta deposits in the brain because the patient has high tau in the brain. In some embodiments, the human subject is selected based on progression of AD in the brain of the human subject. For example, the human subject is selected for treatment or prevention of a disease characterized by amyloid beta deposits in the brain because the patient has a tau burden present in the frontal lobe of the brain. In another embodiment, the human subject is selected for treatment or prevention of a disease characterized by amyloid beta deposits in the brain because the patient has a tau burden present in the parietal lobe of the brain. In another embodiment, the human subject is selected for treatment or prevention of a disease characterized by amyloid beta deposits in the brain because the patient has a tau burden present in the occipital lobe of the brain. In another embodiment, the human subject is selected for treatment or prevention of a disease characterized by amyloid beta deposits in the brain because the patient has tau burden present in the temporal lobe of the brain. In some embodiments, the human subject is selected for treatment or prevention of a disease characterized by amyloid beta deposits in the brain because the patient has tau burden present in the posterolateral temporal (PLT) and/or occipital lobe of the brain. In some embodiments, the human subject is selected for treatment or prevention of a disease characterized by amyloid beta deposits in the brain because the patient has tau burden present in i) parietal or precuneus region or ii) in frontal region along with tau burden in PLT or occipital regions of the brain. In some embodiments, the human subject is selected for treatment or prevention of a disease characterized by amyloid beta deposits in the brain because the patient has tau burden i) isolated to frontal lobe or ii) in regions of the temporal lobe that do not include the posterolateral temporal region (PLT) of the brain. In some embodiments, the human subject is administered i) one or more first doses of about 100 mg to about 700 mg of the anti-Aβ antibody, wherein each first dose is administered once about every four weeks; and ii) about four weeks after administering the one or more first doses, administering to the human subject one or more second doses of greater than 700 mg to about 1400 mg of the anti-Aβ antibody, wherein each second dose is administered once about every 4 weeks. In some embodiments, the human subject has been determined to have or has one or two alleles of APOE e4.

In some embodiments, the subject described in the various aspects of the present invention has been determined to have a posterior-lateral temporal lobe tau burden. In some embodiments, the subject described in the various aspects of the present invention has been determined to have posterior-lateral temporal lobe and occipital lobe tau burden. In some embodiments, the subject described in the various aspects of the present invention has been determined to have posterior-lateral temporal lobe tau burden, occipital lobe tau burden, and/or parietal lobe tau burden. In some embodiments, the subject described in the various aspects of the present invention has been determined to have posterior-lateral temporal lobe tau burden, occipital lobe tau burden, parietal lobe tau burden, and/or frontal lobe tau burden. In some embodiments, the subject described in the various aspects of the present invention has been determined to have posterior-lateral temporal lobe tau burden, occipital lobe tau burden, parietal lobe tau burden, and/or frontal lobe tau burden. In some embodiments, the subject described in the various aspects of the present invention has been determined to have posterior-lateral temporal lobe tau burden, occipital lobe tau burden, parietal lobe tau burden, and/or frontal lobe tau burden which corresponds to a tau burden of greater than 1.46 SUVr based on PET imaging. In some embodiments, the human subject has been determined to have or has one or two alleles of APOE e4.

In some embodiments, tau burden in a portion of the human brain (e.g., in a lobe of the brain) can be used to determine whether administration of the anti-Aβ antibody should be discontinued. For instance, a decrease, a prevention of further increase, or a slowing in the rate of tau burden/accumulation in a portion of the brain can be used as metric to determine the duration of administration of the anti-Aβ antibody. In some embodiments, anti-Aβ antibody is administered to the subject until there is a decrease, a prevention of further increase, or a slowing in the rate of tau burden/accumulation in the temporal lobe, the occipital lobe, the parietal lobe, or the frontal lobe.

In some embodiments, the tau burden present in a portion of the brain of a human subject (e.g., in a defined lobe of the brain of a human subject) can be used for selection of optimal treatment regimens or for administration of therapeutic modalities in combination with an anti-Aβ antibody. For example, the presence of tau burden in the frontal lobe of the brain of an amyloid positive human subject can be used as a metric to determine whether the human subject will benefit from administration of an anti-Aβ antibody alone or in combination with an anti-tau antibody. In some embodiments, an anti-Aβ antibody in combination with an anti-tau antibody may be administered to a subject in order to decrease, to prevent further increase, or to slow the rate of tau burden/accumulation in different portions of a human brain, e.g., in different lobes of the human brain of a human subject. In some embodiments, the tau burden in different portions of a human brain, e.g., in different lobes of the human brain of a human subject, can be used for i) tracking a patient's response to treatment, or ii) determining when a therapy may need to be reinitiated.

In some embodiments, the antibodies, methods, or dosing regimens described in various aspects of the present invention cause: i) reduction in Aβ deposits in the brain of the human subject and/or ii) slows cognitive decline or functional decline in the human subject. In some embodiments, the antibodies, methods, or dosing regimens described herein this method results in reduction of amyloid plaques.

In some embodiments, the anti-Aβ antibodies described in various aspects of the present invention i) include, ii) may be replaced with, or iii) used along with anti-N3pGlu Aβ antibodies such as:
an anti-N3pGlu Aβ antibody comprising: light chain complementarity determining region 1 (LCDR1) having an amino acid sequence of SEQ ID NO: 5, light chain complementarity determining region 2 (LCDR2) having an amino acid sequence of SEQ ID NO: 6, and light chain complementarity determining region 3 (LCDR3) having an amino acid sequence of SEQ ID NO: 7 or an amino acid sequence having at least 95% homology to light chain complementarity determining region 1 (LCDR1) of SEQ ID NO: 5, an amino acid sequence having at least 95% homology to light chain complementarity determining region 2 (LCDR2) of SEQ ID NO: 6, and an amino acid sequence having at least 95% homology to light chain complementarity determining region 3 (LCDR3) of SEQ ID NO: 7;
an anti-N3pGlu Aβ antibody comprising: heavy chain complementarity determining region 1 (HCDR1) having an amino acid sequence of SEQ ID NO: 8, heavy chain complementarity determining region 2 (HCDR2) having an amino acid sequence of SEQ ID NO: 9, and heavy chain complementarity determining region 3 (HCDR3) having an amino acid sequence of SEQ ID NO: 10 or an amino acid sequence having at least 95% homology to heavy chain complementarity determining region 1 (HCDR1) of SEQ ID NO: 8, an amino acid sequence having at least 95% homology to heavy chain complementarity determining region 2 (HCDR2) of SEQ ID NO: 9, and an amino acid sequence having at least 95% homology to heavy chain complementarity determining region 3 (HCDR3) of SEQ ID NO: 10;
an anti-N3pGlu Aβ antibody comprising: light chain complementarity determining region 1 (LCDR1) having an amino acid sequence of SEQ ID NO: 5, light chain complementarity determining region 2 (LCDR2) having an amino acid sequence of SEQ ID NO: 6, light chain complementarity determining region 3 (LCDR3) having an amino acid sequence of SEQ ID NO: 7, heavy chain complementarity determining region 1 (HCDR1) having an amino acid sequence of SEQ ID NO: 8, heavy chain complementarity determining region 2 (HCDR2) having an amino acid sequence of SEQ ID NO: 9, and heavy chain complementarity determining region 3 (HCDR3) having an amino acid sequence of SEQ ID NO: 10 or amino acid sequence having at least 95% homology to light chain complementarity determining region 1 (LCDR1) of SEQ ID NO: 5, amino acid sequence having at least 95% homology to light chain complementarity determining region 2 (LCDR2) of SEQ ID NO: 6, amino acid sequence having at least 95% homology to light chain complementarity determining region 3 (LCDR3) of SEQ ID NO: 7, amino acid sequence having at least 95% homology to heavy chain complementarity determining region 1 (HCDR1) of SEQ ID NO: 8, amino acid sequence having at least 95% homology to heavy chain complementarity determining region 2 (HCDR2) of SEQ ID NO: 9, and amino acid sequence having at least 95% homology to heavy chain complementarity determining region 3 (HCDR3) of SEQ ID NO: 10;
an anti-N3pGlu Aβ antibody comprising: a LCVR and a HCVR, wherein said LCVR comprises: LCDR1, LCDR2 and LCDR3 and HCVR comprises HCDR1, HCDR2 and HCDR3, which are selected from the group consisting of LCDR1 is SEQ ID NO: 5, LCDR2 is SEQ ID NO: 6, LCDR3 is SEQ ID NO: 7, HCDR1 is SEQ ID NO: 8, HCDR2 is SEQ ID NO: 9, and HCDR3 is SEQ ID NO: 10 or a LCVR and a HCVR, wherein said LCVR comprises LCDR1, LCDR2 and LCDR3 and HCVR comprises HCDR1, HCDR2 and HCDR3, which are selected from the group consisting of LCDR1 having at least 95% homology to SEQ ID NO: 5, LCDR2 having at least 95% homology to SEQ ID NO: 6, LCDR3 having at least 95% homology to SEQ ID NO: 7, HCDR1 having at least 95% homology to SEQ ID NO: 8, HCDR2 having at least 95% homology to SEQ ID NO: 9, and HCDR3 having at least 95% homology to SEQ ID NO: 10.
an N3pGlu Aβ antibody comprising a light chain (LC) comprising: the amino acid sequence of SEQ ID NO: 3 or amino acid sequence having at least 95% homology to SEQ ID NO: 3;
an N3pGlu Aβ antibody comprising a heavy chain (HC) comprising: the amino acid sequence of SEQ ID NO: 4 or amino acid sequence having at least 95% homology to SEQ ID NO: 4;
an anti-N3pGlu Aβ antibody comprising a LC and a HC, wherein the LC comprises the amino acid sequence of SEQ ID NO: 3 and the HC comprises the amino acid sequence of SEQ ID NO: 4 or wherein the LC comprises amino acid sequence having at least 95% homology to SEQ ID NO: 3 and the HC comprises amino acid sequence having at least 95% homology to SEQ ID NO: 4;
an anti-N3pGlu Aβ antibody comprising two light chains and two heavy chains, wherein the LC comprises amino acid sequence of SEQ ID NO: 3 or amino acid sequence having at least 95% homology to SEQ ID NO: 3 and the HC comprises the amino acid sequence of SEQ ID NO: 4 or amino acid sequence having at least 95% homology to SEQ ID NO: 4.
an N3pGlu Aβ antibody comprising a LCVR comprising the amino acid sequence of SEQ ID NO: 1 or amino acid sequence having at least 95% homology to SEQ ID NO: 1;
an N3pGlu Aβ antibody comprising a HCVR comprising the amino acid sequence of SEQ ID NO: 2 or amino acid sequence having at least 95% homology to SEQ ID NO: 2.
an N3pGlu Aβ antibody comprising a LCVR and a HCVR wherein the LCVR comprises the amino acid sequence of SEQ ID NO: 1 or amino acid sequence having at least 95% homology to SEQ ID NO: 1; and the HCVR comprises the amino acid sequence of SEQ ID NO: 2 or amino acid sequence having at least 95% homology to SEQ ID NO: 2.

The anti-Aβ antibodies described in various aspects of the present invention i) include, ii) may be replaced with, or iii) used along with anti-Aβ antibodies disclosed in the art such as the antibodies donanemab, aducanumab, bapineuzumab, GSK933776, solanezumab, crenezumab, ponezumab, lecanemab (BAN2401), and gantenerumab. In some embodiments, the anti-Aβ antibodies of the present invention include kappa LC and IgG HC. In a particular embodiment, the anti-Aβ antibodies of the present invention are of the human IgG1 isotype.

In some embodiments of the disclosed methods, the human subject is administered one or more first doses of about 100 mg to about 700 mg of the anti-Aβ antibody as described herein. In some embodiments, the one or more first doses are administered to the human subject such that each first dose is administered once every four weeks. In embodiments, the first dose is administered to the subject once. In some embodiments, the first dose is administered to the subject twice wherein each first dose is administered once every four weeks. In some embodiments, the first dose is administered to the subject three times wherein each first dose is administered once every four weeks.

In some embodiments, the subject is administered one first dose, two first doses, or three first doses of about 100 mg to about 700 mg, wherein each first dose is administered once about every four weeks. In a particular embodiment, the human subject is administered three first doses of about 700 mg wherein each first dose is administered once about every four weeks. In some embodiments, the human subject is administered the first dose once, two times, or three times before administering the second dose.

In some embodiments, three first doses of about 700 mg are administered to the subject once every 4 weeks for a duration of 12 weeks followed by second doses of about 1400 mg. In some embodiments, the one or more first doses of about 700 mg are administered to the subject once every 4 weeks over a duration of about 3 months followed by second doses of about 1400 mg.

In some embodiments, the first dose is about 100 mg, about 200 mg, about 300 mg, about 400 mg, about 500 mg, about 600 mg, about 700 mg. In some embodiments, the first dose from about 1 mg/kg to about 10 mg/kg of the anti-Aβ antibody. In particular embodiments, the subject is administered up to three first doses of about 1 mg/kg to about 10 mg/kg. In some embodiments, the subject is administered one first dose, two first doses, or three first doses of about 1 mg/kg to about 10 mg/kg. In one particular embodiment, the subject is administered three first doses of about 10 mg/kg once every four weeks. In some embodiments, the first dose is about 1 mg/kg, about 2 mg/kg, about 3 mg/kg, about 4 mg/kg, about 5 mg/kg, about 6 mg/kg, about 7 mg/kg, about 8 mg/kg, about 9 mg/kg or about 10 mg/kg.

In a particular embodiment, the first dose is administered once every 4 weeks or once every month. In one embodiment, the subject is administered three first doses of about 10 mg/kg once every 4 weeks. In some embodiments, the first dose of the anti-Aβ antibody is administered to the subject for about one month, about two months, or about three months.

In some embodiments, the subject is administered one or more second doses of greater than 700 mg to about 1400 mg of the anti-Aβ antibody. In some embodiments, the subject is administered one or more second doses of greater than 700 mg to about 1400 mg of the anti-Aβ antibody wherein each second dose is administered once about every 4 weeks. In some embodiments, the second dose is administered 4 weeks after the one or more first doses.

In embodiments, the subject is administered one or more second doses of greater than 700 mg. In some embodiments, the subject is administered one or more second doses of about 1400 mg. In some embodiments, the second dose is greater than 700 mg, about 800 mg, about 900 mg, about 1000 mg, about 1100 mg, about 1200 mg, about 1300 mg or about 1400. In a particular embodiment, the second dose is administered once every 4 weeks. In one embodiment, the subject is administered one or more second doses of greater than 700 mg once every 4 weeks. In one embodiment, the subject is administered one or more second doses of about 1400 mg once every 4 weeks.

An MRI scan may be administered to the human subject to check/evaluate any adverse event(s) caused by the administration of anti-A antibody. In some embodiments, the human subject is administered an MRI scan in between administration of doses of the anti-Aβ antibody. In some embodiments, the human subject is administered an MRI scan before increasing the dose of the anti-Aβ antibody, e.g., from 700 mg to 1400 mg. In some embodiments, the human subject is administered an MRI scan before administering a 1400 mg dose. In some embodiments, the human subject is administered an MRI scan before administering a 20 mg/kg dose. In some embodiments, the human subject is administered an MRI scan after the last dose 700 mg dose. In some embodiments, the human subject is administered an MRI scan after the last dose 10 mg/kg dose.

In some embodiments, the subject is administered one or more second doses of greater than 10 mg/kg to about 20 mg/kg of the anti-Aβ antibody. In some embodiments, the second dose is greater than 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg or about 20 mg/kg. In one embodiment, the subject is administered one or more second doses of greater than 10 mg/kg. In one embodiment, the subject is administered one or more second doses of about 20 mg/kg. In an embodiment, the first dose is administered once every month. In one embodiment, the subject is administered one or more second doses of greater than 10 mg/kg, wherein each second dose is administered once every 4 weeks or once every month. In one embodiment, the subject is administered one or more second doses of about 20 mg/kg, wherein each second dose is administered once every 4 weeks or once every month.

In some embodiments, the first dose of the anti-Aβ antibody is administered to the subject once followed by one or more second doses, wherein the second dose is administered 4 weeks after the one or more first doses and once every 4 weeks thereafter. In some embodiments, the first doses of the anti-Aβ antibody are administered to the subject two times (once every four weeks) followed by one or more second doses which are administered after 4 weeks of the first doses and once every 4 weeks thereafter. In some embodiments, the first doses of the anti-Aβ antibody are administered to the subject three times (once every four weeks) followed by one or more second doses which are administered after 4 weeks of the first doses and once every 4 weeks thereafter.

In some embodiments, the subject is treated with one or more first doses, one or more second doses of about 1400 mg, and subsequently with one or more second doses of greater than 700 mg to about 1300 mg. In one embodiment, the subject is treated with one or more first doses of about 700 mg, one or more second doses of about 1400 mg, and subsequently with one or more doses of about 700 mg.

In some embodiments, the dosing regimen of the present invention includes one or more additional doses (also referred to herein as third dose(s)) after the one or more first doses of about 100 mg to about 700 mg and the one or more second doses of greater than 700 mg to about 1400 mg. In some embodiments, the third dose is administered to the subject to reduce the deposition of Aβ in the brain of the subject, prevent further deposition of Aβ in the brain of the subject, prevent further cognitive decline, prevent memory loss, or prevent functional decline. The third dose could be from about 100 mg to about 1400 mg. In some embodiments different or same antibodies are used for the first dose, the second dose, and the third doses. In some embodiments, a different Aβ targeting antibody is administered in the third dose. For instance, some embodiments of the present invention include i) administering to the human subject one or more first doses of about 100 mg to about 700 mg of the anti-Aβ antibody, wherein each first dose is administered once about every 4 weeks; ii) about four weeks after administering the one or more first doses, administering to the human subject one or more second doses of greater than 700 mg to about 1400 mg of the anti-Aβ antibody, wherein each second dose is administered once about every 4 weeks, and iii) subsequently administering one or more third doses of about 100 mg to about 1400 mg of the anti-Aβ antibody. In some embodiments, one or more third doses of the anti-Aβ antibodies of the present invention can be administered to the subject every 2 or 4 weeks, every month, every 1 year, every 2 years, every 3 years, every 4 years, every 5 years, or every 10 years. In some embodiments, the third dose is given every 2 weeks. In some embodiments, the third dose is given every 4 weeks. In some embodiments, the third dose is given every year. In an embodiment, the third dose is given every 2 years. In another embodiment, the third dose is given every 3 years. In another embodiment, the third dose of the antibody is given every 5 years. In another embodiment, the third dose of the antibody is given every 10 years. In another embodiment, the third dose of the antibody is given every 2 to 5 years. In another embodiment, the third dose of the antibody is given every 5 to 10 years.

In some embodiments, the anti-Aβ antibody is administered to the subject for a duration sufficient to treat or prevent the disease. In some embodiments, the anti-Aβ antibody (including the first doses of the antibody and the second doses of the antibody) is administered to the subject for a duration of up to about 72 weeks, optionally, once every 4 weeks or once every month. In some embodiments, the anti-Aβ antibody (including the first doses of the antibody and the second doses of the antibody) is administered to the subject for a duration of up to about 98 weeks, optionally, once every 4 weeks or once every month. In some embodiments, the anti-Aβ antibody (including the first doses of the antibody and the second doses of the antibody) is administered to the subject for a duration of up to about 124 weeks, optionally, once every 4 weeks or once every month. In some embodiments, the anti-Aβ antibody (including the first doses of the antibody and the second doses of the antibody) is administered to the human subject until normal level of amyloid is achieved in the subject. In some embodiments, the anti-Aβ antibody (including the first doses of the antibody and the second doses of the antibody) is administered to the human subject until the subject becomes amyloid negative (a subject is considered amyloid negative when the amyloid plaque level in the brain of the subject is less than 24.1 CL). In some embodiments, the anti-Aβ antibody (including the first doses of the antibody and the second doses of the antibody) is administered to the human subject until the brain amyloid plaque level in the subject is in normal range or cleared. The normal range of amyloid plaque is defined as demonstrating an amyloid plaque level of 25 centiloids or lower for two consecutive PET scans at least 6 months apart or a single PET scan demonstrating a plaque level of less than 11 centiloids. In the present disclosure, the term “normal range” of amyloid plaque in brain is used interchangeably with brain amyloid plaque is “cleared.”

In some embodiments, the anti-Aβ antibody (including the first doses of the antibody and the second doses of the antibody) is administered to the subject for a duration of up to about 18 months, optionally, once every 4 weeks or once every month. In some embodiments, the anti-Aβ antibody (including the first doses of the antibody and the second doses of the antibody) is administered to the subject for a duration of up to about 24 months, optionally, once every 4 weeks or once every month. In some embodiments, the anti-Aβ antibody (including the first doses of the antibody and the second doses of the antibody) is administered to the subject for a duration of up to about 30 months, optionally, once every 4 weeks or once every month.

In one embodiment, the subject is administered three first doses of 700 mg once every four weeks and then second doses of 1400 mg once every four weeks for a duration of up to 72 weeks. In some embodiments, the anti-Aβ antibody (including, e.g., the first doses of the antibody and the second doses of the antibody) is administered to the subject for a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or about 76 weeks. In some embodiments, the anti-Aβ antibody (including, e.g., the first doses of the antibody and the second doses of the antibody) is administered to the subject for a duration of about 76 weeks, about 80 weeks, about 84 weeks, about 88 weeks, about 92 weeks, about 96 weeks, about 100 weeks, about 104 weeks, about 108 weeks, about 112 weeks, about 116 weeks, or about 120 weeks.

In a particular embodiment, the anti-Aβ antibody is administered to the subject for a duration of about 24 weeks. In a particular embodiment, the antibody is administered to the subject for a duration of about 28 weeks. In a particular embodiment, the antibody is administered to the subject for a duration of about 52 weeks. In a particular embodiment, the antibody is administered to the subject for a duration of about 72 weeks.

In some embodiments, the anti-Aβ antibody (including, e.g., the first doses of the antibody and the second doses of the antibody) is administered to the subject for a duration of from about 1 month to about 18 months. In some embodiments, the anti-Aβ antibody is administered to the subject for a duration of about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, or about 18 months. In some embodiments, the anti-Aβ antibody is administered to the subject for a duration of about 19 months, about 20 months, about 21 months, about 22 months, about 23 months, about 24 months, about 25 months, about 26 months, about 27 months, about 28 months, about 29 months, or about 30 months.

In some embodiments, the antibody is administered to the subject until brain amyloid plaque reaches a normal range or is cleared.

In a particular embodiment, the antibody is administered to the subject for a duration of about 3 months. In a particular embodiment, the antibody is administered to the subject for a duration of about 6 months. In a particular embodiment, the antibody is administered to the subject for a duration of about 12 months. In a particular embodiment, the antibody is administered to the subject for a duration of about 18 months.

In some embodiments, the human subject is administered the anti-Aβ antibody for a duration sufficient to treat or prevent the disease characterized by amyloid beta (Aβ) deposits in the brain of the human subject. In some embodiments, the human subject is administered the anti-Aβ antibody (including, e.g., the first dose and/or the second dose) for a duration sufficient to bring the amyloid plaque in the subject's brain to a normal range. The normal range of amyloid plaque is defined as demonstrating an amyloid plaque level of 25 centiloids or lower for two consecutive PET scans at least 6 months apart or a single PET scan demonstrating a plaque level of less than 11 centiloids.

In some embodiments, the antibody of the present invention is administered to the subject until amyloid plaque level in the subject is about 25 centiloids or lower. In some embodiments, the amyloid plaque is measured by PET imaging. In other embodiments, the antibody of the present invention is administered to the subject until the amyloid plaque level in the subject is about 25 centiloids or lower for two consecutive PET imaging scans. In some embodiments, the two consecutive PET imaging scans are at least 6 months apart. In some embodiments, the antibody of the present invention is administered to the subject until the amyloid plaque level in the subject is about 11 centiloids or lower as measured by one PET imaging.

In a particular embodiment, the subject is administered three first doses of 700 mg of the antibody of the present invention wherein each first dose is administered once every four weeks and then one or more second doses of 1400 mg of the antibody is administered wherein each second dose is administered once every four weeks until the amyloid plaque level in the patient is about 25 centiloids or lower.

In other embodiments, the subject is administered three first doses of 700 mg of the antibody of the present invention wherein each first dose is administered once every four weeks and then second doses of 1400 mg of the antibody is administered wherein each second dose is administered once every four weeks until amyloid plaque level in the patient is about 25 centiloids or lower for two consecutive PET imaging scans or about 11 centiloids or lower for one PET imaging scan. In some embodiments, the two consecutive PET imaging scans are at least 6 months apart.

In some embodiments, the subject is given no anti-Aβ antibody doses after amyloid plaque level in the patient is about 25 centiloids or lower for two consecutive PET imaging scans or about 11 centiloids or lower for one PET imaging scan. In some embodiments, the two consecutive PET imaging scans are at least 6 months apart.

In some embodiments, the subject may be given one or more 700 mg doses of anti-Aβ antibody after amyloid plaque level in the patient is about 25 centiloids or lower for two consecutive PET imaging scans or about 11 centiloids or lower for one PET imaging scan.

In some embodiments, the antibody of the present invention is administered to the subject until there is about 25 to about 150 centiloids reduction in amyloid plaque in the brain of the subject. See, e.g., Klunk et al., “The Centiloid Project: Standardizing Quantitative Amyloid Plaque Estimation by PET,” Alzheimer's & Dementia 11.1: 1-15 (2015) and Navitsky et al., “Standardization of Amyloid Quantitation with Florbetapir Standardized Uptake Value Ratios to the Centiloid Scale,” Alzheimer's & Dementia 14.12: 1565-1571 (2018), which are hereby incorporated by reference in their entireties.

In some embodiments, the antibody of the present invention is administered to the subject until there is about 50 to about 150 centiloids reduction in Aβ deposit in the brain of the subject. In some embodiments, the antibody of the present invention is administered to the subject until there is about 25, about 30, about 40, about 50, about 60, about 70, about 80, about 90, about 100, about 110, about 120, about 130, about 140 or about 150 centiloids reduction in Aβ deposit in the brain of the subject. In some embodiments, the antibody of the present invention is administered to the subject until there is about 50 centiloid reduction in Aβ deposits in the brain of the subject. In some embodiments, the antibody of the present invention is administered to the subject until there is about 60 centiloid reduction in Aβ deposits in the brain of the subject. In some embodiments, the antibody of the present invention is administered to the subject until there is about 70 centiloid reduction in Aβ deposits in the brain of the subject. In some embodiments, the antibody of the present invention is administered to the subject until there is about 80 centiloid reduction in Aβ deposits in the brain of the subject. In some embodiments, the antibody of the present invention is administered to the subject until there is about 84 centiloid reduction in Aβ deposits in the brain of the subject. In some embodiments, the antibody of the present invention is administered to the subject until there is about 90 centiloid reduction in Aβ deposits in the brain of the subject. In some embodiments, the antibody of the present invention is administered to the subject until there is about 100 centiloid reduction in Aβ deposits in the brain of the subject. In some embodiments, the antibody of the present invention is administered to the subject until there is about 110 centiloid reduction in Aβ deposits in the brain of the subject. In some embodiments, the antibody of the present invention is administered to the subject until there is about 120 centiloid reduction in Aβ deposits in the brain of the subject. In some embodiments, the antibody of the present invention is administered to the subject until there is about 130 centiloid reduction in Aβ deposits in the brain of the subject. In some embodiments, the antibody of the present invention is administered to the subject until there is about 140 centiloid reduction in Aβ deposits in the brain of the subject. In some embodiments, the antibody of the present invention is administered to the subject until there is about 150 centiloid reduction in Aβ deposits in the brain of the subject.

In some embodiments, the antibody of the present invention is administered to the subject until there is an average of about 25 to about 100 centiloids reduction in Aβ deposit in the brain of the subject. In some embodiments, the antibody of the present invention is administered to the subject until there is an average of about 50 to about 100 centiloids reduction in Aβ deposit in the brain of the subject. In some embodiments, the antibody of the present invention is administered to the subject until there is an average of about 10, about 20, about 30, about 40, about 50, about 60, about 70, about 80, about 84, about 90, about 100 centiloids reduction in Aβ deposit in the brain of the subject. In some embodiments, the antibody of the present invention is administered to the subject until there is an average of about 50 centiloid reduction in Aβ deposits in the brain of the subject. In some embodiments, the antibody of the present invention is administered to the subject until there is an average of about 60 centiloid reduction in Aβ deposits in the brain of the subject. In some embodiments, the antibody of the present invention is administered to the subject until there is an average of about 70 centiloid reduction in Aβ deposits in the brain of the subject. In some embodiments, the antibody of the present invention is administered to the subject until there is an average of about 80 centiloid reduction in Aβ deposits in the brain of the subject. In some embodiments, the antibody of the present invention is administered to the subject until there is an average of about 84 centiloid reduction in Aβ deposits in the brain of the subject. In some embodiments, the antibody of the present invention is administered to the subject until there is an average of about 90 centiloid reduction in Aβ deposits in the brain of the subject. In some embodiments, the antibody of the present invention is administered to the subject until there is an average of about 100 centiloid reduction in Aβ deposits in the brain of the subject.

In some embodiments, the second dose of the antibody of the present invention is administered to the subject until there is about 25 to about 150 centiloids reduction in Aβ deposit in the brain of the subject. In some embodiments, the second dose of the antibody of the present invention is administered to the subject until there is about 50 to about 150 centiloids reduction in Aβ deposit in the brain of the subject. In some embodiments, the second dose of the antibody of the present invention is administered to the subject until there is about 25, about 30, about 40, about 50, about 60, about 70, about 80, about 84, about 90, about 100, about 110, about 120, about 130, about 140 or about 150 centiloids reduction in Aβ deposit in the brain of the subject. In some embodiments, the second dose of the antibody of the present invention is administered to the subject until there is about 50 centiloid reduction in Aβ deposits in the brain of the subject. In some embodiments, the second dose of the antibody of the present invention is administered to the subject until there is about 60 centiloid reduction in Aβ deposits in the brain of the subject. In some embodiments, the second dose of the antibody of the present invention is administered to the subject until there is about 70 centiloid reduction in Aβ deposits in the brain of the subject. In some embodiments, the second dose of the antibody of the present invention is administered to the subject until there is about 80 centiloid reduction in Aβ deposits in the brain of the subject. In some embodiments, the second dose of the antibody of the present invention is administered to the subject until there is about 84 centiloid reduction in Aβ deposits in the brain of the subject. In some embodiments, the second dose of the antibody of the present invention is administered to the subject until there is about 90 centiloid reduction in Aβ deposits in the brain of the subject. In some embodiments, the second dose of the antibody of the present invention is administered to the subject until there is about 100 centiloid reduction in Aβ deposits in the brain of the subject. In some embodiments, the second dose of the antibody of the present invention is administered to the subject until there is about 110 centiloid reduction in Aβ deposits in the brain of the subject. In some embodiments, the second dose of the antibody of the present invention is administered to the subject until there is about 120 centiloid reduction in Aβ deposits in the brain of the subject. In some embodiments, the second dose of the antibody of the present invention is administered to the subject until there is about 130 centiloid reduction in Aβ deposits in the brain of the subject. In some embodiments, the second dose of the antibody of the present invention is administered to the subject until there is about 140 centiloid reduction in Aβ deposits in the brain of the subject. In some embodiments, the second dose of the antibody of the present invention is administered to the subject until there is about 150 centiloid reduction in Aβ deposits in the brain of the subject.

In some embodiments, the second dose of the antibody of the present invention is administered to the subject until there is an average of about 25 to about 100 centiloids reduction in Aβ deposit in the brain of the subject. In some embodiments, the second dose of the antibody of the present invention is administered to the subject until there is an average of about 50 to about 100 centiloids reduction in Aβ deposit in the brain of the subject. In some embodiments, the second dose of the antibody of the present invention is administered to the subject until there is an average of about 25, about 30, about 40, about 50, about 60, about 70, about 80, about 84, about 90, about 100 centiloids reduction in Aβ deposit in the brain of the subject. In some embodiments, the second dose of the antibody of the present invention is administered to the subject until there is an average of about 50 centiloid reduction in Aβ deposits in the brain of the subject. In some embodiments, the second dose of the antibody of the present invention is administered to the subject until there is an average of about 60 centiloid reduction in Aβ deposits in the brain of the subject. In some embodiments, the second dose of the antibody of the present invention is administered to the subject until there is an average of about 70 centiloid reduction in Aβ deposits in the brain of the subject. In some embodiments, the second dose of the antibody of the present invention is administered to the subject until there is an average of about 80 centiloid reduction in Aβ deposits in the brain of the subject. In some embodiments, the second dose of the antibody of the present invention is administered to the subject until there is an average of about 84 centiloid reduction in Aβ deposits in the brain of the subject. In some embodiments, the second dose of the antibody of the present invention is administered to the subject until there is an average of about 90 centiloid reduction in Aβ deposits in the brain of the subject. In some embodiments, the second dose of the antibody of the present invention is administered to the subject until there is an average of about 100 centiloid reduction in Aβ deposits in the brain of the subject.

In some embodiments, the antibodies, methods, dosing regimens, and/or uses of the present invention result in reduction of Aβ deposits in the brain of a human subject. In particular embodiments, the Aβ deposits are cleared or reduced by about 20-100% post treatment. In some embodiments, the antibody of the present invention is administered to the subject until there is about 20-100% reduction in Aβ deposits in the brain of the subject. In some embodiments, the antibody of the present invention is administered to the subject until the Aβ deposits in the brain of the subject are reduced by about 20%, about 25%, about 30%, about 35%, about 40%, about 45%, about 50%, about 75% or about 100%. In some embodiments, the antibody of the present invention is administered to the subject until there is about 20% reduction in Aβ deposits in the brain of the subject. In some embodiments, the antibody of the present invention is administered to the subject until there is about 25% reduction in Aβ deposits in the brain of the subject. In some embodiments, the antibody of the present invention is administered to the subject until there is about 30% reduction in Aβ deposits in the brain of the subject. In some embodiments, the antibody of the present invention is administered to the subject until there is about 35% reduction in Aβ deposits in the brain of the subject. In some embodiments, the antibody of the present invention is administered to the subject until there is about 40% reduction in Aβ deposits in the brain of the subject. In some embodiments, the antibody of the present invention is administered to the subject until there is about 50% reduction in Aβ deposits in the brain of the subject. In some embodiments, the antibody of the present invention is administered to the subject until there is about 75% reduction in Aβ deposits in the brain of the subject. In some embodiments, the antibody of the present invention is administered to the subject until there is about 100% reduction in Aβ deposits in the brain of the subject.

In some embodiments, the first dose and/or the second dose of antibody of the present invention are administered to the subject until the Aβ deposits in the brain of the subject are reduced by about 20-100%. In embodiments, the second doses of antibody of the present invention are administered to the subject until the Aβ deposits in the brain of the subject are reduced by about 20-100%. In some embodiments, the second doses of the antibody of the present invention are administered to the subject until the Aβ deposits in the brain of the subject are reduced by about 20%, about 25%, about 30%, about 35%, about 40%, about 45%, about 50%, about 75% or about 100%. In some embodiments, the second doses are administered to the subject until there is about 20% reduction in Aβ deposits in the brain of the subject. In some embodiments, the second doses are administered to the subject until there is about 25% reduction in Aβ deposits in the brain of the subject. In some embodiments, the second doses are administered to the subject until there is about 30% reduction in Aβ deposits in the brain of the subject. In some embodiments, the second doses are administered to the subject until there is about 35% reduction in Aβ deposits in the brain of the subject. In some embodiments, the second doses are administered to the subject until there is about 40% reduction in Aβ deposits in the brain of the subject. In some embodiments, the second doses are administered to the subject until there is about 50% reduction in Aβ deposits in the brain of the subject. In some embodiments, the second doses are administered to the subject until there is about 75% reduction in Aβ deposits in the brain of the subject. In some embodiments, the second doses are administered to the subject until there is about 100% reduction in Aβ deposits in the brain of the subject.

In some embodiments, the percentage reduction in Aβ deposits in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the centiloids reduction in Aβ deposits in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the average centiloids reduction in Aβ deposits in the brain of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the present invention results in about 15 to about 45 percent slowing of decline in the cognitive-functional composite endpoints from baseline. In some embodiments, the present invention results in about 15 to about 45 percent slowing of decline in the cognitive-functional composite endpoints from baseline over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks.

In some embodiments, the present invention results in about 15 to about 45 percent slowing of decline in the cognitive-functional composite endpoints from baseline over a duration of 76 weeks. In some embodiments, the slowing of decline in the cognitive-functional composite endpoints from baseline is provided from the MMRM model or the Bayesian Disease Progression Model (DPM). In some embodiments, the antibody of the present invention is administered to the subject till it reaches about 15 to about 45 percent slowing of decline in the cognitive-functional composite endpoints from baseline. In some embodiments, the first or the second dose of the present invention is administered to the subject till it reaches about 15 to about 45 percent slowing of decline in the cognitive-functional composite endpoints from baseline.

In some embodiments, the present invention results in about 15 to about 45 percent slowing of decline on the Integrated Alzheimer's Disease Rating Scale (iADRS) from baseline. In some embodiments, the present invention results in about 15 to about 45 percent slowing of decline on the Integrated Alzheimer's Disease Rating Scale from baseline over a duration of about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, about 72 weeks, or 76 weeks.

In some embodiments, the present invention results in about 20 percent, about 25 percent, about 30 percent, about 32 percent, about 35 percent, about 40 percent, or about 45 percent slowing of decline in the Integrated Alzheimer's Disease Rating Scale from baseline.

In some embodiments, the present invention results in about 15 to about 45 percent slowing of decline on the Integrated Alzheimer's Disease Rating Scale from baseline over a duration of 76 weeks. In a particular embodiment, the present invention results in about 32 percent slowing of decline on the Integrated Alzheimer's Disease Rating Scale from baseline over a duration of 76 weeks. In some embodiments, the antibody of the present invention is administered to the subject till it reaches about 15 to about 45 percent slowing of decline on the Integrated Alzheimer's Disease Rating Scale from baseline. In some embodiments, the first or the second dose of the present invention is administered to the subject till it reaches about 15 to about 45 percent slowing of decline on the Integrated Alzheimer's Disease Rating Scale from baseline.

In some embodiments, the cognitive functional composite endpoint, including iADRS, of the subject is measured at about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 56 weeks, about 60 weeks, about 64 weeks, about 68 weeks, or about 72 weeks.

In some embodiments, the antibody of the present invention can be administered in simultaneous, separate, or sequential combination with an effective amount of a symptomatic agent to treat Alzheimer's disease. Symptomatic agents can be selected from cholinesterase inhibitors (ChEIs) and/or a partial N-methyl-D-aspartate (NMDA) antagonists. In a preferred embodiment the agent is a ChEI. In another preferred embodiment the agent is a NMDA antagonist or a combination agent comprising a ChEI and NMDA antagonist.

In some embodiments, the disease characterized by Aβ deposit in the brain of the subject is selected from preclinical Alzheimer's disease, clinical AD, prodromal AD, mild AD, moderate AD, severe AD, Down's syndrome, clinical cerebral amyloid angiopathy, or pre-clinical cerebral amyloid angiopathy. In some embodiments, the subject is an early symptomatic AD patient. In some embodiments, the subject has prodromal AD and mild dementia due to AD. In some embodiments, the subject has mild cognitive impairment or mild dementia due to AD.

The present invention includes use of biomarkers of a disease characterized by Aβ deposits in the brain of a human subject, including Alzheimer's disease. Such biomarkers include, e.g., amyloid deposits, amyloid plaque, Aβ in CSF, Aβ in the plasma, brain tau deposition, tau in plasma, or tau in cerebrospinal fluid and their use in screening, diagnosis, treatment, or prevention. Non-limiting potential uses of such biomarkers include: 1) identification of subjects destined to become affected or who are in the “preclinical” stages of a disease; 2) reduction in disease heterogeneity in clinical trials or epidemiologic studies; 3) reflection of the natural history of disease encompassing the phases of induction, latency, and detection; and 4) target subjects for a clinical trial or for treatment/prevention of a disease.

In some embodiments, the biomarkers may be used to assess whether a subject can be treated using the antibodies, the dosing regimen, or the methods described herein. In some embodiments, the biomarkers may be used to assess whether a disease (as described herein) can be prevented in the subject using the antibodies, the dosing regimen, or the methods described herein. In some embodiments, the biomarkers can be used to assess whether a subject is responsive to treatment or prevention of a disease (as described herein) using the antibodies, the dosing regimen, or the methods described herein. In some embodiments, the biomarkers can be used to stratify or classify subjects into groups and to identify which group of subjects is responsive to treatment/prevention of diseases (as described herein) using the antibodies, the dosing regimen, or the methods described herein. In some embodiments, the biomarkers may be used to assess disease state of a subject and/or the duration for administration of the antibodies or doses thereof, as described herein, to the subject.

In some embodiments, the subject has a genetic mutation that causes autosomal-dominant Alzheimer's disease or at a higher risk for developing AD by virtue of carrying one or two APOE e4 alleles. In embodiments, the subject carries one or two APOE e4 alleles, i.e., the patient is heterozygous or homozygous.

In some embodiments, the subject has low to moderate tau burden or has been determined to have low to moderate tau burden. The subject may be characterized as having a low to moderate tau burden if the tau burden as measured by PET brain imaging (using, e.g., ¹⁸F flortaucipir) is from ≤1.10 standardized uptake value ratio (SUVr) to ≤1.46 SUVr. In some embodiments, the subject has low to moderate tau burden or has been determined to have low to moderate tau burden and carries one or two APOE e4 alleles.

In some embodiments, the subject has very low tau burden or has been determined to have very low tau burden. The subject may be characterized as having a very low tau burden if the tau burden as measured by PET brain imaging (using, e.g., ¹⁸F flortaucipir) is less than 1.10 SUVr. In some embodiments, the subject has very low tau burden or has been determined to have very low tau burden and carries one or two APOE e4 alleles.

In some embodiments, the subject has very low to moderate tau burden or has been determined to have very low tau to moderate tau burden. The subject may be characterized as having a very low to moderate tau burden if the tau burden as measured by PET brain imaging (using, e.g., ¹⁸F flortaucipir) is ≤1.46 SUVr. In some embodiments, the subject has very low to moderate tau burden or has been determined to have very low to moderate tau burden and carries one or two APOE e4 alleles.

In some embodiments, the subject does not have a high tau burden or has been determined to not have a high tau burden. In some embodiments, the human subject may be characterized as having a high tau burden if the tau burden as measured by PET brain imaging (using, e.g., ¹⁸F flortaucipir) is greater than 1.46 SUVr. In some embodiments, a subject with high tau is not administered the antibodies of the present invention. In some embodiments, the subject has does not have high tau burden or has been determined to not have a high tau burden and carries one or two APOE e4 alleles.

In some embodiments of the disclosed methods, the subject has a high tau burden. In some embodiments, the human subject may be characterized as having a high tau burden if the tau burden as measured by PET brain imaging (using, e.g., ¹⁸F flortaucipir) is greater than 1.46 SUVr. In some embodiments, the subject has high tau burden or has been determined to have a high tau burden and carries one or two APOE e4 alleles.

The subject having a high tau burden may be exhibiting a slow decline. A subject exhibiting a slow decline may be characterized as a subject that has not exhibited a decrease in the integrated Alzheimer's Disease Rating Scale (iADRS) of greater than about −20 over about the last 18 months. The iADRS is known in the art as a composite tool that combines scores from the AD Assessment Scale-Cognitive subscale (ADAS-Cog) and the AD Cooperative Study—instrumental Activities of Daily Living (ADCS-iADL). The iADRS may demonstrate acceptable psychometric properties, and the iADRS may be effective in capturing both disease progression and separation of placebo and active drug effect. In some embodiments, a subject with a high tau and a slow decline is administered the antibodies of the present invention. In other embodiments, a subject with a high tau and a fast decline is not administered the antibodies of the present invention. A subject exhibiting a fast decline may be characterized as a subject that has exhibited a decrease in the integrated Alzheimer's Disease Rating Scale (iADRS) of greater than about −20 over about the last 18 months.

According to embodiments of the present invention provided herein, the human subject has been determined to have slow decline by one of more of ADAS-Cog, iADL, CDR-SB, MMSE, APOE-4 genotyping and/or iADRS. In some embodiments, the human subject has been determined to have slow decline by iADRS. In some embodiments, iADRS has declined by less than 20. In some embodiments, iADRS has declined by less than 20 over a 6-month period. In some embodiments, iADRS has declined by less than 20 over a 12-month period. In some embodiments, iADRS has declined by less than 20 over an 18-month period. In some embodiments, iADRS has declined by less than 20 over a 24-month period. In some embodiments, the human subject has been determined to have slow decline by APOE-4 genotyping. In some embodiments, the human subject has been determined to be APOE-4 heterozygous. In some embodiments, the human subject has been determined to be APOE-4 homozygous negative. In some embodiments, the human subject has been determined to have slow decline by MMSE. In some embodiments, the human subject has been determined to have MMSE of above 27. In some embodiments, MMSE has declined by less than 3. In some embodiments, MMSE has declined by less than 3 over a 6-month period. In some embodiments, MMSE has declined by less than 3 over a 12-month period. In some embodiments, MMSE has declined by less than 3 over an 18-month period. In some embodiments, MMSE has declined by less than 3 over a 24-month period.

In some embodiments of the disclosed methods of treatment and prevention, the human subject has a tau burden as measured by PET brain imaging (using, e.g., ¹⁸F flortaucipir) that is less than about 1.46 SUVr, and the subject may be administered the antibodies of the present invention. In some embodiments of the disclosed methods of treatment and prevention, the human subject has a tau burden as measured by PET brain imaging (using, e.g., ¹⁸F flortaucipir) that is less than about 1.46 SUVr and has one or two alleles of APOE e4, and the subject may be administered the antibodies of the present invention. In other embodiments of the disclosed methods of treatment and prevention, the human subject has a tau burden as measured by PET brain imaging (using, e.g., ¹⁸F flortaucipir) that is less than about 1.27 SUVr, and the subject may be administered the antibodies of the present invention. In some embodiments of the disclosed methods of treatment and prevention, the human subject has a tau burden as measured by PET brain imaging (using, e.g., ¹⁸F flortaucipir) that is less than about 1.27 SUVr and has one or two alleles of APOE e4, and the subject may be administered the antibodies of the present invention.

In some embodiments, the anti-Aβ antibody, the dosing regimen, or the method described the present invention is efficacious in human subjects having very low to moderate tau. In some embodiments, the anti-Aβ antibody, the dosing regimen, or the method described the present invention is efficacious in human subjects having low to moderate tau. In some embodiments, the antibody of the present invention is most efficacious in human subjects having a tau level i) less than or equal to about 1.14 SUVr or ii) from about 1.14 SUVr to about 1.27 SUVr.

In some embodiments, the anti-Aβ antibody, the dosing regimen, or the method described the present invention is efficacious in human subjects having very low to moderate tau and carrying one or two APOE e4 alleles. In some embodiments, the anti-Aβ antibody, the dosing regimen, or the method described the present invention is efficacious in human subjects having low to moderate tau and carrying one or two APOE e4 alleles. In some embodiments, the antibody of the present invention is most efficacious in human subjects carrying one or two APOE e4 alleles and having a tau level i) less than or equal to about 1.14 SUVr or ii) from about 1.14 SUVr to about 1.27 SUVr.

The tau level of a human subject can be determined by techniques and methods familiar to the diagnosing physician or a person of ordinary skill in the art. In some embodiments, a human subject, who is suffering from a disease characterized by amyloid beta (Aβ) deposits, is determined to have very low to moderate tau, low to moderate tau, or no high tau using techniques and methods familiar to the diagnosing physician or a person of ordinary skill in the art. In some embodiments, such methods can also be used to prescreen, screen, diagnose, evaluate increase or reduction in brain tau burden, and/or to assess the progress achieved in the treatment or prevention of the diseases described herein. In some embodiments, the methods can also be used to stratify subjects into groups and/or to identify which group of subjects is responsive to treatment/prevention of a disease (as described herein) using the antibodies, the dosing regimen, or the methods described herein. In some embodiments, the methods or techniques used to determine/detect tau level of a human subject can be used for prescreening or screening subjects and determining which subjects are responsive to treatment/prevention of a disease (as described herein) using the antibodies, the dosing regimen, or the methods described herein.

For the purposes of the present invention, the tau level of a human subject can be determined using techniques or methods that, e.g., detect or quantitate i) brain tau deposition, ii) tau in plasma, or iii) tau in cerebrospinal fluid. In some embodiments, brain tau burden, tau in plasma, or tau in cerebrospinal fluid can be used to stratify subjects into groups and to identify which group of subjects is responsive to treatment/prevention of diseases (described herein) using the antibodies, the dosing regimen, or the methods described herein.

Tau levels in the brain of human subject can be determined using methods, such as, tau imaging with radiolabeled PET compounds (Leuzy et al., “Diagnostic Performance of RO948 F18 Tau Positron Emission Tomography in the Differentiation of Alzheimer Disease from Other Neurodegenerative Disorders,” JAMA Neurology 77.8:955-965 (2020); Ossenkoppele et al., “Discriminative Accuracy of [¹⁸F]-flortaucipir Positron Emission Tomography for Alzheimer Disease vs Other Neurodegenerative Disorders,” JAMA 320, 1151-1162, doi:10.1001/jama.2018.12917 (2018), which are hereby incorporated by reference in their entireties.

In some embodiments, the biomarker [¹⁸F]-florbtaucipir, which is a PET ligand, may be used for the purposes of the present invention. PET tau images can be, for example, quantitatively evaluated to estimate an SUVr (standardized uptake value ratio) by published methods (Pontecorvo et al., “A Multicentre Longitudinal Study of Flortaucipir (18F) in Normal Ageing, Mild Cognitive Impairment and Alzheimer's Disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and/or to visually evaluate patients, e.g., to determine whether the patient has an AD pattern (Fleisher et al., “Positron Emission Tomography Imaging With [¹⁸F]-flortaucipir and Postmortem Assessment of Alzheimer Disease Neuropathologic Changes,” JAMA Neurology 77:829-39 (2020), which is hereby incorporated by reference in its entirety). Lower SUVr values indicate less tau burden while higher SUVr values indicate a higher tau burden. In an embodiment, quantitative assessment by a flortaucipir scan is accomplished through an automated image processing pipeline as described in Southekal et al., “Flortaucipir F18 Quantitation Using Parametric Estimation of Reference Signal Intensity,” J. Nucl. Med 59:944-951 (2018), which is hereby incorporated by reference in its entirety. In some embodiments, counts within a specific target region of interest in the brain (e.g., multiblock barycentric discriminant analysis or MUBADA, see Devous et al, “Test-Retest Reproducibility for the Tau PET Imaging Agent Flortaucipir F18,” J. Nucl. Med 59:937-943 (2018), which is hereby incorporated by reference in its entirety) are compared with a reference region wherein the reference region is, e.g., whole cerebellum, (wholeCere), cerebellar GM (cereCrus), atlas-based white matter (atlasWM), subject-specific WM (ssWM, e.g., using parametric estimate of reference signal intensity (PERSI), see Southekal et al., “Flortaucipir F18 Quantitation Using Parametric Estimation of Reference Signal Intensity,” J. Nucl. Med. 59:944-951 (2018), which is hereby incorporated by reference in its entirety).

A preferred method of determining tau burden is a quantitative analysis reported as a standardized uptake value ratio (SUVr), which represents counts within a specific target region of interest in the brain (e.g., MUBADA,) when compared with a reference region (e.g., using PERSI).

In some embodiments, phosphorylated tau (P-tau; either phosphorylated at threonine 181 or 217) can be used to measure the tau load/burden for the purposes of the present invention (Barthelemy et al., “Cerebrospinal Fluid Phospho-tau T217 Outperforms T181 as a Biomarker for the Differential Diagnosis of Alzheimer's Disease and PET Amyloid-positive Patient Identification,” Alzheimer's Res. Ther. 12, 26, doi:10.1186/s13195-020-00596-4 (2020); Mattsson et al., “Aβ Deposition is Associated with Increases in Soluble and Phosphorylated Tau that Precede a Positive Tau PET in Alzheimer's Disease,” Science Advances 6, eaaz2387 (2020), which are hereby incorporated by reference their entireties). In a particular embodiment, antibodies directed against human tau phosphorylated at threonine at residue 217 can be used to measure the tau load/burden in a subject for the purposes of the present invention (see International Patent Application Publication No. WO 2020/242963, which is incorporated by reference in its entirety). The present invention includes, in some embodiments, the use of anti-tau antibodies disclosed in WO 2020/242963 to measure the tau load/burden in a subject. The anti-tau antibodies disclosed in WO 2020/242963 are directed against isoforms of human tau expressed in the CNS (e.g., recognizing the isoforms expressed in the CNS and not recognizing isoforms of human tau expressed exclusively outside the CNS). Such antibodies against isoforms of human tau expressed in the CNS can be used in a method of identifying/selecting a patient as one or more of: (i) having a disease disclosed herein; (ii) at risk for having a disease disclosed herein; (iii) in need of treatment for a disease disclosed herein; or (iv) in need of neurological imaging.

A subject is positive for amyloid deposits when amyloid is detected in the brain by methods such as, amyloid imaging with radiolabeled PET compounds or using a diagnostic that detects Aβ or a biomarker for Aβ. Exemplary methods that can be used in the present invention to measure the brain amyloid load/burden include, e.g., Florbetapir (Carpenter, et al., “The Use of the Exploratory IND in the Evaluation and Development of ¹⁸F-PET Radiopharmaceuticals for Amyloid Imaging in the Brain: A Review of One Company's Experience,” The Quarterly Journal of Nuclear Medicine and Molecular Imaging 53.4:387 (2009), which is hereby incorporated by reference in its entirety); Florbetaben (Syed et al., “[¹⁸F]Florbetaben: A Review in β-Amyloid PET Imaging in Cognitive Impairment,” CNS Drugs 29, 605-613 (2015), which is hereby incorporated by reference in its entirety); and Flutemetamol (Heurling et al., “Imaging β-amyloid Using [¹⁸F] Flutemetamol Positron Emission Tomography: From Dosimetry to Clinical Diagnosis,” European Journal of Nuclear Medicine and Molecular Imaging 43.2: 362-373 (2016), which is hereby incorporated by reference in its entirety).

[¹⁸F]-florbetapir can provide a qualitative and quantitative measurement of brain plaque load in patients, including patients with prodromal AD or mild AD dementia. For example, the absence of significant [¹⁸F]-florbetapir signal on a visual read indicates patients clinically manifesting cognitive impairment have sparse to no amyloid plaques. As such, [¹⁸F]-florbetapir also provides a confirmation of amyloid pathology. [¹⁸F]-Florbetapir PET also provides quantitative assessment of fibrillar amyloid plaque in the brain and, in some embodiments, can be used to assess amyloid plaque reductions from the brain by antibodies of the present invention.

Amyloid imaging with radiolabeled PET compounds can also be used to determine if Aβ deposit in the brain of a human patient is reduced or increased (e.g., to calculate the percentage reduction in Aβ deposit post treatment or to assess the progression of AD). A person of skill in the art can correlate the standardized uptake value ratio (SUVr) values obtained from amyloid imaging (with radiolabeled PET compounds) to calculate the % reduction in Aβ deposit in the brain of the patient before and after treatment. The SUVr values can be converted to standardized centiloid units, where 100 is average for AD and 0 is average for young controls, allowing comparability amongst amyloid PET tracers, and calculation of reduction according to centiloid units (Klunk et al., “The Centiloid Project: Standardizing Quantitative Amyloid Plaque Estimation by PET,” Alzheimer's & Dementia 11.1: 1-15 (2015) and Navitsky et al., “Standardization of Amyloid Quantitation with Florbetapir Standardized Uptake Value Ratios to the Centiloid Scale,” Alzheimer's & Dementia 14.12: 1565-1571 (2018), which are hereby incorporated by reference in their entireties). In some embodiments, the change in brain amyloid plaque deposition from baseline is measured by [¹⁸F]-florbetapir PET scan.

Cerebrospinal fluid or plasma-based analysis of β-amyloid can also be used to measure the amyloid load/burden for the purposes of the present invention. For example, Aβ42 can be used to measure brain amyloid (Palmqvist, S. et al., “Accuracy of Brain Amyloid Detection in Clinical Practice Using Cerebrospinal Fluid Beta-amyloid 42: a Cross-validation Study Against Amyloid Positron Emission Tomography. JAMA Neurol 71, 1282-1289 (2014), which is hereby incorporated by reference in its entirety). In some embodiments, the ratio of Aβ42/Aβ40 or Aβ42/Aβ38 can be used as a biomarker for amyloid beta (Janelidze et al., “CSF Abeta42/Abeta40 and Abeta42/Abeta38 Ratios: Better Diagnostic Markers of Alzheimer Disease,” Ann Clin Transl Neurol 3, 154-165 (2016), which is hereby incorporated by reference in its entirety).

In some embodiments, deposited brain amyloid plaque or Aβ in CSF or plasma can be used to stratify subjects into groups and to identify which group of subjects is responsive to treatment/prevention of a disease (as described herein) using the antibodies, the dosing regimen, or the methods described herein.

As used herein, “anti-Aβ antibody” refers to an antibody that binds to an epitope present on Aβ. In some embodiments, the anti-Aβ antibody binds to a soluble form of Aβ. In other embodiments, the anti-Aβ antibody binds to an insoluble form of Aβ, such as Aβ plaques. In some embodiments, the anti-Aβ antibody binds an epitope present in Aβ1-40 or Aβ1-42. In other embodiments, the anti-Aβ antibody binds an epitope present in a truncated form of Aβ1-40 or Aβ1-42, for example, a truncated form lacking 1-20 N terminal amino acids and/or lacking 1-20 C-terminal amino acids and optionally including an N-terminal pyroglutamate residue (e.g., N3pGlu Aβ). In other embodiments, the anti-Aβ antibody binds an epitope present in a fragment of Aβ1-40 or Aβ1-42 and having a length of about 5-20 amino acids and optionally comprising a N-terminal pyroglutamate. Anti-Aβ antibodies have been disclosed in the art. (See, e.g., U.S. Pat. Nos. 10,851,156; 10,738,109; 10,662,239; 10,654,917; 10,647,759; 10,603,367; 10,519,223; 10,494,425; 10,464,976; 10,112,991; 10,112,987; 10,035,847; 9,944,696; 9,939,452; 9,895,429; 9,834,598; 9,738,712; 9,585,956; 9,573,994; 9,382,312; 9,329,189; 9,309,309; 9,309,307; 9,272,031; 9,181,332; 9,176,150; 9,175,094; 9,146,244; 9,133,267; 9,125,846; 9,062,102; 9,051,364; 9,051,363; 8,916,165; 8,906,370; 8,906,367; 8,889,138; 8,796,439; 8,795,664; 8,710,193; 8,636,981; 8,614,299; 8,591,894; 8,507,206; 8,491,903; 8,470,321; 8,425,905; 8,420,093; 8,414,893; 8,398,978; 8,383,113; 8,337,848; 8,333,967; 8,323,654; 8,303,954; 8,268,973; 8,268,593; 8,246,954; 8,227,576; 8,222,002; 8,221,750; 8,173,127; 8,128,930; 8,128,928; 8,124,353; 8,124,076; 8,106,164; 8,105,594; 8,105,593; 8,025,878; 7,955,812; 7,939,075; 7,932,048; 7,927,594; 7,906,625; 7,902,328; 7,893,214; 7,892,545; 7,892,544; 7,871,615; 7,811,563; 7,807,165; 7,807,157; 7,790,856; 7,780,963; 7,772,375; 7,763,250; 7,763,249; 7,741,448; 7,731,962; 7,700,751; 7,625,560; 7,582,733; 7,575,880; 7,339,035; 7,320,790; 7,318,923; 7,256,273; 7,195,761; 7,189,819; 7,179,892; 7,122,374; 7,060,270; 6,815,175; 6,787,637; and 6,750,324; which are incorporated by reference in their entireties). Anti-Aβ antibodies also may include donanemab, aducanumab, bapineuzumab, GSK933776, solanezumab, lecanemab, crenezumab, ponezumab, and gantenerumab.

In some embodiments, the disclosed antibodies target N3pGlu Aβ (i.e., anti-N3pGlu Aβ antibody). The disclosed antibodies may selectively bind to N3pGlu Aβ peptide versus other Aβ peptides, such as peptides lacking the N-terminal pyroglutamate or the Aβ(1-40) peptide or Aβ(1-42) peptide. One of ordinary skill in the art will appreciate and recognize that “anti-N3pGlu Aβ antibody”, and several specific antibodies, including, “hE8L”, “B12L” and “R17L” are identified and disclosed (along with methods for making and using such antibodies) in U.S. Pat. No. 8,679,498 B2 (which is hereby incorporated by reference in its entirety). See, for example, Table 1 of U.S. Pat. No. 8,679,498 B2. Each of the antibodies disclosed in U.S. Pat. No. 8,679,498 B2, including “hE8L”, “B12L” and “R17L” antibodies, may be used as the anti-N3pGlu Aβ antibody of the present invention or in place of the anti-N3pGlu Aβ antibodies described in various aspects of the present invention. Other representative species of an anti-N3pGlu Aβ antibody include, but are not limited to, antibodies disclosed U.S. Pat. Nos. 8,961,972; 10,647,759; 9,944,696; WO 2010/009987A2; WO 2011/151076A2; WO 2012/136552A1 and equivalents thereto, e.g., under 35 U.S.C 112(f).

One of ordinary skill in the art will appreciate and recognize that “anti-N3pGlu Aβ antibody”, and several specific antibodies are identified and disclosed (along with methods for making and using such antibodies) in U.S. Pat. No. 8,961,972 (which is hereby incorporated by reference in its entirety); U.S. Pat. No. 10,647,759 (which is hereby incorporated by reference in its entirety); and U.S. Pat. No. 9,944,696 (which is hereby incorporated by reference in its entirety). Any of the anti-N3pGlu Aβ antibodies disclosed in the U.S. Pat. Nos. 8,961,972; 9,944,696; and 10,647,759 may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody VI”, “Antibody VII”, “Antibody VIII”, and “Antibody IX” are identified and disclosed (along with methods for making and using such antibodies) in WO2010/009987A2 (which is hereby incorporated by reference in its entirety). Each of these four antibodies (e.g., “Antibody VI”, “Antibody VII”, “Antibody VIII”, and “Antibody IX”) may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody X” and “Antibody XI” are identified and disclosed (along with methods for making and using such antibodies) in WO 2011/151076A2 (which is hereby incorporated by reference in its entirety). Each of these two antibodies (e.g., “Antibody X” and “Antibody XI”) may be used as the anti-N3pGlu Aβ antibody of the present invention or in place of the anti-N3pGlu Aβ antibodies described in various aspects of the present invention.

One of ordinary skill in the art will appreciate and recognize that “anti-N3pGlu Aβ antibody”, and several specific antibodies, including, “Antibody XII” and “Antibody XIII” are identified and disclosed (along with methods for making and using said antibodies) in WO 2012/136552A1 (which is hereby incorporated by reference in its entirety). Each of these two antibodies (e.g., “Antibody XII” and “Antibody XIII”) may be used as the anti-N3pGlu Aβ antibody of the present invention or in place of the anti-N3pGlu Aβ antibodies described in various aspects of the present invention.

As used herein, an “antibody” is an immunoglobulin molecule comprising two HC and two LC interconnected by disulfide bonds. The amino terminal portion of each LC and HC includes a variable region responsible for antigen recognition via the complementarity determining regions (CDRs) contained therein. The CDRs are interspersed with regions that are more conserved, termed framework regions. Assignment of amino acids to CDR domains within the LCVR and HCVR regions of the antibodies of the present invention is based on the following: Kabat numbering convention (Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)), and North numbering convention (North et al., A New Clustering of Antibody CDR Loop Conformations, Journal of Molecular Biology, 406:228-256 (2011)). Following the above method, the CDRs of the antibodies of the present invention were determined.

The antibodies of the present invention are monoclonal antibodies (“mAbs”). Monoclonal antibodies can be produced, for example, by hybridoma technologies, recombinant technologies, phage display technologies, synthetic technologies, e.g., CDR-grafting, or combinations of such or other technologies known in the art. The monoclonal antibodies of the present invention are human or humanized. Humanized antibodies can be engineered to contain one or more human framework regions (or substantially human framework regions) surrounding CDRs derived from a non-human antibody. Human framework germline sequences can be obtained from ImunoGeneTics (INGT) via their website, http://imgt.cines.fr, or from The Immunoglobulin FactsBook by Marie-Paule Lefranc and Gerard Lefranc, Academic 25 Press, 2001, ISBN 012441351. Techniques for generating human or humanized antibodies are well known in the art. In another embodiment of the present invention, the antibody, or the nucleic acid encoding the same, is provided in isolated form. As used herein, the term “isolated” refers to a protein, peptide or nucleic acid that is not found in nature and is free or substantially free from other macromolecular species found in a cellular environment. “Substantially free”, as used herein, means the protein, peptide or nucleic acid of interest comprises more than 80% (on a molar basis) of the macromolecular species present, preferably more than 90% and more preferably more than 95%.

The anti-Aβ antibody of the present invention is administered as a pharmaceutical composition. The pharmaceutical composition comprising an antibody of the present invention can be administered to a subject at risk for, or exhibiting, diseases or disorders as described herein by parental routes (e.g., subcutaneous, intravenous, intraperitoneal, intramuscular). Subcutaneous and intravenous routes are preferred. In some embodiment, the anti-N3pGlu Aβ antibody is administered by intravenous infusion.

The terms “treatment,” “treating” or “to treat” and the like include restraining, slowing, or stopping the progression or severity of an existing symptom, condition, disease, or disorder in a subject. The term “subject” refers to a human.

The term “prevention” means prophylactic administration of the antibody of the present invention to an asymptomatic subject or a subject with pre-clinical Alzheimer's disease to prevent onset or progression of the disease.

The terms “disease characterized by deposition of Aβ” or a “disease characterized by Aβ deposits” are used interchangeably and refer to a disease that is pathologically characterized by Aβ deposits in the brain or in brain vasculature. This includes diseases such as Alzheimer's disease, Down's syndrome, and cerebral amyloid angiopathy. A clinical diagnosis, staging or progression of Alzheimer's disease can be readily determined by the attending diagnostician or health care professional, as one skilled in the art, by using known techniques and by observing results. This generally includes brain plaque imaging, mental or cognitive assessment (e.g., Clinical Dementia Rating—summary of boxes (CDR-SB), Mini-Mental State Exam (MMSE) or Alzheimer's Disease Assessment Scale-Cognitive (ADAS-Cog)) or functional assessment (e.g., Alzheimer's Disease Cooperative Study-Activities of Daily Living (ADCS-ADL). The cognitive and functional assessment can be used to determine changes in a patient's cognition (e.g., cognitive decline) and function (e.g., functional decline). “Clinical Alzheimer's disease” as used herein is a diagnosed stage of Alzheimer's disease. It includes conditions diagnosed as prodromal Alzheimer's disease, mild Alzheimer's disease, moderate Alzheimer's disease, and severe Alzheimer's disease. The term “pre-clinical Alzheimer's disease” is a stage that precedes clinical Alzheimer's disease, where measurable changes in biomarkers (such as CSF Aβ42 levels or deposited brain plaque by amyloid PET) indicate the earliest signs of a patient with Alzheimer's pathology, progressing to clinical Alzheimer's disease. This is usually before symptoms such as memory loss and confusion are noticeable. Pre-clinical Alzheimer's disease also includes pre-symptomatic autosomal dominant carriers, as well as patients with higher risk for developing AD by virtue of carrying one or two APOE e4 alleles.

A reduction or slowing of cognitive decline can be measured by cognitive assessments such as Clinical Dementia Rating—summary of boxes, Mini-Mental State Exam or Alzheimer's Disease Assessment Scale-Cognitive. A reduction or slowing of functional decline can be measured by functional assessments such as ADCS-ADL.

As used herein, “mg/kg” means an amount, in milligrams, of antibody or drug administered to a subject based on his or her bodyweight in kilograms. A dose is given at one time. For example, a 10 mg/kg dose of antibody for a subject weighing 70 kg would be a single 700 mg dose of antibody given in a single administration. Similarly, a 20 mg/kg dose of antibody for a subject weighing 70 kg would be a 1400 mg dose of antibody given at a single administration.

As used herein, a human subject has “very low tau” burden if the tau burden is less than 1.10 SUVr (<1.10 SUVr) using ¹⁸F-flortaucipir based quantitative analysis where quantitative analysis refers to calculation of SUVr and SUVr represents counts within a specific target region of interest in the brain (multiblock barycentric discriminant analysis or MUBADA, see Devous et al, “Test-Retest Reproducibility for the Tau PET Imaging Agent Flortaucipir F18,” J. Nucl. Med. 59:937-943 (2018)) when compared with a reference region (parametric estimate of reference signal intensity or PERSI, see, Southekal et al., “Flortaucipir F 18 Quantitation Using Parametric Estimation of Reference Signal Intensity,” J. Nucl. Med 59:944-951 (2018)).

As used herein, a human subject has “very low tau to moderate tau” burden if the tau burden is less than or equal to 1.46 SUVr (i.e., ≤1.46 SUVr) using 18F-flortaucipir based quantitative analysis where quantitative analysis refers to calculation of SUVr and SUVr represents counts within a specific target region of interest in the brain (MUBADA, see Devous et al, “Test-Retest Reproducibility for the Tau PET Imaging Agent Flortaucipir F18,” J. Nucl. Med. 59:937-943 (2018)) when compared with a reference region (PERSI, see, Southekal et al., “Flortaucipir F 18 Quantitation Using Parametric Estimation of Reference Signal Intensity,” J. Nucl. Med 59:944-951 (2018)).

As used herein, a human subject has “low tau to moderate tau” burden if the tau burden is from greater than or equal to 1.10 to less than or equal to 1.46 (i.e., 1.10 SUVr to 1.46 SUVr) using ¹⁸F-flortaucipir based quantitative analysis where quantitative analysis refers to calculation of SUVr and SUVr represents counts within a specific target region of interest in the brain (MIUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med. 59:944-951 (2018)). “Low tau to moderate tau” burden can also be referred to as “intermediate” tau burden.

As used herein, a human subject has “high tau” burden if the tau burden is greater than 1.46 SUVr (i.e., >1.46 SUVr) using ¹⁸F-flortaucipir based quantitative analysis where quantitative analysis refers to calculation of SUVr and SUVr represents counts within a specific target region of interest in the brain (MIUBADA, see Devous et al, “Test-Retest Reproducibility for the Tau PET Imaging Agent Flortaucipir F18,” J. Nucl. Med 59:937-943 (2018)) when compared with a reference region (PERSI, see, Southekal et al., “Flortaucipir F 18 Quantitation Using Parametric Estimation of Reference Signal Intensity,” J. Nucl. Med. 59:944-951 (2018)).

As used herein, a human subject is exhibiting a slow decline if the human subject has not exhibited a decrease in the integrated Alzheimer's Disease Rating Scale (iADRS) of more than about −20 over about the last 18 months. A human subject is exhibiting a fast decline if the human subject has exhibited a decrease in the iADRS of more than about −20 over about the last 18 months.

As used herein, the term “about” means up to +10% unless the meaning of the term “about” differs from this meaning in view of the context of its use.

The terms “human subject” and “patient” are used interchangeably in the present disclosure.

As used herein, “methods of treatment” are equally applicable to use of a composition for treating the diseases or disorders described herein and/or compositions for use and/or uses in the manufacture of a medicaments for treating the diseases or disorders described herein.

The following Examples further illustrate the present invention. It should be understood however, that the Examples are set forth by way of illustration and not limitation, and that various modifications may be made by one of ordinary skill in the art.

### EXAMPLES

### Example 1: Expression and Purification of Engineered N3pGlu Aβ Antibodies

Antibodies to N3pGlu Aβ were selected as exemplary antibodies for this example. Antibodies to N3pGlu Aβ are known in the art. For example, U.S. Pat. Nos. 8,679,498 and 8,961,972 (which are hereby incorporated by reference in their entireties) disclose anti-N3pGlu Aβ antibodies, method of making the antibodies, antibody formulations, and methods of treating diseases, such as, Alzheimer's disease with the antibodies.

An exemplary method of expressing and purifying the anti-N3pGlu Aβ antibodies of the present invention is follows. An appropriate host cell, such as HEK 293 EBNA or CHO, is either transiently or stably transfected with an expression system for secreting antibodies using an optimal predetermined HC:LC vector ratio or a single vector system encoding both HC and LC. Clarified media, into which the antibody has been secreted, is purified using any of many commonly used techniques. For example, the medium may be conveniently applied to a Protein A or G Sepharose FF column that has been equilibrated with a compatible buffer, such as phosphate buffered saline (pH 7.4). The column is washed to remove nonspecific binding components. The bound antibody is eluted, for example, by pH gradient (such as 0.1 M sodium phosphate buffer pH 6.8 to 0.1 M sodium citrate buffer (pH 2.5). Antibody fractions are detected, such as by SDS-PAGE, and are pooled. Further purification is optional, depending on the intended use. The antibody may be concentrated and/or sterile filtered using common techniques. Soluble aggregate and multimers may be effectively removed by common techniques, including size exclusion, hydrophobic interaction, ion exchange, or hydroxyapatite chromatography. The purity of the antibody after these chromatography steps is greater than 99%. The product may be immediately frozen at −70° C. or may be lyophilized.

### Example 2: Assessment of Safety, Tolerability, and Efficacy of Anti-N3pGlu Aβ Antibody

Donanemab was selected as an exemplary antibody for this example. A multicenter, randomized, double-blind, placebo-controlled, Phase 2 clinical study (NCT03367403; clinicaltrials.gov) was designed to evaluate the safety and efficacy of an N3pGlu Aβ antibody (also referred to herein as Donanemab) in AD subjects with early symptomatic AD (prodromal AD and mild dementia due to AD). This phase 2 study assessed, including other things, whether removal of existing amyloid plaque can slow the progression of disease as determined by clinical measures and biomarkers of disease pathology and neurodegeneration over up to 72 weeks of treatment.

This study was a 133-week study and included a screening period of up to 9 weeks, a treatment period of up to 72 weeks with final evaluations occurring 4 weeks later at Week 76, and a 48-week immunogenicity and a safety follow-up period.

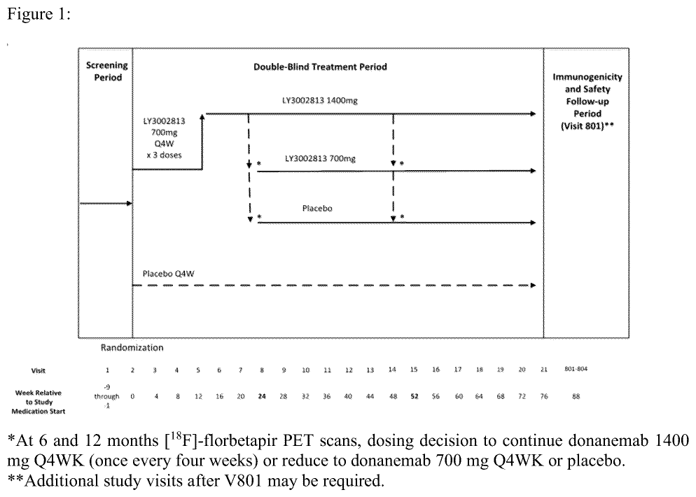

FIG. 1 illustrates the study design for clinical protocol.

Treatment Arms and Duration: Approximately 1497 patients were screened and approximately 266 were randomized. Patients received the following treatments (dosing) for up to 72 weeks:
Donanemab: intravenous donanemab (700 mg Q4WK for the first 3 doses, then 1400 mg Q4WK) for up to 72 weeks; or
Placebo: intravenous placebo Q4WK for up to 72 weeks.

### Primary and Secondary Endpoints:

The primary endpoint for this study was:
Change in cognition and function as measured by the change in integrated Alzheimer's Disease Rating Scale (iADRS) score from baseline to 18 months.
   The secondary endpoints for this study were:
Change in cognition from baseline to 18 months as measured by: the change in ADAS-Cog₁₃ score, the change in Clinical Dementia Rating Scale Sum of Boxes score (CDR-SB), the change in Mini-Mental State Examination score (MMSE), and the change in Alzheimer's Disease Cooperative Study-instrumental Activities of Daily Living scale (ADCS-iADL) score.
Change in brain amyloid plaque deposition from baseline through 18 months as measured by [¹⁸F]-florbetapir PET scan.
Change in brain tau deposition from baseline to 18 months as measured by [¹⁸F]-florbetapir PET scan.
Change in volumetric MRI measures from baseline to 18 months.

### Safety Endpoints:

The safety endpoints for this study are:
Standard safety assessments: spontaneously reported adverse events (AEs), clinical laboratory tests, vital sign and body weight measurements, 12-lead electrocardiograms (ECGs), physical and neurological examinations
MRI (amyloid-related imaging abnormalities [ARIAs] and emergent radiological findings)
Columbia Suicide Severity Rating Scale (C-SSRS)
   Statistical Analysis: All efficacy analyses will follow the intent-to-treat (ITT) principle unless otherwise specified. An ITT analysis is an analysis of data by the groups to which subjects are assigned by random allocation, even if the subject does not take the assigned treatment, does not receive the correct treatment, or otherwise does not follow the protocol. Unless otherwise noted, all pairwise tests of treatment effects was conducted at a 2-sided alpha (α) level of 0.05; 2-sided confidence intervals (CIs) are displayed with a 95% confidence level.
   Efficacy: The primary objective of this study was to test the hypothesis that intravenous infusion of donanemab will slow the cognitive and/or functional decline of AD as measured by the composite measure iADRS compared with placebo in patients with early symptomatic AD. The change from baseline score on the iADRS at each scheduled postbaseline visit during the treatment period was analyzed using an MMRM model, which includes the following terms: baseline score, pooled investigator, treatment, visit, treatment-by-visit interaction, baseline-by-visit interaction, concomitant acetylcholine esterase inhibitor (AChEI) and/or memantine use at baseline (yes/no), and age at baseline. The primary time point for treatment comparison was at the end of the double-blind treatment period (Week 76). The treatment group contrast in least-squares mean progression and its associated p-value and 95% CI was calculated for the treatment comparison of donanemab vs. placebo. In addition, Bayesian posterior probability of the active treatment arm being superior to placebo by at least a margin of interest (25% slowing of placebo progression) was calculated.

Change from baseline at each scheduled postbaseline visit during the treatment period in secondary efficacy outcomes, including ADAS-Cog₁₃, ADCS-iADL, CDR-SB, and MMSE, is analyzed using the same MMRM model described for the primary analysis.

Safety: Safety is assessed by summarizing and analyzing AEs, laboratory analytes, vital signs, MRI scans, ECGs, immunogenicity during the double-blind treatment period.
Pharmacokinetics Pharmacodynamics: Pharmacokinetic or pharmacodynamic (PK/PD) relationships between plasma donanemab concentration and SUVr, cognitive endpoints, ARIA incidence rate or other markers of PD activity was explored graphically. The relationship between the presence of antibodies to donanemab and PK, PD, safety and/or efficacy may be assessed graphically. If warranted, additional analysis may be explored to evaluate potential interactions for anti-drug antibodies, PD, and other endpoints (PET scan, ARIA-E, etc.). Additional modeling may be performed based on the results of the graphical analyses.
Dosing and Dose Justification: Donanemab (700 mg or 1400 mg) is administered every 4 weeks as an IV infusion of approximately 140 mL over a minimum of 30 minutes. Donanemab doses of 700 mg and 1400 mg administered intravenously once every 4 weeks are selected based on current preclinical pharmacology and toxicology data and clinical PK, PD, and safety data. Prior and ongoing exposures include 0.1 mg/kg, 0.3 mg/kg, 1 mg/kg, 3 mg/kg, 10 mg/kg, 20 mg/kg, and 40 mg/kg in single and/or multi-dose dosing schedules. Data from Study AACC (NCT01837641, clinicaltrials.gov) suggests that PK of donanemab is linear when the dose is not less than 10 mg/kg. Mean half-life is about 9-11 days when dose is ≥10 mg/kg, so minimal accumulation in plasma PK is predicted for 700 mg and 1400 mg Q4 week IV dosing. High levels of [¹⁸F]-florbetapir PET signal reductions are observed with a single dose of 20 mg/kg and are comparable to [¹⁸F]-florbetapir PET reductions seen with a 10 mg/kg Q2 week dosing schedule at 3 months. Based on this as well as decreased patient burden with an every 4 week dosing schedule compared with an every 2 week dosing schedule and comparable safety, 1400 mg Q4 week dosing is selected as the highest dose regimen for robust amyloid plaque lowering. The lowest rate of ARIA-E is been observed with 10 mg/kg monthly dosing. For this reason, a titration schedule (700 mg Q4 week for the first 3 doses, then 1400 mg Q4 week) is proposed to reduce ARIA incidence while allowing patients to achieve high PD effects. In addition, dose reduction rules have been established for incident ARIA-E.
Inclusion Criteria: Patients, including both men and women, 60 to 85 years of age, inclusive, at the time of informed consent were eligible for enrollment in the study. The patients may exhibit gradual and progressive change in memory function reported by patients or study partners (informants) for ≥6 months. In some instances, the patient may have an MMSE score of 20 to 28 (inclusive) at Visit 1 or an acceptable historical [¹⁸F]-flortaucipir PET scan within 6 months prior to Visit 1 that meets central read criterion. The patients may also meet [¹⁸F]-flortaucipir scan (central read) criteria and/or [¹⁸F]-florbetapir scan (central read) criteria.
Exclusion Criteria: Patients are excluded from study enrollment if they meet any of the following criteria: have a Modified Hachinski Ischemia Scale (MHIS; Hachinski et al. 1975) score of ≥4; lack, in the investigator's opinion, of adequate premorbid literacy, adequate vision, or adequate hearing to complete the required psychometric tests; significant neurological disease affecting the central nervous system (CNS), other than AD, that may affect cognition or ability to complete the study, including but not limited to, other dementias, serious infection of the brain, Parkinson's disease, multiple concussions, or epilepsy or recurrent seizures (except febrile childhood seizures); current serious or unstable illnesses including cardiovascular, hepatic, renal, gastroenterologic, respiratory, endocrinologic, neurologic (other than AD), psychiatric, immunologic, or hematologic disease and other conditions that, in the investigator's opinion, could interfere with the analyses in this study; or have a life expectancy of <24 months; have a history of cancer within the last 5 years, with the exception of non-metastatic basal and/or squamous cell carcinoma of the skin, in situ cervical cancer, non-progressive prostate cancer, or other cancers with low risk of recurrence or spread; patients with any current primary psychiatric diagnosis other than AD if, in the judgment of the investigator, the psychiatric disorder or symptom is likely to confound interpretation of drug effect, affect cognitive assessment, or affect the patient's ability to complete the study; patients with history of schizophrenia or other chronic psychosis; have a history of long QT syndrome; are clinically judged by the investigator to be at serious risk for suicide as assessed by medical history, examination, or the C-SSRS; history of alcohol or drug use disorder (except tobacco use disorder) within 2 years before the screening visit; have a history of clinically significant multiple or severe drug allergies, or severe post-treatment hypersensitivity reactions (including but not limited to erythema multiforme major, linear immunoglobulin A dermatosis, toxic epidermal necrolysis, and/or exfoliative dermatitis); or have a known positive serologic findings for human immunodeficiency virus (HIV) antibodies. Local laws and regulations may apply to whether testing is required; have any clinically important abnormality at screening, as determined by investigator, in physical or neurological examination, vital signs, ECG, or clinical laboratory test results that could be detrimental to the patient, could compromise the study, or show evidence of other etiologies for dementia; screening MRI which shows evidence of significant abnormality that would suggest another potential etiology for progressive dementia or a clinically significant finding that may impact the patient's ability to safely participate in the study; have any contraindications for MRI, including claustrophobia or the presence of contraindicated metal (ferromagnetic) implants/cardiac pacemaker; have a centrally read MRI demonstrating presence of ARIA-E, >4 cerebral microhemorrhages, more than 1 area of superficial siderosis, any macro hemorrhage or severe white matter disease; an average (ECG in triplicate) corrected QT (QTcF) interval measurement >450 msec (men) or >470 msec (women) at screening (as determined at the investigational site; patients with a past history of Hepatitis B should have HBsAg testing at screening and are excluded if HBsAg is positive; patients with past history of Hepatitis C should have HCV RNA PCR testing at screening and are excluded if HCV RNA PCR is positive; calculated creatinine clearance <30 mL/min (Cockcroft-Gault formula; Cockcroft and Gault 1976) at screening; alanine transaminase (ALT)≥2× the upper limit of normal (ULN) of the performing laboratory, aspartate aminotransferase (AST)≥2× ULN, total bilirubin level (TBL)≥1.5× ULN, or alkaline phosphatase (ALP)≥1.5× ULN at screening; have received treatment with a stable dose of an AChEI and/or memantine for less than 2 months before randomization; changes in concomitant medications that could potentially affect cognition and their dosing should be stable for at least 1 month before screening, and between screening and randomization (does not apply to medications discontinued due to exclusions or with limited duration of use, such as antibiotics); current use of drugs known to significantly prolong the QT interval; have had prior treatment with a passive anti-amyloid immunotherapy <5 half-lives prior to randomization; have received active immunization against Aβ in any other study; have known allergies to donanemab, related compounds, or any components of the formulation; or history of significant atopy; have allergies to either monoclonal antibodies, diphenhydramine, epinephrine, or methylprednisolone; sensitivity to [¹⁸F]-florbetapir or [¹⁸F]-flortaucipir; contraindication to MRI; contraindication to PET; present or planned exposure to ionizing radiation that, in combination with the planned administration of study PET ligands, would result in a cumulative exposure that exceeds local recommended exposure limits.
Dosage Modification for ARIA-E: Donanemab dosage modifications are adjusted for the occurrence of ARIA-E in the following instances depicted in Table A. If a dosage reduction is required, the donanemab dose is reduced to the next lower dose (from 1400 mg to 700 mg or from 700 mg to placebo).

TABLE A
Donanemab Dosage Modifications for First Occurrence of ARIA-E

| ARIA-E | ARIA-E on MRI | | |
|---|---|---|---|
| SYMPTOMS | Mild | Moderate | Severe |
| No symptoms | Continue current | donanemab | Temporary |
| | dosing^{a} | Dose reduction^{a,b} | Discontinuation |
| | | | of donanemab |
| Mild | donanemab | Temporary | Temporary |
| | Dose reduction^{a,b} | Discontinuation | Discontinuation |
| | | of donanemab | of donanemab |
| Moderate | Temporary | Temporary | Temporary |
| | Discontinuation | Discontinuation | Discontinuation |
| | of donanemab | of donanemab | of donanemab |
| Severe | Temporary | Temporary | Temporary |
| | Discontinuation | Discontinuation | Discontinuation |
| | of donanemab | of donanemab | of donanemab |

^{a}Investigator may choose to temporarily discontinue donanemab after discussion with the sponsor.
^{b}If the patient has a second incidence of ARIA-E and has previously been dose reduced or temporarily discontinued from donanemab, then donanemab is permanently discontinued.

All Cases of ARIA-E will require unscheduled MRI scans every 4-6 weeks until ARIA-E has resolved.
Discontinuation from Study Treatment: Possible reasons leading to permanent discontinuation of study treatment: Subject Decision (the subject or the subject's designee; for example, legal guardian requests to discontinue investigational product) or discontinuation due to a hepatic event or liver test abnormality. Subjects who are discontinued from investigational product due to a hepatic event or liver test abnormality should have additional hepatic safety data collected via CRF/electronic data entry.

Discontinuation of the investigational product for abnormal liver tests is considered when a subject meets one of the following conditions: alanine aminotransferase (ALT) or aspartate aminotransferase (AST)>8× upper limit of normal (ULN); ALT or AST>5× ULN for more than 2 weeks; ALT or AST>3× ULN and total bilirubin level (TBL)>2× ULN or international normalized ratio (INR)>1.5; ALT or AST>3× ULN with the appearance of fatigue, nausea, vomiting, right upper-quadrant pain or tenderness, fever, rash, and/or eosinophilia (>5%); alkaline phosphatase (ALP)>3× ULN; ALP>2.5× ULN and TBL>2× ULN; or ALP>2.5× ULN with the appearance of fatigue, nausea, vomiting, right quadrant pain or tenderness, fever, rash, and/or eosinophilia (>5%).

In addition, subjects are discontinued from the investigational product in the following circumstances:
Treatment with donanemab is permanently discontinued in patients with:
   a second incidence of ARIA-E after a previous dose reduction or temporary discontinuation of donanemab;
   any increase in ARIA-H accompanied by clinically significant symptoms;
   >4 new microhemorrhages, >1 new area of superficial siderosis or significant worsening of pre-existing superficial siderosis, or any macro hemorrhage regardless of symptoms; or
   an ARIA-E event reported as a significant adverse event (SAE), regardless of severity of symptoms or MRI findings.
Treatment with donanemab will also be permanently discontinued in patients with:
   Prolonged acute infusion reaction (i.e., not responsive to medication such as antihistamines, nonsteroidal anti-inflammatory drugs, and/or narcotics and/or brief interruption of infusion); or
   Adverse event or clinically significant laboratory value, ECG result, physical examination finding, MRI finding (such as symptomatic ischemic stroke),
      Temporary Discontinuation from Donanemab Study Treatment Due to ARIA-E

Temporary discontinuation from donanemab treatment is allowed for ARIA-E if the ARIA-E meets the temporary discontinuation criteria shown in Table A. In cases of ARIA-E where the protocol indicates continued dosing or a dose reduction rather than temporary discontinuation, the administration of donanemab may be temporarily discontinued.

Donanemab may be restarted following a first incidence of ARIA-E if, e.g., dosing is temporarily discontinued due to ARIA-E and there is complete resolution of symptoms and radiologic findings within 16 weeks after the temporary drug discontinuation. If ARIA-E symptoms and radiologic findings have not completely resolved within 16 weeks, then the patient is permanently discontinued from donanemab treatment.

Study drug may be restarted at either 700 mg or placebo, double blinded, depending on the original study arm to which the patient is randomized. An unscheduled safety MRI scan is required 4-6 weeks after dose restarts.

Efficacy Assessments: Cognitive and functional testing is administered using an eCOA tablet. The audio voice recordings of the rater's questions and the patient's and study partner's responses will also be collected via the eCOA tablet during administration of the cognitive and functional testing for central monitoring of rater scale administration. Cognitive and functional testing for each patient should be performed at approximately the same time on each day that testing occurs to reduce potential variability. Note that the ADAS-Cog and MMSE must be administered by a different rater than the ADCS-ADL and CDR. These 2 raters should continue doing the same scale with the same patient throughout the study. If possible, each assessment should be performed on a given patient by the same rater at each visit. The principal investigator (PI) has the responsibility of selecting the raters who will administer the instruments at the site if all training requirements have been met by those raters.

When administered, cognitive and functional testing should be performed first, before medical procedures that could be stressful for the patient (e.g., blood draws). Note that some procedures (MRI, [¹⁸F]-flortaucipir PET tau imaging, [¹⁸F]-florbetapir PET amyloid imaging) can be conducted on other days within the visit window.

### Primary Efficacy Assessments:

Integrated Alzheimer's Disease Rating Scale (iADRS; Wessels et al., “A Combined Measure of Cognition and Function for Clinical Trials: The Integrated Alzheimer's Disease Rating Scale (iADRS),” J Prev Alzheimers Dis. 2(4):227-241 (2015), which is hereby incorporated by reference in its entirety). The iADRS represents a composite that was developed using both a theory-driven approach (incorporating measures of both cognition and function) and a data-mining approach (identifying the most sensitive combination of scales through analysis of data from the Alzheimer's Disease Neuroimaging Initiative). The iADRS is a simple linear combination of scores from 2 well-established, therapeutically sensitive, widely accepted measures in AD, the ADAS-Cog₁₃ and the ADCS-iADL, measuring the core domains of AD. All items of these 2 scales are included without additional weighting of items, yielding face validity and ease of interpretation of the composite relative to its components. The iADRS score is derived from the ADAS-Cog₁₃ and the ADCS-iADL and is the primary efficacy measure. The ADAS-Cog₁₃ and the ADCS-ADL are the actual scales administered to patients.
Secondary Efficacy Assessments: Additional clinical outcome measures should be administered in the same order at every visit, immediately following assessment of the ADAS-Cog₁₃. To minimize missing data, the rater should include each measure orally with the patient or study partner (as designated in instructions), recording responses appropriately. The same study partner should be used as the informant at all visits.
Alzheimer's Disease Assessment Scale Cognitive subscale: The ADAS-Cog₁₃ is a rater-administered instrument that was designed to assess the severity of the dysfunction in the cognitive and noncognitive behaviors characteristic of persons with AD (Rosen et al., “A New Rating Scale for Alzheimer's Disease,” Am J Psychiatry. 141(11):1356-1364 (1984), which is hereby incorporated by reference in its entirety). The ADAS-Cog₁₃ should be administered by the same rater from visit to visit to reduce potential variability. The cognitive subscale of the ADAS, the ADAS-Cog₁₃, consists of 13 items assessing areas of cognitive function most typically impaired in AD: orientation, verbal memory, language, praxis, delayed free recall, digit cancellation, and maze-completion measures (Mohs et al., “Development of Cognitive Instruments for Use in Clinical Trials of Antidementia Drugs: Additions to the Alzheimer's Disease Assessment Scale that Broaden its Scope,” The Alzheimer's Disease Cooperative Study. Alzheimer Dis Assoc Disord. 11(Suppl 2):S13-S21 (1997), which is hereby incorporated by reference in its entirety). The ADAS-Cog₁₃ allows better discrimination of differences among mild patients than the ADAS-Cog11 and is included as a secondary outcome. The ADAS-Cog₁₃ scale ranges from 0 to 85, with higher scores indicating greater disease severity.
Alzheimer's Disease Cooperative Study—Activities of Daily Living Inventory: The ADCS-ADL is a 23-item inventory developed as a rater-administered questionnaire that is to be answered by the patient's study partner (Galasko et al., “An Inventory to Assess Activities of Daily Living for Clinical Trials in Alzheimer's Disease,” The Alzheimer's Disease Cooperative Study. Alzheimer Dis Assoc Disord. 1997; 11(Suppl 2):S33-S39; Galasko et al., “Galantamine Maintains Ability to Perform Activities of Daily Living in Patients with Alzheimer's Disease,” J Am Geriat Soc. 52(7):1070-1076 (2004), which are hereby incorporated by reference in their entireties). The ADCS-ADL should be administered by the same rater from visit to visit to reduce potential variability. The ADCS-ADL subset of items (items 7 to 23) for instrumental activities of daily living (ADCS-iADL) is used as a secondary efficacy measure. The focus in the early symptomatic AD population is on the instrumental activities of daily living (iADLs) rather than the basic activities of daily living (bADLs), which are thought to be affected in more severe stages of the disease. The range for the iADL score is 0 to 56, with lower scores indicating greater disease severity. For each of the specific items, the study partner is first asked if the patient attempted the ADL during the past 4 weeks. If the patient did attempt the ADL, the study partner is asked to rate the patient's performance level based on a set of performance descriptions. Scores for each item and the overall score for the tool are calculated. The range for the total ADCS-ADL score is 0 to 78, with higher scores indicating greater level of impairment. Separate scores for the bADLs (0 to 22) are also be computed.
Clinical Dementia Rating Scale: The CDR is a semi-structured interview performed with the patient and study partner (informant) that provides an index of global functioning (Berg et al., “Mild Senile Dementia of the Alzheimer's Type. 4. Evaluation of Intervention,” Ann Neurol. 31(3):242-249 (1992), which is hereby incorporated by reference in its entirety). The CDR should be administered by the same rater from visit to visit to reduce potential variability. The informant is queried about the patient's memory, orientation, judgment, and problem solving, community affairs, home and hobbies, and personal care. The patient's memory, orientation, judgment, and problem-solving ability are assessed. Higher scores indicate greater disease severity. By assigning a severity score for each of the 6 domains, a total score known as sum of boxes is obtained-hence the abbreviation, CDR-SB. The range for CDR-SB is from 0 to 18, higher values indicating greater impairment
Mini-Mental State Examination: The MMSE is a brief instrument used to assess cognitive function in patients (Folstein et al., “Mini-Mental State”. A Practical Method for Grading the Cognitive State of Patients for the Clinician,” J Psychiatr Res. 12(13):189-198 (1975), which is hereby incorporated by reference in its entirety). The MMSE should be administered by the same rater from visit to visit to reduce potential variability. The instrument is divided into 2 sections. The first section measures orientation, memory, and attention. The maximum score for the first section is 21. The second section tests the ability of the patient to name objects, follow verbal and written commands, write a sentence, and copy figures. The maximum score for the second section is 9. The range for the total MMSE score is 0 to 30, with lower scores indicating great level of impairment.
Biomarker Efficacy Measures (Double-Blind Period) [¹⁸F]-Florbetapir PET scan: Change in amyloid burden (as assessed by [¹⁸F]-florbetapir PET signal) is compared in donanemab- and placebo-treated patients for those patients who undergo [¹⁸F]-florbetapir PET scans at baseline, Week 52 [Visit 15], and Week 76 [Visit 21] or an early discontinuation visit (ED).
[⁸F]-Flortaucipir PET scan: Change in tau burden (as assessed by [¹⁸F]-flortaucipir PET signal) is compared in donanemab- and placebo-treated patients for those patients who undergo both baseline and endpoint (Visit 21 [Week 76] or ED) [¹⁸F]-flortaucipir scans.
Volumetric MR: Magnetic resonance imaging of the brain may be performed during visits 2-14. Donanemab- and placebo-treatment effects on volumetric MRI is assessed and compared to evaluate the loss of brain volume that occurs in AD patients.
Clearance of Amyloid Deposits: Clearance of amyloid deposits (as assessed by [¹⁸F]-florbetapir PET signal) is compared in donanemab- and placebo-treated patients for those patients who undergo baseline, Visit 8 (Week 24), Visit 15 (Week 52) and endpoint Visit 21 (Week 76), or ED [¹⁸F]-florbetapir PET scans.
Accumulation of Tau Deposits: Extent of accumulation of tau PHF deposits (as assessed by [¹⁸F]-flortaucipir PET signal) is compared in donanemab- and placebo-treated patients for those patients who undergo baseline and endpoint Visit 21 (Week 76), or ED [¹⁸F]-flortaucipir PET scans.
Biomarkers: Biomarker research is performed to address questions of relevance to drug disposition, target engagement, PD, mechanism of action, variability of patient response (including safety), and clinical outcome. Sample collection is incorporated into clinical studies to enable examination of these questions through measurement of biomolecules including deoxyribonucleic acid (DNA), ribonucleic acid (RNA), proteins, lipids, and other cellular elements. Serum, plasma, and whole blood RNA samples for biomarker research are collected during visits 2-14, where local regulations allow.

### Example 3: Results from Safety, Tolerability, and Efficacy Study

Donanemab was selected as an exemplary antibody for this example. This example provides the results obtained from safety, adverse events, and efficacy of donanemab in participants with early symptomatic AD. Enrolment was based on florbetapir and flortaucipir Positron Emission Tomography (PET) scan demonstrating tau and amyloid plaque pathology, respectively. Participants received either intravenous placebo or donanemab (700 mg for doses 1-3 and 1400 mg thereafter) every 4 weeks for up to 72 weeks. The primary outcome measure was change from baseline in the integrated AD Rating Scale (iADRS; range 0 to 144, lower indicating greater cognitive deficit and impairment of activities of daily living) at 76 weeks. Secondary outcome measures included Clinical Dementia Rating Scale Sum of Boxes (CDR-SB; range 0 to 18, higher indicating greater impairment), AD Assessment Scale-Cognitive (ADAS-Cog₁₃; range 0 to 85, higher indicating greater disease severity), AD Cooperative Study-instrumental Activities of Daily Living (ADCS-iADL; range 0 to 59, lower indicating greater impairment), Mini-Mental State Examination (MMSE; range 0 to 30, lower indicating greater impairment), amyloid and tau burden as assessed by florbetapir and [¹⁸F]-flortaucipir PET, respectively, and volumetric magnetic resonance imaging MRI (vMRI).

Patient population and study design: This study is a multi-center, randomized, double-blind, placebo-controlled study assessing the safety, adverse events, and efficacy of donanemab in participants with early symptomatic AD (the combination of prodromal AD, the symptomatic pre-dementia phase of AD where MCI is apparent [MCI-AD], and mild AD dementia [symptoms are sufficiently severe to meet dementia and AD diagnostic criteria]) aged 60-85 years (Dubois et al., “Research Criteria for the Diagnosis of Alzheimer's Disease: Revising the NINCDS-ADRDA Criteria,” The Lancet Neurology 6:734-46 (2007), which is hereby incorporated by reference in its entirety). Screening procedures included the Mini-Mental State Examination (MMSE; range 0 to 30, lower indicating greater impairment, Folstein et al., “Mini-mental state. A Practical Method for Grading the Cognitive State of Patients for the Clinician,” J. Psychiatr. Res. 12:189-98 (1975), which is hereby incorporated by reference in its entirety)), [¹⁸F]-flortaucipir PET scan, magnetic resonance imaging (MRI), and [¹⁸F]-florbetapir PET scan. The flortaucipir and [¹⁸F]-florbetapir PET scans were reviewed by a centralized PET imaging facility for assessment of patient's eligibility. All eligible patients were required to have evidence of pathologic tau on PET scan and quantitative tau levels below a specific upper threshold. The latter criterion addressed the concern that anti-amyloid treatments would have limited efficacy in advanced disease, as indicated by the presence of extensive tau pathology. Tau images were quantitatively evaluated to estimate an SUVr (standardized uptake value ratio) by published methods (Pontecorvo et al., “A Multicentre Longitudinal Study of Flortaucipir (18F) in Normal Ageing, Mild Cognitive Impairment and Alzheimer's Disease Dementia,” Brain 142:1723-35 (2019); Devous et al., “Test-Retest Reproducibility for the Tau PET Imaging Agent Flortaucipir F18,” Journal of Nuclear Medicine 59:937-43 (2018); Southekal et al., “Flortaucipir F18 Quantitation Using Parametric Estimation of Reference Signal Intensity,” J. Nucl. Med. 59:944-51 (2018), which are hereby incorporated by reference in their entireties) and visually evaluated (Fleisher et al., “Positron Emission Tomography Imaging With [¹⁸F]-flortaucipir and Postmortem Assessment of Alzheimer Disease Neuropathologic Changes,” JAMA Neurology 77:829-39 (2020), which is hereby incorporated by reference in its entirety) for whether they had an AD pattern.

Any image with an SUVr>1.46 was excluded as having high tau. For those images not excluded as having high tau, images with SUVr values<1.10 or images that were visually read as having a negative AD pattern were excluded for having inadequate tau levels with the exception that if the image was visually read as having an advanced tau AD pattern but SUVr value was <1.10, the case would still be included. Except for MRI, each patient was required to meet all other Visit 1 eligibility criteria prior to a screening [¹⁸F]-florbetapir PET scan.

Participants who met entry criteria were randomized 1:1 to receive either intravenous (IV) donanemab every 4 weeks (700 mg for the first 3 doses, 1400 mg thereafter) or IV placebo every 4 weeks, for up to 72 weeks. For between-group comparability for site factor, participant randomization was stratified by investigative site. There was no stratification by entry criteria. In participants treated with donanemab, the dose was downtitrated to 700 mg if amyloid removal in centiloids (CL) measured by florbetapir scan (24 and 52 weeks) was ≥11 and <25, or switched to placebo if <11 at any one measure or ≥11 and <25 for two consecutive scans. If amyloid-related imaging abnormalities-edema/effusions (ARIA-E; signal hyperintensities on an MRI in fluid-attenuated inversion recovery imaging sequences, due to parenchymal fluid accumulation or sulcal fluid effusion; Sperling et al., “Amyloid-related Imaging Abnormalities in Amyloid-Modifying Therapeutic Trials: Recommendations from the Alzheimer's Association Research Roundtable Workgroup,” Alzheimer's & Dementia 7:367-85 (2011), which is hereby incorporated by reference in its entirety) occurred during the up-titration with the first three doses of 700 mg, the dose was not escalated. Final endpoint measures and safety assessments were performed at Week 76, 4 weeks after the last infusion.

Clinical and biomarker outcome measures: The primary outcome measure was the change from baseline to 76 weeks in the change on the iADRS (range 0 to 144, lower indicating greater cognitive deficit and impairment of daily living; Wessels et al., “A Combined Measure of Cognition and Function for Clinical Trials: The Integrated Alzheimer's Disease Rating Scale (iADRS),” J. Prev. Alzheimer's Dis. 2:227-41 (2015), which is hereby incorporated by reference in its entirety), compared with placebo. The iADRS is a linear combination of its individual components, the AD Assessment Scale-Cognitive (ADAS-Cog₁₃; range 0 to 85, higher indicating greater disease severity; Mohs et al., “Development of Cognitive Instruments for use in Clinical Trials of Antidementia Drugs: Additions to the Alzheimer's Disease Assessment Scale that Broaden its Scope. The Alzheimer's Disease Cooperative Study,” Alzheimer Dis Assoc Disord 11 Suppl 2:S13-21 (1997), which is hereby incorporated by reference in its entirety) and the AD Cooperative Study-instrumental Activities of Daily Living (ADCS-iADL; range 0 to 59, lower indicating greater impairment; Galasko et al., “An Inventory to Assess Activities of Daily Living for Clinical Trials in Alzheimer's disease,” Alzheimer Disease and Associated Disorders 11:S33-S9 (1997) and Galasko et al., “Galantamine Maintains Ability to Perform Activities of Daily Living in Patients with Alzheimer's Disease,” Journal of the American Geriatrics Society 52:1070-6 (2004), which are hereby incorporated by reference in their entirety).

The iADRS was developed using the theoretical construct of aiming to measure core disease processes and clinical trial data was used to identify items/scales that performed best for that construct. All items of the ADAS-Cog₁₃ total score and ADCS-iADL score are included without weighting of items, yielding face validity and ease of interpretation of both the composite and its components. The iADRS allows for an overall measure of AD impairment (total score) as well as individual subscores (cognition and function). Validation of iADRS has been established and statistical properties of the composite performance have been described.

Methodologies for the secondary outcome measures, Clinical Dementia Rating Scale Sum of Boxes (CDR-SB; range 0 to 18, higher indicating greater impairment; Morris, “The Clinical Dementia Rating (CDR),” Current Version and Scoring Rules 43:2412—a (1993), which is hereby incorporated by reference in its entirety), ADAS-Cog₁₃, ADCS-iADL, MMSE, amyloid and tau burden as assessed by florbetapir and [¹⁸F]-flortaucipir PET respectively, and volumetric MRI are detailed in the protocol. Assessment of global tau load was carried out using a Tau^{IQ} algorithm (Whittington et al., “TauIQ-A Canonical Image Based Algorithm to Quantify Tau PET Scans,” J. of Nuclear Medicine (2021), which is hereby incorporated by reference in its entirety) accounting for the spatiotemporal distribution of tau.

Determination of sample size and statistical analysis: Enrolment of 250 participants randomized 1:1 to two treatment arms, with 200 participants expected to complete treatment, was determined to provide approximately 84% power to demonstrate that the active treatment arm had a ≥0.6 posterior probability of slowing iADRS progression over placebo by at least 25%. The assumption for power calculation was mean progression levels in the placebo and donanemab arms of approximately 12 and 6 points (50% slowing) over 18 months, respectively, with common standard deviation of 17. Efficacy analyses were conducted based on a modified intention-to-treat principle (unless otherwise specified) where participants had a baseline and at least one post-baseline iADRS measurement. Unless otherwise noted, all pairwise tests of treatment effects were conducted at a 2-sided alpha level of 0.05.

Baseline characteristics were summarized by treatment group and overall, with descriptive statistics for continuous and categorical measures. The primary outcome was analyzed with the use of a mixed-model repeated-measures (MMRM) analysis, with the change from baseline in the iADRS score at each scheduled postbaseline timepoint as the dependent variable. The model for the fixed effects included the following terms: baseline score, investigator, treatment, visit, treatment-by-visit interaction, baseline-by-visit interaction, concomitant acetylcholinesterase inhibitors and/or memantine use at baseline (yes/no), and age at baseline. Visit was considered a categorical variable. Secondary efficacy outcomes were assessed using a MMRM analysis. Bretz's graphical approach (Bretz, et. al., “A Graphical Approach to Sequentially Rejective Multiple Test Procedures,” Statistics in Medicine, 28(4):586-604 (2009), which is hereby incorporated by reference in its entirety) was used to provide control of the study-wise type I error rate for the primary and key secondary hypotheses at alpha level of 0.05. Assuming that the primary analysis was significant, the MMRM analyses described for the primary analysis was conducted on the CDR-SB, ADAS-Cogn, ADCS-iADL and MMSE scores and significance determined based on a multiplicity graph of hypotheses. Longitudinal clinical outcomes are provided with point estimates and error bars. For post-baseline categorical data, Fisher's exact test was used for treatment-group comparisons. For post-baseline continuous data collected at endpoint, analysis of covariance (ANCOVA), with independent factors for treatment and age, was used. Each principal site investigator was responsible for selecting raters, who met training requirements, to administer the instruments at the site. Raters were blinded to treatment assignment.

The Bayesian Disease Progression Model (DPM) was used to assess the rate of decline of the iADRS between the donanemab group and the placebo group across the 76 weeks of the study, as pre-specified in the protocol. The model assumes a proportional treatment effect relative to placebo and includes diffuse priors. A similar model was previously used, with the exception that in the current model the prior distributions on the parameters representing the placebo decline were not forced to be monotonic. The analysis generated a posterior probability distribution of the disease progression ratio (DPR), defined as the proportional decline of the donanemab arm relative to placebo. A DPR of less than 1 favors donanemab. The 95% credible intervals and the posterior mean of the disease progression ratios are presented. The posterior probability of the active treatment arm slowing the disease progression by at least 25% relative to placebo was pre-specified and calculated from the DPM. The DPM model was used to assess the rate of decline of the CDR-SB, ADAS-Cog₁₃, ADCS-iADL and MMSE. DPM models were not included as part of our pre-specified multiplicity testing strategy for secondary endpoints.

Safety parameters (AEs, laboratory analytes, vital signs, electrocardiograms, and MRIs) were summarized using descriptive statistics for continuous variables and frequencies along with percentages for categorical variables during the treatment period.

A likelihood-based mixed effects model for repeated measures was used to handle missing data for the MMRM model. The model parameters were simultaneously estimated using restricted likelihood estimation incorporating all observed data. Estimates have been shown to be unbiased when the missing data are missing at random and when there is ignorable non-random missing data. Repeated measures analyses only used data from visits where the data was scheduled to be collected. When participants discontinued from the study early, there may have been efficacy or safety data measurements at visits where the variables were not scheduled to be collected. This data was used in all other analyses.

Population and Baseline Characteristics: Baseline demographics for the placebo and donanemab monotherapy groups, respectively, for mean age were 75.4 and 75.0 years, for female sex were 51.6% and 51.9%, for white race were 96.0% and 93.1%, and for APOE4 carrier were 74.2% and 72.5% (Table B).

TABLE B
Characteristics of Trial Participants at Baseline

| | | | Donanemab | | | |
|---|---|---|---|---|---|---|
| | Placebo | | Monotherapy | | Total † | |
| | (N = 126) | | (N = 131) | | (N = 272) | |
| Demographic | | | | | | |
| Female sex, n (%) | 65 | (51.6) | 68 | (51.9) | 145 | (53.3) |
| Mean age, years (SD) | 75.4 | (5.4) | 75.0 | (5.6) | 75.2 | (5.5) |
| Race, n (%) | | | | | | |
| Asian | 2 | (1.6) | 1 | (0.8) | 3 | (1.1) |
| Black or African American | 3 | (2.4) | 5 | (3.8) | 8 | (2.9) |
| White | 121 | (96.0) | 122 | (93.1) | 258 | (94.9) |
| Other* | 0 | (0) | 3 | (2.3) | 3 | (1.1) |
| Ethnicity, Hispanic/Latino ^{#}, n (%) | 3 | (2.4) | 5 | (3.8) | 9 | (3.3) |
| Education, ≥13 years, n (%) | 102 | (81.0) | 97 | (74.0) | 209 | (76.8) |
| APOE 4 carrier, n/N (%) | 92/124 | (74.2) | 95/131 | (72.5) | 197/270 | (73.0) |
| E2/E3, n (%) | 1 | (0.8) | 1 | (0.8) | 2 | (0.7) |
| E2/E4, n (%) | 2 | (1.6) | 2 | (1.5) | 4 | (1.5) |
| E3/E3, n (%) | 31 | (25.0) | 35 | (26.7) | 71 | (26.3) |
| E3/E4, n (%) | 62 | (50.0) | 68 | (51.9) | 137 | (50.7) |
| E4/E4, n (%) | 28 | (22.6) | 25 | (19.1) | 56 | (20.7) |
| AChEI use, n (%) | 74 | (58.7) | 78 | (59.5) | 162 | (59.6) |

| | | | |
|---|---|---|---|
| Scale, Mean (SD), range | | | |
| iADRS | 105.9 (13.2), | 106.2 (13.0) ^{a}, | 106.2 (13.0) ^{b}, |
| | 67.0-139.0 | 60.0-130.0 | 60.0-139.0 |
| CDR-SB | 3.4 (1.7), | 3.6 (2.1), | 3.5 (1.9), |
| | 0.5-8.0 | 0.5-11.0 | 0.5-11.0 |
| ADAS-Cog₁₃ | 27.5 (7.6), | 27.6 (7.7), | 27.6 (7.6), |
| | 5.0-47.0 | 10.0-51.0 | 5.0-51.0 |
| ADCS-ADL | 67.0 (8.1), | 67.4 (8.6) ^{a}, | 67.3 (8.2) ^{b}, |
| | 40.0-78.0 | 28.0-78.0 | 28.0-78.0 |
| ADCS-iADL | 48.4 (7.5), | 48.9 (7.6) ^{a}, | 48.8 (7.5) ^{b}, |
| | 24.0-59.0 | 21.0-59.0 | 21.0-59.0 |
| MMSE | 23.7 (2.9) ^{c}, | 23.6 (3.1) ^{d}, | 23.5 (3.1) ^{e}, |
| | 16.0-29.0 | 14.0-29.0 | 13.0-30.0 |
| Amyloid PET Centiloids, | 101.1 (33.3), | 107.6 (36.0), | 104.2 (34.8), |
| Mean (SD), range | 38.7-225.2 | 41.0-251.4 | 38.7-251.4 |
| Flortaucipir PET global tau | 0.46 (0.15) ^{f}, | 0.47 (0.19) ^{g}, | 0.46 (0.17) ^{h}, |
| load, Mean (SD), range | 0.2-0.9 | 0.1-1.2 | 0.1-1.2 |

*Note:
includes Multiple & American Indian or Alaska Native.
† includes participants in the combo group.
^{#} number of participants with non-missing data, used as denominator,
^{a} Donanemab monotherapy N = 130,
^{b} Total N = 271,
^{c} Placebo N = 121,
^{d} Donanemab monotherapy N = 126,
^{e} Total N = 261,
^{f} Placebo N = 124,
^{g} Donanemab monotherapy N = 130,
^{h} Total N = 269.
APOE 4 = Apolipoprotein E allele 4; AChEI = Acetylcholinesterase Inhibitor; ADAS-Cog₁₃ = AD Assessment Scale-Cognitive 13-item Subscale; ADCS-ADL = Alzheimer's Disease Cooperative Study Activities of Daily Living Inventory; ADCS-iADL = Alzheimer's Disease Cooperative Study-instrumental Activities of Daily Living Inventory; iADRS = integrated Alzheimer's Disease Rating Scale; MMSE = Mini-Mental State Examination; CDR-SB = Clinical Dementia Rating Scale Sum of Boxes; PET = Positron Emission Tomography; N/n = Number of participants; SD = Standard Deviation.

At the time of trial initiation, the study consisted of three arms including a combination group of donanemab with BACE 1 inhibitor. This arm was discontinued early in the trial with 15 participants randomized to that group. In the modified intention-to-treat population, of the 1955 participants screened, 126 were randomized to placebo, 131 to donanemab. The mean baseline scores for iADRS were 105.9 for placebo and 106.2 for donanemab; respectively for MMSE were 23.7 and 23.6; CDR-SB were 3.4 and 3.6; [¹⁸F]-flortaucipir PET global tau loads were 0.46 and 0.47; amyloid PET values were 101.1 and 107.6 (Table B).

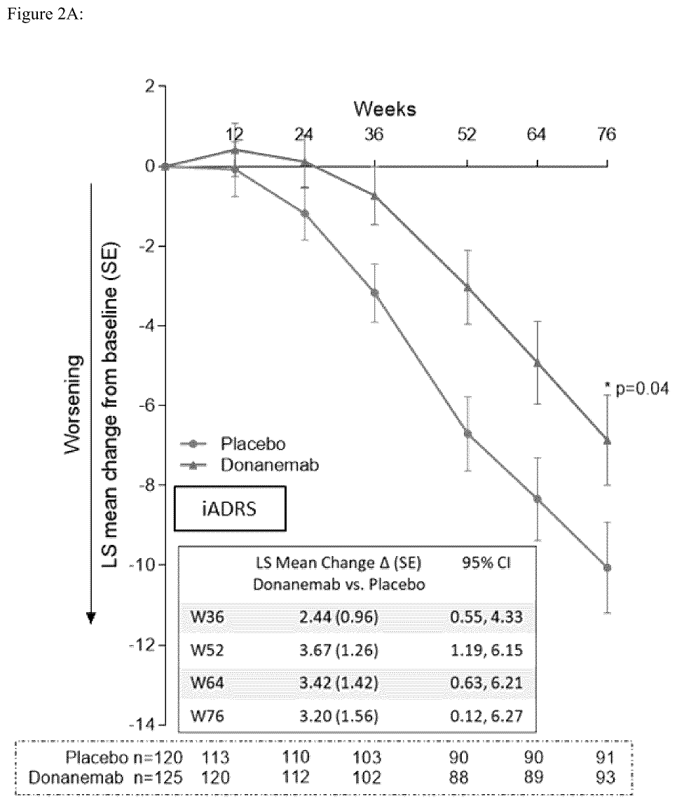

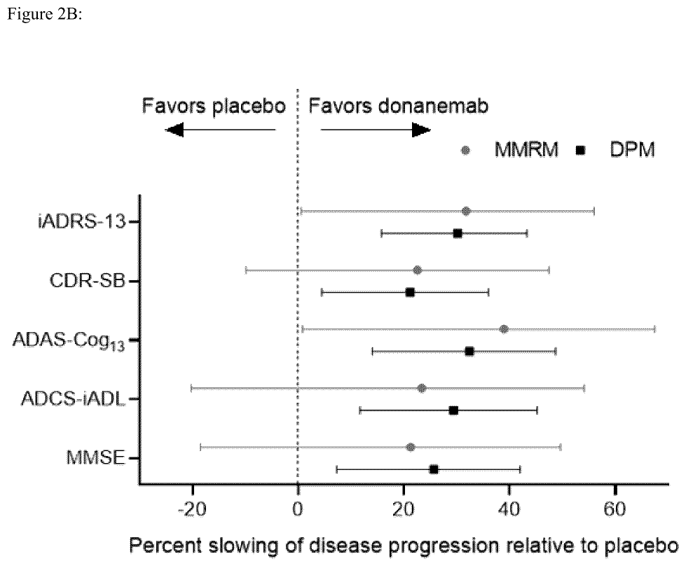

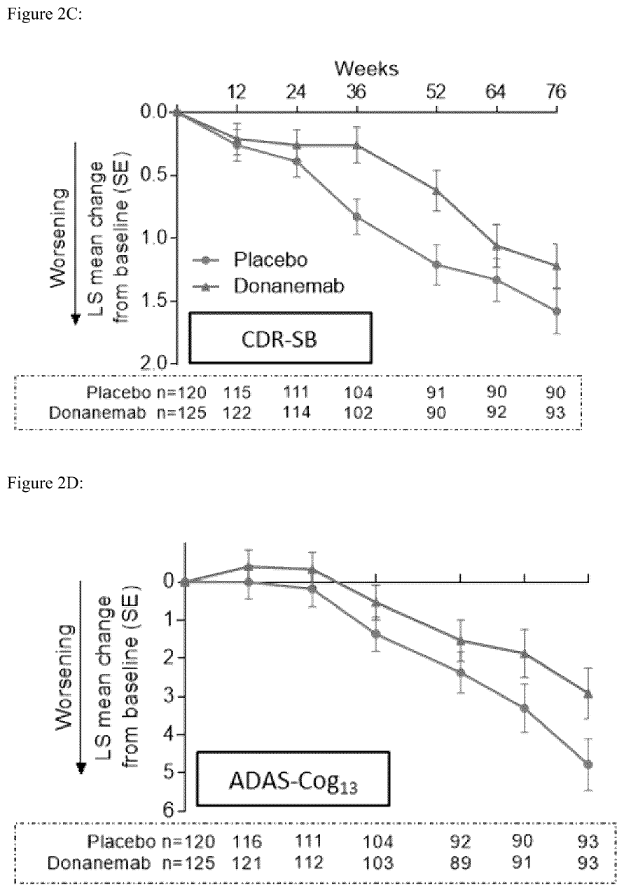

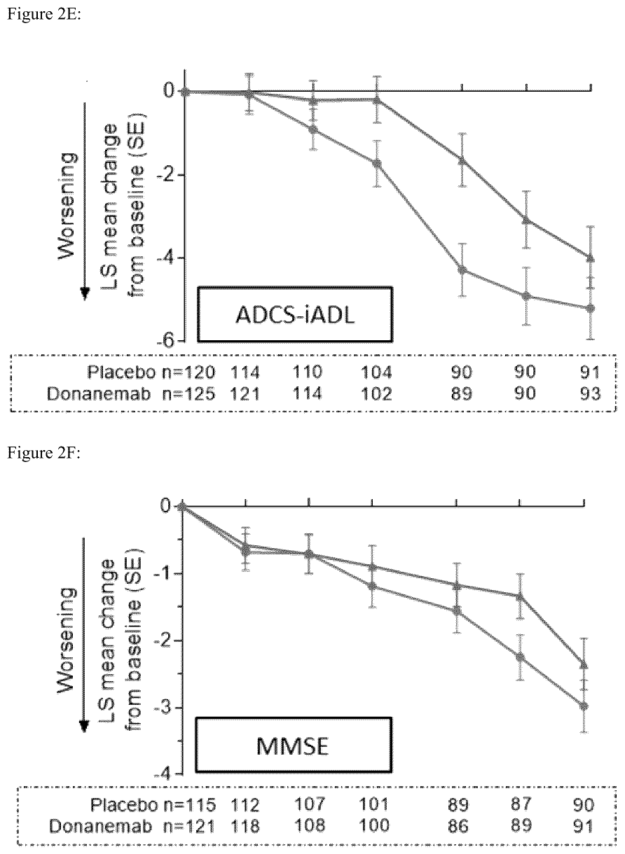

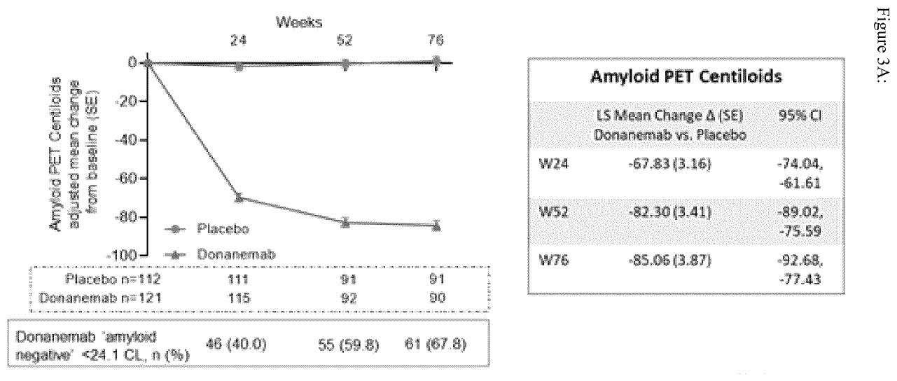

Primary Outcome: Donanemab showed significant slowing of decline in a composite measure of cognition and daily function in patients with early symptomatic Alzheimer's disease compared to placebo. Donanemab met the primary endpoint of change from baseline to 76 weeks in the Integrated Alzheimer's Disease Rating Scale (iADRS), slowing decline by 32% relative to placebo (FIG. 2A), which was statistically significant. The iADRS is a clinical composite tool combining the cognitive measure ADAS-Cog₁₃ and functional measure ADCS-iADL, two commonly used measures in Alzheimer's disease. The change from baseline on iADRS at 76 weeks was −10.06 for placebo and −6.86 for donanemab treated patients (treatment difference: 3.20, 95% confidence interval [CI]: 0.12, 6.27; p=0.04) (FIG. 2A and Table C).

FIGS. 2A-F illustrate clinical outcomes for primary iADRS, and secondary CDR-SB, ADAS-Cog₁₃, ADCS-iADL, and MMSE. FIG. 2A shows the results for the primary outcome, the LS mean change from baseline to 76 weeks in iADRS score, analyzed with MMRM. FIG. 2B shows the percent slowing estimates from the MMRM model at the 18-month endpoint, and the Bayesian DPM model over the entire 18-month study. 95% credible intervals are shown. FIGS. 2C-F show the results for secondary outcomes, the LS mean change from baseline to 76 weeks in CDR-SB (FIG. 2C), ADAS-Cog₁₃ (FIG. 2D), ADCS-iADL (FIG. 2E), and MMSE scores (FIG. 2F), analyzed with MMRM. In FIGS. 2A-F, A=Difference; W=Week; iADRS=integrated Alzheimer's Disease Rating Scale; ADAS-Cog₁₃=Alzheimer's Disease Assessment Scale-Cognitive subscale; ADCS-iADL=Alzheimer's Disease Cooperative Study-instrumental Activities of Daily Living scale; CDR-SB=Clinical Dementia Rating Scale Sum of Boxes; MMSE=Mini Mental State Examination; MMRM=Mixed Models for Repeated Measures; DPM=Disease Progression Model; LS=Least Squares; CI=Confidence Interval; n=Number of participants; SE=Standard Error.

TABLE C
Primary iADRS, and Secondary CDR-SB,
ADAS-Cog₁₃, ADCS-iADL, and MMSE Clinical Outcomes

| | | | | |
|---|---|---|---|---|
| iADRS | CDR-SB | ADAS-Cog₁₃ | ADCS-iADL | MMSE |

Week 12

| | | | | | | | |
|---|---|---|---|---|---|---|---|
| Placebo, LS mean | −0.07 | (0.684) | 0.26 (0.126) | −0.00 | (0.438) | −0.08 (0.455) | −0.68 (0.272) |
| change (SE) | | | | | | | |
| Donanemab, LS | 0.42 | (0.673) | 0.21 (0.124) | −0.40 | (0.433) | −0.02 (0.448) | −0.58 (0.267) |
| mean change (SE) | | | | | | | |

| | | | | | |
|---|---|---|---|---|---|
| LS mean change | 0.49 | −0.05 | −0.40 | 0.06 | 0.10 |
| difference (95% CI) | (−1.24, 2.22) | (−0.37, 0.27) | (−1.51, 0.71) | (−1.07, 1.20) | (−0.58, 0.78) |

Week 24

| | | | | | | | |
|---|---|---|---|---|---|---|---|
| Placebo, LS mean | −1.18 | (0.666) | 0.39 (0.123) | 0.18 | (0.451) | −0.91 (0.481) | −0.70 (0.295) |
| change (SE) | | | | | | | |
| Donanemab, LS | 0.12 | (0.663) | 0.26 (0.123) | −0.33 | (0.450) | −0.21 (0.478) | −0.71 (0.292) |
| mean change (SE) | | | | | | | |

| | | | | | |
|---|---|---|---|---|---|
| LS mean change | 1.30 | −0.13 | −0.51 | 0.69 | −0.01 |
| difference (95% CI) | (−0.38, 2.99) | (−0.44, 0.18) | (−1.66, 0.65) | (−0.52, 1.91) | (−0.77, 0.74) |

Week 36

| | | | | | | | |
|---|---|---|---|---|---|---|---|
| Placebo, LS mean | −3.17 | (0.730) | 0.83 (0.139) | 1.36 | (0.445) | −1.73 (0.556) | −1.19 (0.314) |
| change (SE) | | | | | | | |
| Donanemab, LS | −0.73 | (0.732) | 0.26 (0.140) | 0.53 | (0.446) | −0.19 (0.558) | −0.89 (0.312) |
| mean change (SE) | | | | | | | |

| | | | | | |
|---|---|---|---|---|---|
| LS mean change | 2.44 | −0.56 | −0.84 | 1.54 | 0.30 |
| difference (95% CI) | (0.55, 4.33) | (−0.92, −0.20) | (−1.98, 0.30) | (0.09, 2.99) | (−0.51, 1.11) |

Week 52

| | | | | | | | |
|---|---|---|---|---|---|---|---|
| Placebo, LS mean | −6.70 | (0.929) | 1.21 (0.160) | 2.37 | (0.536) | −4.28 (0.635) | −1.56 (0.321) |
| change (SE) | | | | | | | |
| Donanemab, LS | −3.03 | (0.933) | 0.62 (0.160) | 1.53 | (0.540) | −1.64 (0.637) | −1.17 (0.321) |
| mean change (SE) | | | | | | | |

| | | | | | |
|---|---|---|---|---|---|
| LS mean change | 3.67 | −0.59 | −0.84 | 2.64 | 0.40 |
| difference (95% CI) | (1.19, 6.15) | (−1.01, −0.16) | (−2.25, 0.58) | (0.96, 4.33) | (−0.44, 1.23) |

Week 64

| | | | | | | | |
|---|---|---|---|---|---|---|---|
| Placebo, LS mean | −8.34 | (1.038) | 1.33 (0.171) | 3.30 | (0.621) | −4.91 (0.689) | −2.25 (0.339) |
| change (SE) | | | | | | | |
| Donanemab, LS | −4.92 | (1.038) | 1.06 (0.170) | 1.87 | (0.619) | −3.07 (0.687) | −1.34 (0.335) |
| mean change (SE) | | | | | | | |

| | | | | | |
|---|---|---|---|---|---|
| LS mean change | 3.42 | −0.27 | −1.43 | 1.85 | 0.91 |
| difference (95% CI) | (0.63, 6.21) | (−0.72, 0.18) | (−3.09, 0.23) | (0.01, 3.69) | (0.02, 1.79) |

Week 76

| | | | | | | | |
|---|---|---|---|---|---|---|---|
| Placebo, LS mean | −10.06 | (1.141) | 1.58 (0.178) | 4.77 | (0.660) | −5.20 (0.743) | −2.98 (0.390) |
| change (SE) | | | | | | | |
| Donanemab, LS | −6.86 | (1.135) | 1.22 (0.176) | 2.91 | (0.659) | −3.98 (0.738) | −2.35 (0.386) |
| mean change (SE) | | | | | | | |

| | | | | | |
|---|---|---|---|---|---|
| LS mean change | 3.20 | −0.36 | −1.86 | 1.21 | 0.64 |
| difference (95% CI) | (0.12, 6.27) | (−0.83, 0.12) | (−3.63, −0.09) | (−0.77, 3.20) | (−0.40, 1.67) |

Results for the mean change from baseline for the primary iADRS, and secondary ADAS-Cog₁₃, ADCS-iADL, CDR-SB, and MMSE clinical outcomes, analyzed with MMRM. iADRS = integrated Alzheimer's Disease Rating Scale; ADAS-Cog₁₃ = Alzheimer's Disease Assessment Scale-Cognitive subscale; ADCS-iADL = Alzheimer's Disease Cooperative Study-instrumental Activities of Daily Living scale; CDR-SB = Clinical Dementia Rating Scale Sum of Boxes; MMSE = Mini Mental State Examination; MMRM = Mixed Models for Repeated Measures; LS = Least Squares; CI = Confidence Interval; SE = Standard Error

Percent slowing estimates of disease progression relative to placebo, from the MMRM model at the 18-month endpoint and the Bayesian DPM over the entire 18-months, showed slowing of decline in the iADRS with both methods (FIG. 2B). The posterior probability of at least 25% slowing of disease progression relative to placebo on the iADRS was calculated as 0.78 from the Bayesian DPM.

Secondary Outcome: Donanemab also showed consistent improvements in all prespecified secondary endpoints measuring cognition and function compared to placebo but did not reach nominal statistical significance on every secondary endpoint. In the donanemab group, the observed difference from placebo in change from baseline at 76 weeks for CDR-SB was −0.36 (95% CI: −0.83 to 0.12), for ADAS-Cog₁₃ was −1.86 (95% CI: −3.63 to −0.09), for ADCS-iADL was 1.21 (95% CI: −0.77 to 3.20), and for MMSE was 0.64 (95% CI: −0.40 to 1.67) (FIGS. 2C-F and Table D).

TABLE D
Primary iADRS, and secondary CDR-SB, ADAS-Cog₁₃, ADCS-iADL, and MMSE clinical outcomes

| | | | | |
|---|---|---|---|---|
| iADRS | CDR-SB | ADAS-Cog₁₃ | ADCS-iADL | MMSE |

Week 12

| | | | | | | | |
|---|---|---|---|---|---|---|---|
| Placebo, LS mean | −0.07 | (0.684) | 0.26 (0.126) | −0.00 | (0.438) | −0.08 (0.455) | −0.68 (0.272) |
| change (SE) | | | | | | | |
| Donanemab, LS | 0.42 | (0.673) | 0.21 (0.124) | −0.40 | (0.433) | −0.02 (0.448) | −0.58 (0.267) |
| mean change (SE) | | | | | | | |

| | | | | | |
|---|---|---|---|---|---|
| LS mean change | 0.49 | −0.05 | −0.40 | 0.06 | 0.10 |
| difference (95% CI) | (−1.24, 2.22) | (−0.37, 0.27) | (−1.51, 0.71) | (−1.07, 1.20) | (−0.58, 0.78) |

Week 24

| | | | | | | | |
|---|---|---|---|---|---|---|---|
| Placebo, LS mean | −1.18 | (0.666) | 0.39 (0.123) | 0.18 | (0.451) | −0.91 (0.481) | −0.70 (0.295) |
| change (SE) | | | | | | | |
| Donanemab, LS | 0.12 | (0.663) | 0.26 (0.123) | −0.33 | (0.450) | −0.21 (0.478) | −0.71 (0.292) |
| mean change (SE) | | | | | | | |

| | | | | | |
|---|---|---|---|---|---|
| LS mean change | 1.30 | −0.13 | −0.51 | 0.69 | −0.01 |
| difference (95% CI) | (−0.38, 2.99) | (−0.44, 0.18) | (−1.66, 0.65) | (−0.52, 1.91) | (−0.77, 0.74) |

Week 36

| | | | | | | | |
|---|---|---|---|---|---|---|---|
| Placebo, LS mean | −3.17 | (0.730) | 0.83 (0.139) | 1.36 | (0.445) | −1.73 (0.556) | −1.19 (0.314) |
| change (SE) | | | | | | | |
| Donanemab, LS | −0.73 | (0.732) | 0.26 (0.140) | 0.53 | (0.446) | −0.19 (0.558) | −0.89 (0.312) |
| mean change (SE) | | | | | | | |

| | | | | | |
|---|---|---|---|---|---|
| LS mean change | 2.44 | −0.56 | −0.84 | 1.54 | 0.30 |
| difference (95% CI) | (0.55, 4.33) | (−0.92, −0.20) | (−1.98, 0.30) | (0.09, 2.99) | (−0.51, 1.11) |

Week 52

| | | | | | | | |
|---|---|---|---|---|---|---|---|
| Placebo, LS mean | −6.70 | (0.929) | 1.21 (0.160) | 2.37 | (0.536) | −4.28 (0.635) | −1.56 (0.321) |
| change (SE) | | | | | | | |
| Donanemab, LS | −3.03 | (0.933) | 0.62 (0.160) | 1.53 | (0.540) | −1.64 (0.637) | −1.17 (0.321) |
| mean change (SE) | | | | | | | |

| | | | | | |
|---|---|---|---|---|---|
| LS mean change | 3.67 | −0.59 | −0.84 | 2.64 | 0.40 |
| difference (95% CI) | (1.19, 6.15) | (−1.01, −0.16) | (−2.25, 0.58) | (0.96, 4.33) | (−0.44, 1.23) |

Week 64

| | | | | | | | |
|---|---|---|---|---|---|---|---|
| Placebo, LS mean | −8.34 | (1.038) | 1.33 (0.171) | 3.30 | (0.621) | −4.91 (0.689) | −2.25 (0.339) |
| change (SE) | | | | | | | |
| Donanemab, LS | −4.92 | (1.038) | 1.06 (0.170) | 1.87 | (0.619) | −3.07 (0.687) | −1.34 (0.335) |
| mean change (SE) | | | | | | | |

| | | | | | |
|---|---|---|---|---|---|
| LS mean change | 3.42 | −0.27 | −1.43 | 1.85 | 0.91 |
| difference (95% CI) | (0.63, 6.21) | (−0.72, 0.18) | (−3.09, 0.23) | (0.01, 3.69) | (0.02, 1.79) |

Week 76

| | | | | | | | |
|---|---|---|---|---|---|---|---|
| Placebo, LS mean | −10.06 | (1.141) | 1.58 (0.178) | 4.77 | (0.660) | −5.20 (0.743) | −2.98 (0.390) |
| change (SE) | | | | | | | |
| Donanemab, LS | −6.86 | (1.135) | 1.22 (0.176) | 2.91 | (0.659) | −3.98 (0.738) | −2.35 (0.386) |
| mean change (SE) | | | | | | | |

| | | | | | |
|---|---|---|---|---|---|
| LS mean change | 3.20 | −0.36 | −1.86 | 1.21 | 0.64 |
| difference (95% CI) | (0.12, 6.27) | (−0.83, 0.12) | (−3.63, −0.09) | (−0.77, 3.20) | (−0.40, 1.67) |

Results for the mean change from baseline for the primary iADRS, and secondary ADAS-Cog₁₃, ADCS-iADL, CDR-SB, and MMSE clinical outcomes, analyzed with MMRM. iADRS = integrated Alzheimer's Disease Rating Scale; ADAS-Cog₁₃ = Alzheimer's Disease Assessment Scale-Cognitive subscale; ADCS-iADL = Alzheimer's Disease Cooperative Study-instrumental Activities of Daily Living scale; CDR-SB = Clinical Dementia Rating Scale Sum of Boxes; MMSE = Mini Mental State Examination; MMRM = Mixed Models for Repeated Measures; LS = Least Squares; CI = Confidence Interval; SE = Standard Error

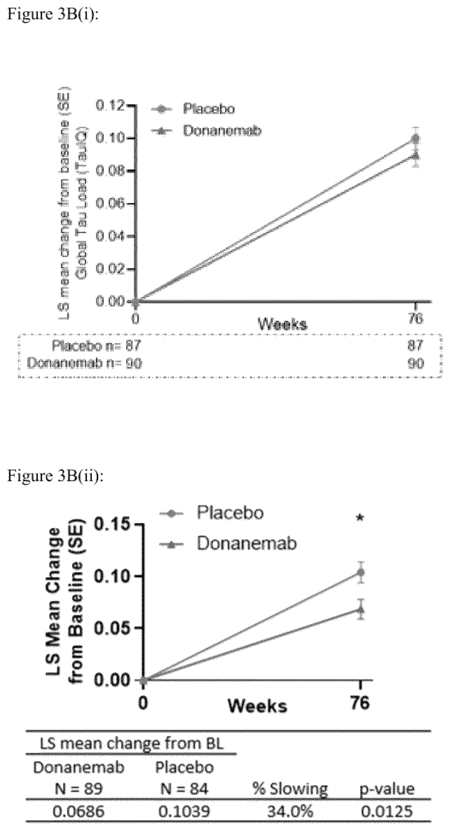

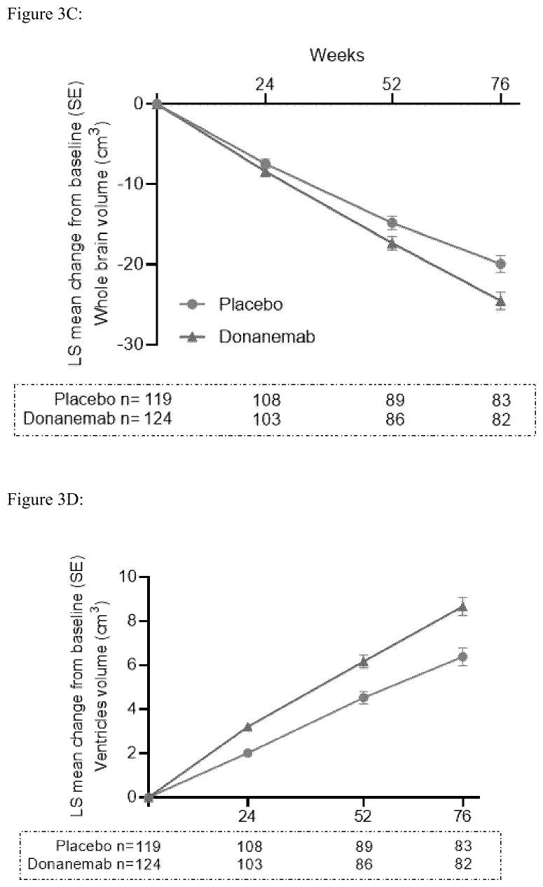

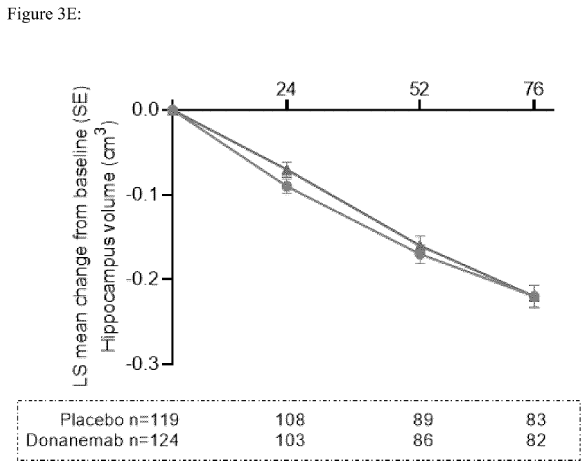

Biomarkers: By targeting N3pGlu Aβ, donanemab treatment has been shown to rapidly result in high levels of amyloid plaque clearance, as measured by amyloid imaging. For PET amyloid, participants treated with donanemab showed an 85 CL amyloid plaque reduction at 76 weeks, compared with placebo (placebo=0.93, donanemab=−84.13) (FIG. 3A). A separation of 68 CL reduction was evident in the donanemab group by 24 weeks, compared with placebo (placebo=−1.82, donanemab=−69.64; 65% reduction from baseline in donanemab group). The percentage of participants that were ‘amyloid negative’, as defined by <24.1 CL amyloid plaque, in the donanemab group, at 24, 52, and 76 weeks was 40.0%, 59.8%, and 67.8%, respectively (FIG. 3A). Approximately 27% and 55% of donanemab participants dosed at week 28 and week 56, respectively, achieved adequate amyloid lowering to reduce to placebo infusion. In this study, patients stopped receiving donanemab and switched to placebo once their amyloid plaque level was below 25 centiloids for two consecutive measures or below 11 centiloids at any one measure.

Evaluation of global tau load assessed by [¹⁸F]-flortaucipir PET revealed no difference between groups from baseline to 76 weeks (FIG. 3B(i)). FIG. 3B(ii), however, shows that there is a significant slowing of tau for the global measure MUBADA/cerebellum crus ref region. The figure illustrates the effect of donanemab on total tau (neurofibrillary tangles as measured by flortaucipir PET) progression throughout the entire brain, unlike the other analyses that focus on the individual lobes or regions. The MUBADA region represent a global region across the entire brain that corresponds to typical regions with neurofibrillary tangle accumulation consistent with Alzheimer's Disease. Treatment with donanemab has a statistically significant effect on slowing the progression of neurofibrillary tangles throughout the brain. In FIG. 3B(ii), “*” indicates p<0.05, BL=baseline; LS=least squares; MUBADA=multiblock barycentric discriminant analysis; N=number of participants; SE=standard error; SUVr=standardized uptake value ratio.

Hippocampal volume change, assessed with vMRI, showed no difference between groups (FIG. 3E). There was a greater whole brain volume decrease and greater ventricular volume increase in participants treated with donanemab at 52, compared with placebo (FIGS. 3C & D).

FIGS. 3A-E show the outcomes for secondary biomarkers. FIG. 3A shows results for the secondary outcomes, change from baseline to 76 weeks, in brain amyloid plaque deposition, as measured by [¹⁸F]-florbetapir PET scan in centiloids (CL). ‘Amyloid negative’/<24.1 CL=the average CL level for similar aged otherwise healthy individuals. FIG. 3B shows the global tau load, as measured by [¹⁸F]-flortaucipir PET scan. FIGS. 3C-E shows vMRI for whole brain (FIG. 3C), ventricles (FIG. 3D), and hippocampus (FIG. 3E). In FIGS. 3A-E, A=Difference; W=Week; LS=Least squares; CI=Confidence Interval; CL=Centiloids; n=Number of participants; SE=Standard Error.

Adverse Events: There was no difference in the incidence of death or serious adverse events (SAEs) between donanemab and placebo groups. A total of 113 of 125 participants (90.4%) in the placebo group and 119 of 131 (90.8%) in the donanemab group had at least one treatment-emergent adverse event (TEAE) during the double-blind period in the safety population. The incidence of ARIA-E was significantly higher in the donanemab group (27%), compared with placebo (0.8%). Symptomatic ARIA-E was reported by 6.1% of all participants in the donanemab group (22% of participants with ARIA-E), compared with 0.8% in the placebo group. Most ARIA-E cases occurred by week 12 of dosing initiation. Serious symptomatic ARIA-E requiring hospitalization occurred in 2 participants treated with donanemab (1.5%). Both participants had symptoms of confusion and one reported difficulty expressing themselves, all of which fully resolved. ARIA-E fully resolved in both cases, with a mean ARIA-E resolution time of 18 weeks. The incidence of superficial siderosis (a type of ARIA with hemorrhage (ARIA-H)) of the central nervous system, nausea, and infusion-related reactions (IRR) were all significantly higher in the donanemab group, compared with placebo. Treatment discontinuation due to ARIA-E occurred with 7 participants (5.3%) in the donanemab group; 2 (1.5%) discontinued the study due to ARIA-E. No brain macrohemorrhages were seen in either group. IRR were reported in 7.6% of participants on donanemab and 0% on placebo. Serious IRR or hypersensitivity occurred in 3 participants (2.3%) treated with donanemab. The incidence of treatment-emergent anti-drug antibodies (TE-ADAs) in participants treated with donanemab was approximately 90%.

These results show that in an amyloid-plaque-specific intervention in patients with early symptomatic AD, amyloid clearance in the donanemab arm was accompanied by a slowing of disease progression, compared with placebo. This 3.20 treatment difference at 76 weeks on the iADRS scale should be interpreted in the context of not only the score range across the entire disease spectrum (0 to 144) but also, importantly, the dynamic range of iADRS within the participant population (26 points) and the decline in the placebo group (−10.06).

The results provided here are unexpected and surprising in several aspects. The dosing regimen of donanemab provides high amounts of amyloid removal early in the trial with almost 60% of participants having an ‘amyloid negative’ scan by 52 weeks. This is the first study to screen all participants with [¹⁸F]-flortaucipir PET scans, likely narrowing the range of underlying pathology, which in turn, likely decreased variance of the clinical decline.

The tau PET screening of the patients excluded subjects with high tau. Patients with high tau are less responsive to anti-amyloid treatment or have a disease that is more resistant to anti-amyloid treatments.

As proposed by the European Prevention of Alzheimer's Dementia project, analysis of treatment differences for the iADRS, ADAS-Cog₁₃, ADCS-iADL, CDR-SB, and MMSE scores, was performed using a relatively novel disease progression model. Given better sensitivity for detecting treatment effects (Solomon et al., “European Prevention of Alzheimer's Dementia Longitudinal Cohort Study (EPAD LCS): Study Protocol,” BMJ Open 8:e021017 (2018), which is hereby incorporated by reference in its entirety), this model can allow for substantial gains in statistical power (Wang et al., “A Novel Cognitive Disease Progression Model for Clinical Trials in Autosomal-dominant Alzheimer's disease,” Statistics in Medicine 37:3047-55 (2018), which is hereby incorporated by reference in its entirety) and in this trial revealed similar estimates of disease slowing to the single point-estimate of the MRM model.

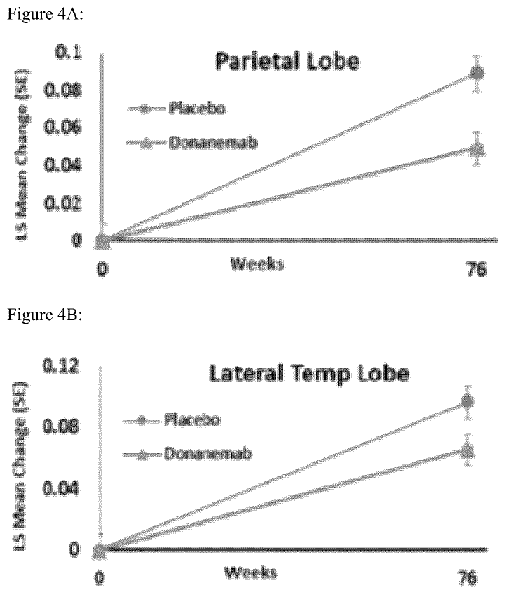

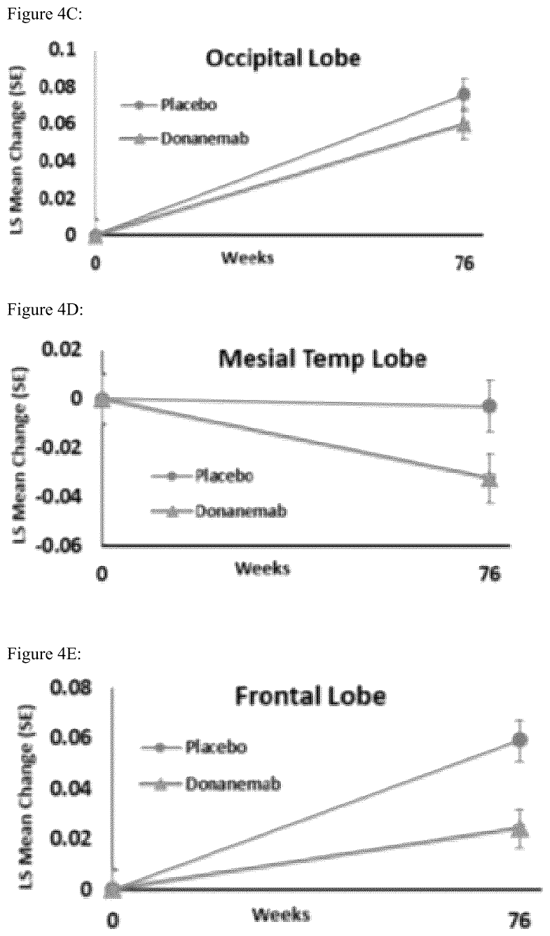

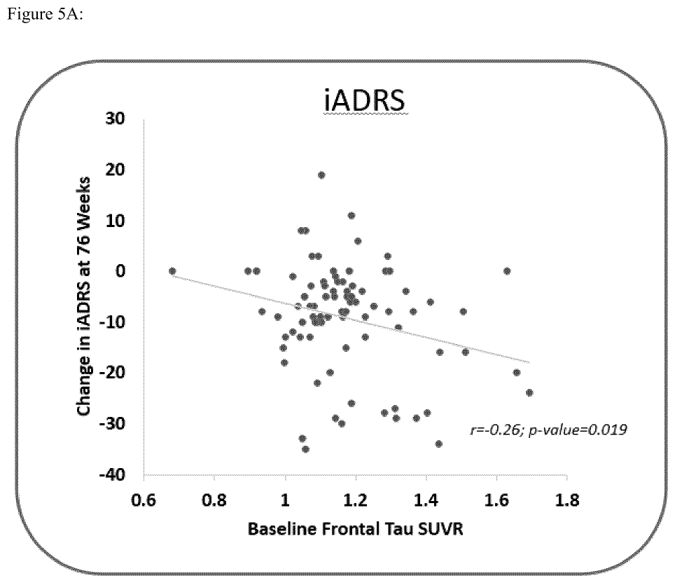

Regarding the observed lack of treatment effect on global tau load, it is conceivable that tau changes by PET will lag substantially relative to amyloid changes and that an 18-month time is too short to detect imaging changes. Modelling in autosomal dominant subjects suggest a lag of 10-20 years from first detectable PET amyloid changes and first detectable tau PET changes (Barthelemy et al., “A Soluble Phosphorylated Tau Signature Links Tau, Amyloid and the Evolution of Stages of Dominantly Inherited Alzheimer's Disease,” Nat. Med. 26:398-407 (2020), which is hereby incorporated by reference in its entirety). The lack of impact on global tau may prompt questions about whether targeting amyloid-β reduction affects biological disease progression. However, additional pre-specified analyses of brain regions suggest a reduction in tau accumulation in frontal and temporal lobe regions, in the donanemab group compared with placebo (FIGS. 4A-E).

A robust decrease or prevention of further increase of tau accumulation is seen in, e.g., the frontal lobes of the brain. It is important to note that statistical significance was not seen in the occipital lobe of the brain. This lobe has some of the highest baseline signal and may therefore provide a ceiling effect on the ability to show a decrease in the increasing tau load.

FIGS. 4A-E show regional SUVr analyses of tau accumulation with cerebellar gray reference. The frontal lobe tau load, as measured by flortaucipir, using a cerebellar reference region is correlated with the iADRS and CDR-SB change over the following 76 weeks in symptomatic early AD subjects. In FIGS. 4A-E, LS=Least Squares; SE=Standard Error; AAL Regions using posterior cerebellum gray matter reference regions. The frontal lobe shows a slowing in tau accumulation of 59.1% (P-Value: 0.0020); parietal lobe shows a slowing in tau accumulation of 44.6% (P-value: 0.0024); occipital lobe shows a slowing in tau accumulation of 21.0% (P-value: 0.2036); and lateral temporal lobe shows a slowing in tau accumulation of 31.8% (P-value: 0.0328).

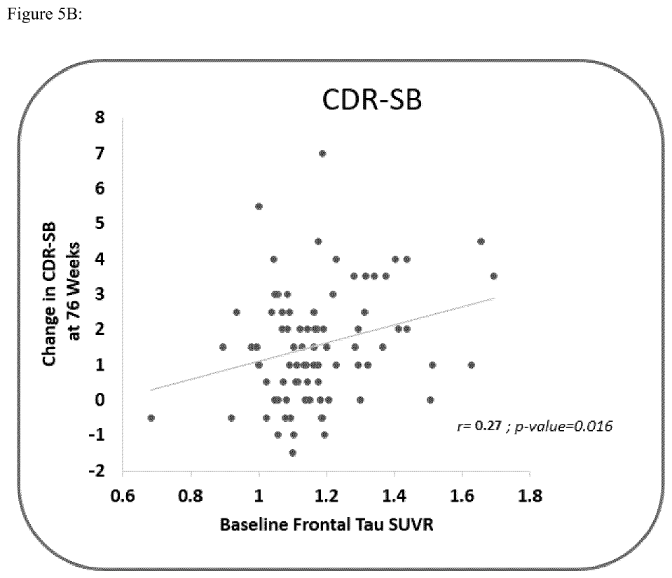

FIGS. 5A-B show the change in placebo group at 76 weeks vs. baseline frontal tau SUVR and that lower frontal tau burden is associated with less decline in the patients. High frontal lobe tau burden is associated with a fast decline in the patients. In other words, patients with low frontal lobe tau burden experience slower decline (as measured by iADRS or CDR-SB) as compared to patients with high frontal lobe tau burden.

This measurement reflects global changes in tau load and further exploration may show subregions that could be more sensitive to change. Optimal methods for region selection and analysis for quantifying tau changes and response to therapy remain in their infancy.

There was no significant change in hippocampal volume in contrast with the recent BACE inhibitor studies that showed significant volume changes (Wessels et al., “Efficacy and Safety of Lanabecestat for Treatment of Early and Mild Alzheimer Disease: The AMARANTH and DAYBREAK-ALZ Randomized Clinical Trials,” JAMA Neurology 77:199-209 (2020), which is hereby incorporated by reference in its entirety). Observations of greater whole brain volume decrease, and greater ventricular volume increase with donanemab treatment, compared with placebo, may be interpreted in the context of protein removal rather than atrophy. Global volumetric MRI changes are typically attributed to atrophy in natural history studies of AD, but it remains unclear if they represent true atrophy in the context of rapid structural removal of protein aggregates, as seen in this study, and in another anti-amyloid therapy study (Sur et al., “BACE Inhibition Causes Rapid, Regional, and Non-progressive Volume Reduction in Alzheimer's Disease Brain,” Brain 143:3816-26 (2020), which is hereby incorporated by reference in its entirety).

ARIA-E and ARIA-H have been associated with amyloid plaque-removing treatments (Sperling et al., “Amyloid-related imaging abnormalities in amyloid-modifying therapeutic trials: Recommendations from the Alzheimer's Association Research Roundtable Workgroup,” Alzheimer's & Dementia 7:367-85 (2011); Sevigny et al., “The Antibody Aducanumab Reduces Aβ Plaques in Alzheimer's Disease,” Nature 537:50-6 (2016); Ostrowitzki et al., “Mechanism of Amyloid Removal in Patients With Alzheimer Disease Treated With Gantenerumab,” Archives of Neurology 69:198-207 (2012); Salloway et al., “Two Phase 3 Trials of Bapineuzumab in Mild-to-Moderate Alzheimer's Disease,” New England Journal of Medicine 370:322-33 (2014); Salloway et al., “A Phase 2 Multiple Ascending Dose Trial of Bapineuzumab in Mild to Moderate Alzheimer Disease,” Neurology 73:2061-70 (2009); and Sperling et al., “Amyloid-related Imaging Abnormalities in Patients with Alzheimer's Disease Treated with Bapineuzumab: A Retrospective Analysis,” Lancet Neurol. 11:241-9 (2012), which are hereby incorporated by reference in their entireties).

In the Phase 1b study, the incidence of ARIA-E was 26.1% among participants treated with donanemab, with 2 participants reporting symptomatic ARIA-E (4.3%). In this study, a similar incidence of ARIA-E was found in the donanemab group (27%), with 6.1% reporting symptomatic ARIA-E. Incidence of ARIA-E was more prevalent in ApoE4 carriers as seen in other trials of plaque targeting antibodies (Sevigny et al., “The Antibody Aducanumab Reduces Aβ Plaques in Alzheimer's Disease,” Nature 2016; 537:50-6; Ostrowitzki et al., “Mechanism of Amyloid Removal in Patients With Alzheimer Disease Treated With Gantenerumab,” Archives of Neurology 69:198-207; Salloway et al., “Two Phase 3 Trials of Bapineuzumab in Mild-to-Moderate Alzheimer's Disease,” NEJM 2014; 370:322-33 (2014); and Sperling et al., “Amyloid-related Imaging Abnormalities in Patients with Alzheimer's Disease Treated with Bapineuzumab: A Retrospective Analysis,” Lancet Neurol. 11:241-9 (2012), which is hereby incorporated by reference in its entirety). The incidence of TE-ADAs in participants treated with donanemab (approximately 90%) was similar to findings from Phase 1 (>85%).

Taken together, these results demonstrate that in participants with early symptomatic AD, treatment with donanemab resulted in amyloid plaque clearance, and a slowing of cognitive and functional decline as measured by the iADRS scale.

### Example 4: Efficacy Associated with Baseline Tau PET Patient Stratification

Donanemab was selected as an exemplary antibody for this example. The anti-N3pGlu Aβ antibody, donanemab, is found to be most efficacious in a subject with the lowest baseline flortaucipir level. The antibody may be less efficacious in subjects having high tau (>1.46 SUVr). In other words, subjects having high tau (>1.46 SUVr) may be less responsive to Aβ therapies.

Tau levels (e.g., for the purpose of stratification of the human subject suffering from AD) are determined based on an initial visual assessment of a flortaucipir scan, followed by a quantitative analysis. Visual assessment relies on a 3-tier read (tAD−, tAD+, tAD++) based on the presence of tracer uptake in specific regions of the neocortex. Quantitative analysis refers to calculation of SUVr, which represents counts within a specific target region of interest in the brain (e.g., multiblock barycentric discriminant analysis or MUBADA) when compared with a reference region (parametric estimate of reference signal intensity or PERSI). Lower SUVr values indicate less tau burden while higher SUVr values indicate greater tau burden.

A scan in the low to moderate tau group (e.g., having a SUVr from ≤1.10 to ≤1.46), as shown in Table E, is eligible for administration of anti-N3pGlu Aβ antibodies

Visual Assessment: Methods for visual assessment of human subjects are described in Fleisher et al., “Positron Emission Tomography Imaging With [¹⁸F]flortaucipir and Postmortem Assessment of Alzheimer Disease Neuropathologic Changes,” JAMA Neurol. 77(7):829-839 (2020), which is hereby incorporated by reference in its entirety. Briefly, a flortaucipir scan is negative (tAD−) if there is no increased neocortical tracer activity in any region of the brain or activity is isolated to the frontal lobe or regions of the temporal lobe that do not include the posterolateral temporal (PLT) region. Positive scans fall into two categories based on the regions of increased neocortical tracer activity. A flortaucipir scan that has neocortical tracer activity limited to the posterolateral temporal (PLT) or occipital regions is classified as tAD+.

Finally, if the flortaucipir scan shows increased tracer activity in the parietal or precuneus region or there is activity in the frontal region along with activity in PLT or occipital regions, it is classified as tAD++. Quantitative analysis is performed on all tAD+ and tAD++ scans.

Quantitative Analysis: Quantitative analysis is accomplished through an automated image processing pipeline. A previously developed neocortical target volumes of interest (VOI) (MUBADA, see Devous et al., “Test-Retest Reproducibility for the Tau PET Imaging Agent Flortaucipir F18,” J. Nucl. Med 2018; 59:937-943 (2018), which is hereby incorporated by reference in its entirety) is applied to each scan and the derived counts are normalized to a patient-specific reference region (PERSI). Other target and reference regions are also extracted through the pipeline. The PERSI reference region is a subject-specific, data-driven technique that identifies voxels with nonspecific flortaucipir uptake within an atlas-defined white matter region (see, e.g., Southekal et al, “Flortaucipir F18 Quantitation Using Parametric Estimation of Reference Signal Intensity,” J. Nucl. Med 59:944-951 (2018), which is hereby incorporated by reference in its entirety)). The MUBADA target region was developed using a statistical method to maximize separation of diagnostic groups based on image characteristics (see Devous et al, “Test-Retest Reproducibility for the Tau PET Imaging Agent Flortaucipir F18,” J. Nucl. Med. 59:937-943 (2018), which is hereby incorporated by reference in its entirety).

When applied to flortaucipir images from a large dataset of 202 subjects (55 Aβ− older cognitively normal, 43 Aβ− MCI, 54 Aβ+ MCI, 16 Aβ− AD, and 34 Aβ+ AD) the analysis yielded 2 dimensions (aka components). The first dimension (which explained 95% of the variance) provided maximal separation of groups by diagnosis and amyloid status and was converted into a VOI that is now referred to as the MUBADA VOI (see, e.g., Devous et al., “Test-Retest Reproducibility for the Tau PET Imaging Agent Flortaucipir F18,” J. Nucl. Med. 2018; 59:937-943 (2018), which is hereby incorporated by reference in its entirety)).

The MUBADA VOI ratioed to the PERSI reference region was then applied to 204 subjects and the resulting values were divided into 4 tau-burden quartiles: 1) very low; 2) low; 3) moderate; and 4) high. The cutoff SUVr values separating very low and low was 1.10; low and moderate was 1.23; moderate and high was 1.46. These values are used to screen subjects according to the algorithm described above.

Subjects with tAD+ and tAD++ scans with SUVr>1.46 were not administered anti-N3pGlu Aβ antibodies based on the hypothesis that cognitive decline in patients with high tau was driven primarily by their tauopathy and thus would not respond to anti-amyloid therapy.

TABLE E
Tau Assessment Criteria

| | Quantitative Classification |
|---|---|
| Visual Classification Criteria | Criteria (PERSI) |
| tAD− | Not measured |
| no increased neocortical activity or | |
| activity isolated to the MLT, ALT or | |
| frontal regions | |
| tAD+ | Very low tau |
| increased neocortical activity in PLT or | SUVr < 1.10 |
| occipital | Low to moderate tau |
| | 1.10 ≤ SUVr ≤ 1.46 |
| | High tau |
| | SUVr > 1.46 |
| tAD++ | Very low tau |
| increased neocortical activity in parietal | SUVr < 1.10 |
| (precuneus), or in frontal region in | Low to moderate tau |
| combination with PLT/occipital/parietal | 1.10 ≤ SUVr ≤ 1.46 |
| | High tau |
| | SUVr > 1.46 |

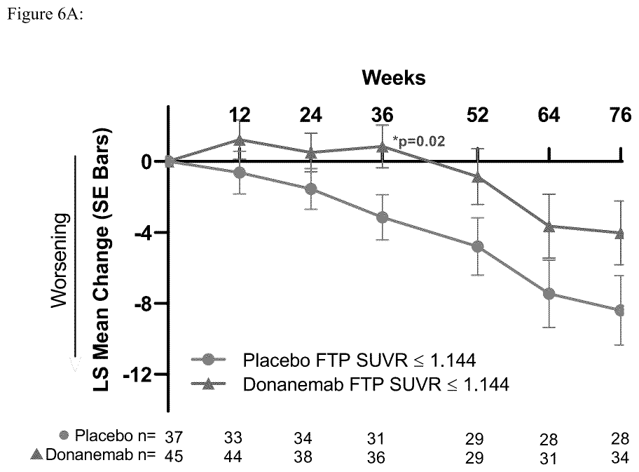

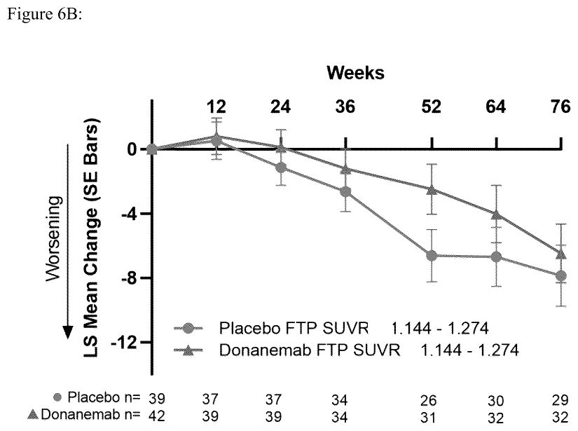

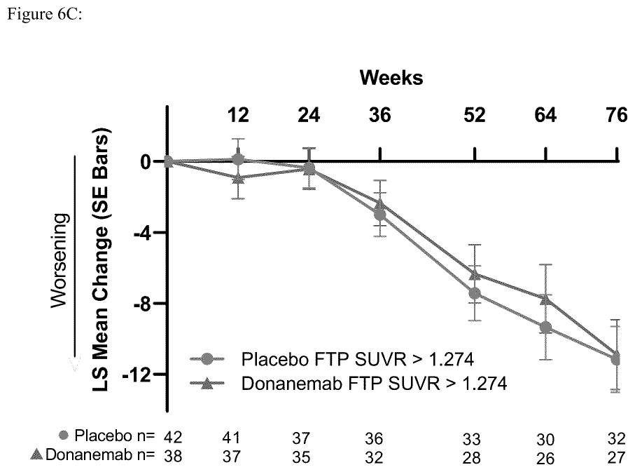

As shown in FIGS. 6A-C the anti-N3pGlu Aβ antibody, donanemab, is found to be most efficacious in the treatment sub-group with the lowest baseline flortaucipir signal. Based on the trend, it can be hypothesized that patients having high tau (>1.46 SUVr) are unlikely to be responsive to therapy.

The data demonstrate that the anti-N3pGlu Aβ antibody, donanemab, is most efficacious in human subjects having a tau level less than or equal to about 1.14 SUVr or less than or equal to about 1.27 SUVr (FIGS. 6A and 6B). The change in scale scores was not statistically significant in the donanemab treatment group compared to placebo in the graphs on the farthest right, defined by baseline tau PET SUVr values greater than 1.274 SUVr (FIG. 6C). FIGS. 6A-C show baseline tau subgroup analysis based on iADRS (FTP=Flortaucipir).

### Example 5: Comparison of Neurological Tau Burden with Cognitive Change

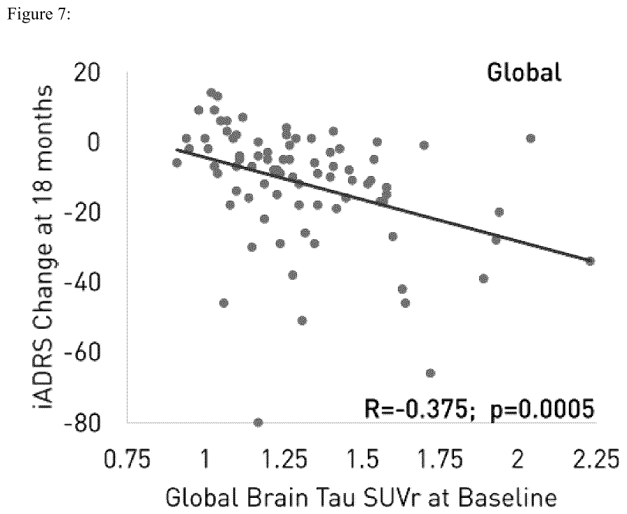

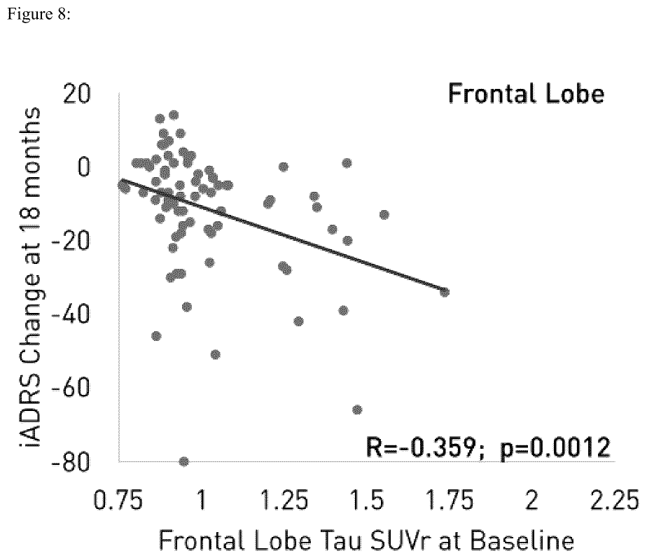

Assessment of neurological tau burden, both global and frontal lobe, in comparison to cognitive change is measured substantially as described below. Subjects are assessed for neurological tau burden, both global and frontal lobe, by flortaucipir as described herein at baseline. Additionally, at baseline, subjects are cognitively assessed under one of iADRS or CDR-SB, as known in the art. Subjects may be cognitively reassessed, for example, under one of iADRS or CDR-SB, at a given point in time thereafter, for example, at 26 weeks, 52 weeks, 78 weeks or 104 weeks. Change in cognitive assessment versus neurological tau burden may be plotted as set forth in FIGS. 5, 7, and 8. FIG. 7 shows global tau burden at baseline vs. iADRS change over 18 months. FIG. 8 shows frontal lobe tau burden at baseline vs. iADRS change over 18 months.

FIGS. 5, 7, and 8 demonstrate a lower cognitive decline associated with lower tau burden at baseline. Additionally, FIGS. 5, 7, and 8 demonstrate heterogeneity in cognitive decline among patients determined to have higher tau burden (e.g., greater then SUVR of about 1.4) at baseline.

### Example 6: Treatment of Subject Identified as Having High Tau Burden

Subjects may be determined, at baseline, to have a high tau burden according to methods as described herein, including PET imaging, including the use of flortaucipir, as well as human pTau217 assessment. Tau burden may be assessed globally, or based on a regional lobal burden (based on brain lobes), for example posterior-lateral temporal lobe, occipital lobe, parietal lobe and/or frontal lobe. Patients determined to have a high tau burden may be treated with an anti-Aβ antibody described herein and according to dose regimens as described herein.

Additionally, subjects, at baseline, may be cognitively assessed by a manner as described herein including by one of more of ADAS-Cog, iADL, CDR-SB, MMSE, APOE-4 genotyping and/or iADRS. Following treatment with an anti-Aβ Ab described herein, and according to dose regimens as described herein, subjects may be cognitively reassessed, for example at 26 weeks, 52 weeks, 78 weeks, or 104 weeks. Patients demonstrating slow, or not rapid, cognitive decline including patients determined as having a high tau burden may continue to be treated with an anti-Aβ antibody described herein.

### Example 7: Efficacy and Safety Associated with Carriers of the Allele ApolipoproteinE e4 (APOE e4)

The phase II clinical trial (NCT03367403; clinicaltrials.gov)—disclosed above in Examples 2, 3, 4, and 5—also included an examination of the efficacy and safety of the anti-N3pGlu Aβ antibody (donanemab) in the subgroup of participants that have one or two alleles of APOE e4.

This phase II clinical trial is a randomized, placebo-controlled, double-blind, multi-center Phase 2 study assessing the safety, tolerability, and efficacy of donanemab in patients with early symptomatic AD. The clinical change from baseline to 76 weeks was assessed for all enrolled patients with intermediate tau pathology levels using the Integrated AD Rating Scale (iADRS; primary endpoint), a composite tool measuring cognition and daily function, and the Clinical Dementia Rating Scale-Sum of Boxes (CDR-SB; secondary endpoint). Baseline characteristics showed that 72.5% and 74.2% of patients treated respectively with donanemab or placebo were ApoE4 carriers. Additional analysis of iADRS and key secondary endpoints were conducted focusing on this subgroup population.

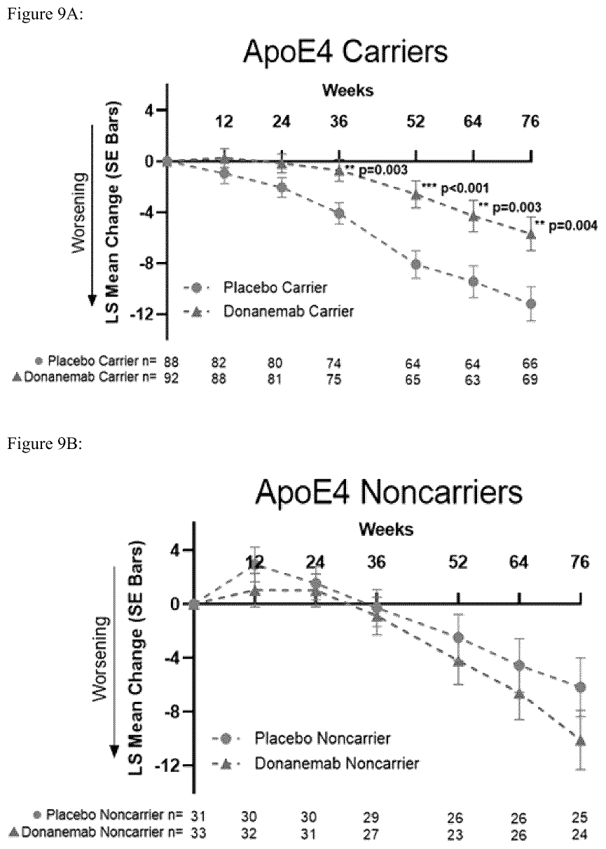

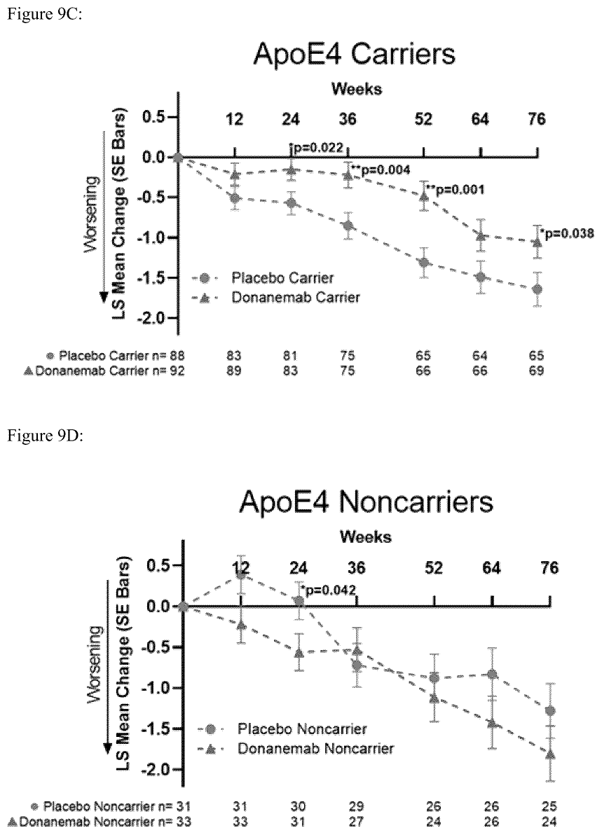

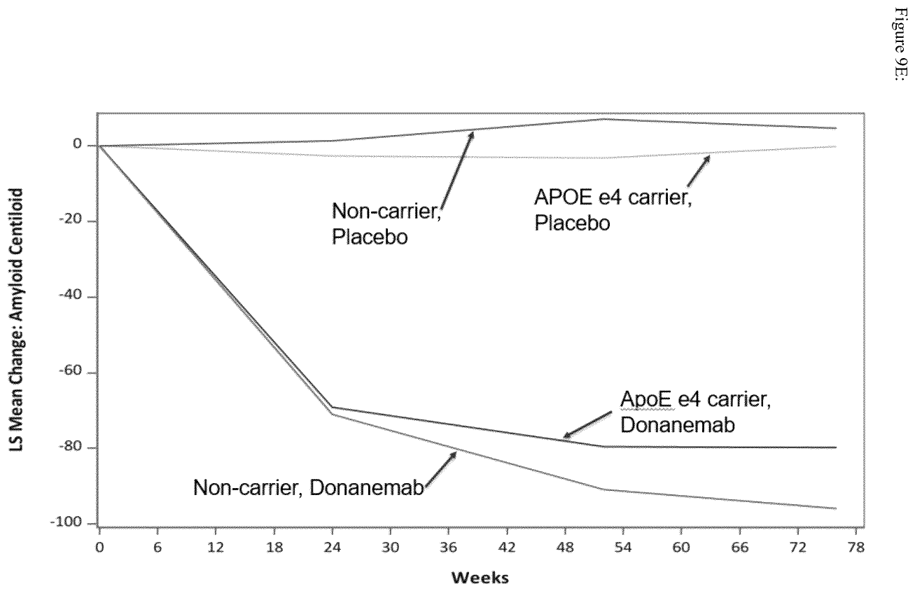

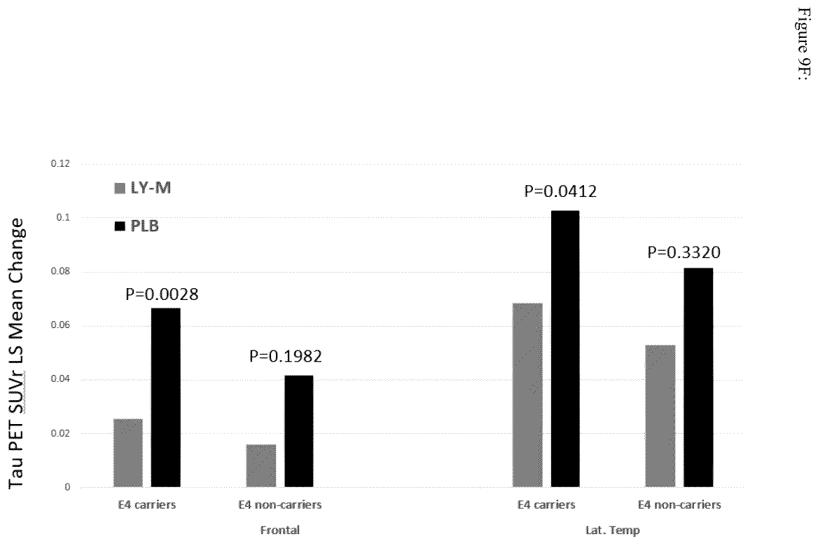

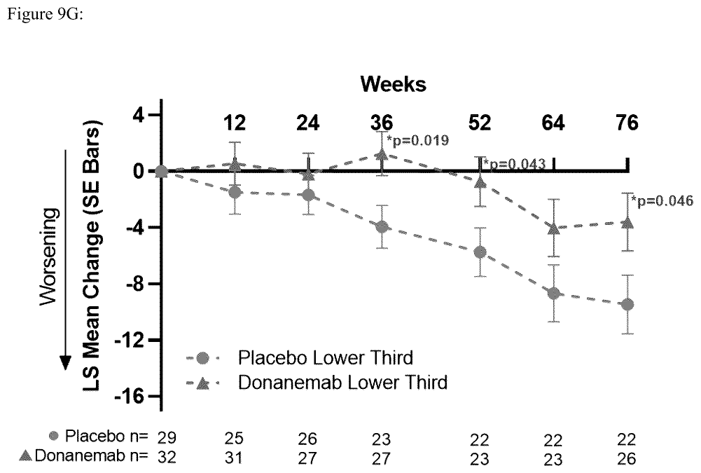

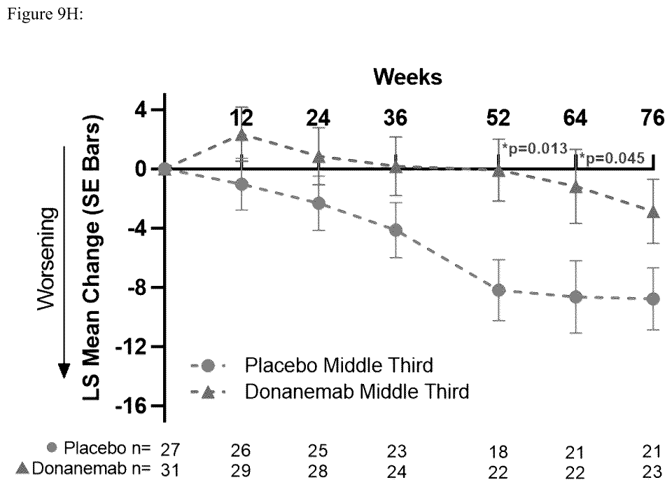

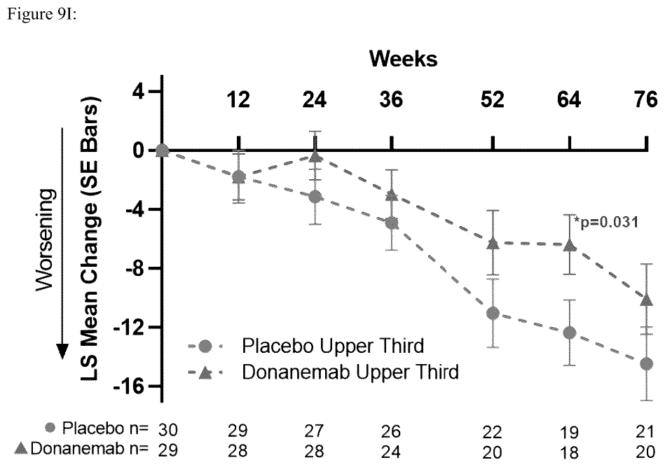

Results: Compared to placebo, donanemab treatment resulted in a 49% slowing of cognitive decline as measured with the iADRS (p=0.004) (FIG. 9A), and a 36% slowing of cognitive decline in the CDR-SB (p=0.038) in ApoE4 carriers at 76 weeks (FIG. 9C).

The donanemab treatment differences between carriers and non-carriers was significantly greater for carriers (iADRS: p=0.001, FIGS. 9A-B; CDR-SB: p=0.046, FIGS. 9C-D). Additional key secondary endpoints showed a consistent and strong efficacy of donanemab compared to placebo in ApoE4 carriers. See Table F and G below.

TABLE F
Secondary Endpoints for APOE e4 Carriers
APOE e4 Carriers

| Scale | Treatment Difference | % Slowing | P-Value |
|---|---|---|---|
| iADRS | 5.49 | 49.0 | 0.004 |
| CDR-SB | −0.59 | 36.0 | 0.038 |
| ADAS-Cog13 | −2.76 | 53.8 | 0.011 |
| ADCS-iADL | 2.61 | 44.1 | 0.030 |
| MMSE | 0.75 | 25.3 | 0.230 |

TABLE G
Secondary Endpoints for APOE e4 Non-carriers
APOE e4 Non-carriers

| Scale | Treatment Difference | % Slowing | P-Value |
|---|---|---|---|
| iADRS | −3.92 | −38.8 | 0.203 |
| CDR-SB | 0.52 | −28.9 | 0.270 |
| ADAS-Cog13 | 0.73 | −16.9 | 0.679 |
| ADCS-iADL | −3.11 | −53.9 | 0.116 |
| MMSE | 0.04 | 1.4 | 0.968 |

The safety profile for ApoE4 carriers was consistent with the overall donanemab treatment population. Donanemab's slowing of Tau PET increases was numerically larger in ApoE4 carriers dosed with donanemab than non-carriers.

Amyloid-related imaging abnormalities (ARIA) with edema or effusions, most asymptomatic, were more common in ApoE4 carriers (33.7%) than in noncarriers (8.3%). ARIA with hemosiderin deposits, like microhemorrhages, occurred in 34.5% of ApoE4 carriers receiving donanemab. Censoring carrier subjects with ARIA did not change the significance of the placebo treatment difference for iADRS (p=0.020) and CDR-SB (p=0.050).

Analyses of the study population demonstrated higher efficacy of donanemab in ApoE4 carriers than noncarriers, with significant slowing of disease progression measured in the both iADRS and CDR-SB.

FIGS. 9A-B show that Donanemab exhibits higher efficacy in APOE e4 carriers than non-carriers. FIG. 9A shows that Donanemab exhibits higher efficacy in APOE e4 carriers than non-carriers on iADRS scale. FIG. 9B shows that Donanemab exhibits higher efficacy in APOE e4 carriers than non-carriers on CDR-SB scale. FIG. 9E shows amyloid changes (centiloids) by APOE e4 status of the patients in dosed and placebo arms. FIG. 9F shows change in tau PET SUVR by APOE e4 status of the patient. The left graph shows the frontal brain lobe data for carriers (referred to in FIG. 9F as E4 carriers) and non-carriers (referred to in FIG. 9F as E4 non-carriers) for APOE e4. The right graph shows the lateral temporal brain lobe data for carriers (referred to in the Figure as E4 carriers) and non-carriers (referred to in the Figure as E4 non-carriers) for APOE e4. FIG. 9G-I show baseline tau subgroup analysis based on iADRS for APOE e4 carriers in both the donanemab treated arm and the placebo arm. The lower third shows patients with baseline flortaucipir (FTP) SUVR≤1.144 for both placebo and donanemab arms. The middle third shows patients with baseline FTP SUVR from 1.144 to 1.268 for both placebo and donanemab arms. The upper third shows patients with baseline FTP SUVR>1.268 for both placebo and donanemab arms.

Sequences (Underlined portions indicate CDRs)

SEQ ID NO: 1; Light Chain Variable Region (LCVR)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
SEQ ID NO: 2; Heavy Chain Variable Region (HCVR)
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSS
SEQ IS NO: 3; Light Chain (LC)
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
SEQ IS NO: 4; Heavy Chain (HC)
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
SEQ ID NO: 5; Light Chain Complementarity Determining Region 1 (LCDR1)
KSSQSLLYSRGKTYLN
SEQ ID NO: 6; Light Chain Complementarity Determining Region 2 (LCDR2)
AVSKLDS
SEQ ID NO: 7; Light Chain Complementarity Determining Region 3 (LCDR3)
VQGTHYPFT
SEQ ID NO: 8; Heavy Chain Complementarity Determining Region 1 (HCDR1)
GYDFTRYYIN
SEQ ID NO: 9; Heavy Chain Complementarity Determining Region 2 (HCDR2)
WINPGSGNTKYNEKFKG
SEQ ID NO: 10; Heavy Chain Complementarity Determining Region 3 (HCDR3)
EGITVY
SEQ ID NO: 11; Nucleotide Sequence for SEQ ID NO: 1; Light Chain Variable Region
(LCVR)
GATATTGTGATGACTCAGACTCCACTCTCCCTGTCCGTCACCCCTGGACAGCC
GGCCTCCATCTCCTGCAAGTCAAGTCAGAGCCTCTTATATAGTCGCGGAAAAA
CCTATTTGAATTGGCTCCTGCAGAAGCCAGGCCAATCTCCACAGCTCCTAATT
TATGCGGTGTCTAAACTGGACTCTGGGGTCCCAGACAGATTCAGCGGCAGTG
GGTCAGGCACAGATTTCACACTGAAAATCAGCAGGGTGGAGGCCGAAGATGT
TGGGGTTTATTACTGCGTGCAAGGTACACATTACCCATTCACGTTTGGCCAAG
GGACCAAGCTGGAGATCAAA
SEQ ID NO. 12; Nucleotide Sequence for SEQ ID NO: 2; Heavy Chain Variable Region
(HCVR)
CAGGTGCAGCTGGTGCAGTCTGGGGCTGAGGTGAAGAAGCCTGGGTCCTCAG
TGAAGGTTTCCTGCAAGGCATCTGGTTACGACTTCACTAGATACTATATAAAC
TGGGTGCGACAGGCCCCTGGACAAGGGCTTGAGTGGATGGGATGGATTAATC
CTGGAAGCGGTAATACTAAGTACAATGAGAAATTCAAGGGCAGAGTCACCAT
TACCGCGGACGAATCCACGAGCACAGCCTACATGGAGCTGAGCAGCCTGAGA
TCTGAGGACACGGCCGTGTATTACTGTGCGAGAGAAGGCATCACGGTCTACT
GGGGCCAAGGGACCACGGTCACCGTCTCCTCA
SEQ ID NO. 13; Nucleotide Sequence for SEQ ID NO: 3; Light Chain (LC)
GATATTGTGATGACTCAGACTCCACTCTCCCTGTCCGTCACCCCTGGACAGCC
GGCCTCCATCTCCTGCAAGTCAAGTCAGAGCCTCTTATATAGTCGCGGAAAAA
CCTATTTGAATTGGCTCCTGCAGAAGCCAGGCCAATCTCCACAGCTCCTAATT
TATGCGGTGTCTAAACTGGACTCTGGGGTCCCAGACAGATTCAGCGGCAGTG
GGTCAGGCACAGATTTCACACTGAAAATCAGCAGGGTGGAGGCCGAAGATGT
TGGGGTTTATTACTGCGTGCAAGGTACACATTACCCATTCACGTTTGGCCAAG
GGACCAAGCTGGAGATCAAACGAACTGTGGCTGCACCATCTGTCTTCATCTTC
CCGCCATCTGATGAGCAGTTGAAATCTGGAACTGCCTCTGTTGTGTGCCTGCT
GAATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGACA
GCACCTACAGCCTCAGCAGCACCCTGACGCTGAGCAAAGCAGACTACGAGAA
ACACAAAGTCTACGCCTGCGAAGTCACCCATCAGGGCCTGAGCTCGCCCGTC
ACAAAGAGCTTCAACAGGGGAGAGTGC
SEQ ID NO. 14; Nucleotide Sequence for SEQ ID NO: 4; Heavy Chain (HC)
CAGGTGCAGCTGGTGCAGTCTGGGGCTGAGGTGAAGAAGCCTGGGTCCTCAG
TGAAGGTTTCCTGCAAGGCATCTGGTTACGACTTCACTAGATACTATATAAAC
TGGGTGCGACAGGCCCCTGGACAAGGGCTTGAGTGGATGGGATGGATTAATC
CTGGAAGCGGTAATACTAAGTACAATGAGAAATTCAAGGGCAGAGTCACCAT
TACCGCGGACGAATCCACGAGCACAGCCTACATGGAGCTGAGCAGCCTGAGA
TCTGAGGACACGGCCGTGTATTACTGTGCGAGAGAAGGCATCACGGTCTACT
GGGGCCAAGGGACCACGGTCACCGTCTCCTCAGCCTCCACCAAGGGCCCATC
GGTCTTCCCGCTAGCACCCTCCTCCAAGAGCACCTCTGGGGGCACAGCGGCCC
TGGGCTGCCTGGTCAAGGACTACTTCCCCGAACCGGTGACGGTGTCGTGGAA
CTCAGGCGCCCTGACCAGCGGCGTGCACACCTTCCCGGCTGTCCTACAGTCCT
CAGGACTCTACTCCCTCAGCAGCGTGGTGACCGTGCCCTCCAGCAGCTTGGGC
ACCCAGACCTACATCTGCAACGTGAATCACAAGCCCAGCAACACCAAGGTGG
ACAAGAAAGTTGAGCCCAAATCTTGTGACAAAACTCACACATGCCCACCGTG
CCCAGCACCTGAACTCCTGGGGGGACCGTCAGTCTTCCTCTTCCCCCCAAAAC
CCAAGGACACCCTCATGATCTCCCGGACCCCTGAGGTCACATGCGTGGTGGT
GGACGTGAGCCACGAAGACCCTGAGGTCAAGTTCAACTGGTACGTGGACGGC
GTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAG
AAAACCATCTCCAAAGCCAAAGGGCAGCCCCGAGAACCACAGGTGTACACCC
TGCCCCCATCCCGGGACGAGCTGACCAAGAACCAGGTCAGCCTGACCTGCCT
GGTCAAAGGCTTCTATCCCAGCGACATCGCCGTGGAGTGGGAGAGCAATGGG
CAGCCGGAGAACAACTACAAGACCACGCCCCCCGTGCTGGACTCCGACGGCT
CCTTCTTCCTCTATAGCAAGCTCACCGTGGACAAGAGCAGGTGGCAGCAGGG
GAACGTCTTCTCATGCTCCGTGATGCATGAGGCTCTGCACAACCACTACACGC
AGAAGAGCCTCTCCCTGTCTCCGGGT

1. A method of treating or preventing a disease characterized by amyloid beta (Aβ) deposits in the brain of a human subject, the method comprising:
administering to the human subject an effective amount of an anti-Aβ antibody, wherein the human subject has been determined as having:
i) a low to moderate tau burden;
ii) a very low to moderate tau burden;
iii) a low to moderate tau burden and one or two alleles of APOE e4; or
iv) a very low to moderate tau burden and one or two alleles of APOE e4.

2.-6. (canceled)

7. The method of claim 1, wherein the human subject is administered the effective amount of the anti-Aβ antibody for a duration sufficient to treat or prevent the disease.

8. The method of claim 1, wherein the treatment or prevention of the disease causes i) reduction in Aβ deposits in the brain of the human subject and/or ii) slows cognitive or functional decline in the human subject.

9. The method of claim 8, wherein the reduction in Aβ deposits in the brain of the human subject is determined by amyloid PET brain imaging or a diagnostic that detects a biomarker for Aβ.

10. The method of claim 8, wherein the effective dose of the anti-Aβ antibody is administered to the human subject until there is about 20-100% reduction in Aβ deposits in the brain of the human subject.

11. The method of claim 10, wherein the Aβ deposits in the brain of the human subject are reduced by about 20%, about 25%, about 30%, about 35%, about 40%, about 45%, about 50%, about 75% or about 100%.

12. The method of claim 1, wherein the effective dose of the anti-Aβ antibody is administered to the human subject until the Aβ deposits in the brain of the human subject are reduced by i) about an average of about 25 centiloids to about 100 centiloids, ii) about an average of about 50 centiloids to about 100 centiloids, iii) about 100 centiloids, or iv) about 84 centiloids.

13. The method of claim 1, wherein the disease characterized by Aβ deposit in the brain of the human subject is selected from preclinical Alzheimer's disease (AD), clinical AD, prodromal AD, mild AD, moderate AD, severe AD, Down's syndrome, clinical cerebral amyloid angiopathy, or pre-clinical cerebral amyloid angiopathy.

14. The method of claim 1, wherein the human subject is an early symptomatic AD patient.

15. The method of claim 14, wherein the human subject has prodromal AD and mild dementia due to AD.

16. The method of claim 1, wherein: i) the human subject has a very low to moderate tau burden if the tau burden as measured by PET brain imaging is ≤1.46 SUVr; and ii) the human subject has a low to moderate tau burden if the tau burden as measured by PET brain imaging is from 1.10 SUVr to 1.46 SUVr.

17.-18. (canceled)

19. The method of claim 1, wherein the tau burden of the human subject is determined using PET brain imaging or a diagnostic that detects a biomarker for tau.

20. The method of claim 1, wherein the anti-Aβ antibody is an anti-N3pGlu Aβ antibody.

21. The method of claim 1, wherein administering comprises: (i) administering to the human subject one or more first doses of about 100 mg to about 700 mg of the anti-Aβ antibody, wherein each first dose is administered once about every four weeks; and (ii) about four weeks after administering the one or more first doses, administering to the subject one or more second doses of greater than about 700 mg to about 1400 mg of the anti-Aβ antibody, wherein each second dose is administered once about every 4 weeks.

22. A method for testing efficacy of an anti-Aβ antibody therapy for treating or preventing a disease characterized by amyloid beta (Aβ) deposits in the brain of a human subject, the method comprising:
(a) administering to the human subject an effective amount of an anti-Aβ antibody, wherein the human subject has been determined as having a low to moderate tau burden or a very low to moderate tau burden; and
(b) determining whether the disease has been treated or prevented.

23.-31. (canceled)

32. A method for decreasing, preventing a further increase, and/or slowing the rate of tau burden/accumulation in one or more portions of the human brain a human subject, the method comprising administering to the subject an effective amount of an anti-Aβ antibody.

33. The method of claim 32, wherein the portion of the human brain is the frontal lobe, parietal lobe, occipital lobe, temporal lobe, or posterolateral temporal lobe.

34.-37. (canceled)

38. A method of treating or preventing a disease characterized by amyloid beta (Aβ) deposits in the brain of a human subject who has been determined to have:
i) a tau burden in the temporal lobe of the brain,
ii) a tau burden in the posterolateral temporal lobe of the brain;
iii) a tau burden in the occipital lobe of the brain;
iv) a tau burden in the parietal lobe or precuneus region of the brain;
v) a tau burden in the frontal lobe of the brain;
vi) a tau burden in the posterolateral temporal lobe and/or the occipital lobe of the brain;
vii) a tau burden in the frontal lobe, and the posterolateral temporal lobe or occipital lobe of the brain
viii) a tau burden isolated to the frontal lobe; or
ix) a tau burden in regions of the temporal lobe that do not include the posterolateral temporal lobe of the brain;
the method comprising administering to the human subject an anti-Aβ antibody.

39. A method of treating or preventing a disease characterized by amyloid beta (Aβ) deposits in the brain of a human subject, the method comprising determining whether the human subject has:
i) a tau burden in the temporal lobe of the brain;
ii) a tau burden in the posterolateral temporal lobe of the brain;
iii) a tau burden in the occipital lobe of the brain;
iv) a tau burden in the parietal lobe or precuneus region of the brain;
v) a tau burden in the frontal lobe of the brain;
vi) a tau burden in the posterolateral temporal lobe and/or the occipital lobe of the brain;
vii) a tau burden in the frontal lobe, and the posterolateral temporal lobe or occipital lobe of the brain
viii) a tau burden isolated to the frontal lobe; or
ix) a tau burden in regions of the temporal lobe that do not include the posterolateral temporal lobe of the brain; and
administering an anti-Aβ antibody to the human subject.

40.-55. (canceled)

56. A method of selecting a human subject for treatment or prevention of a disease characterized by amyloid beta deposits in the brain of a human subject, the method comprising selecting the human subject based on the amount of global (overall) tau in the brain of the human subject.

57.-68. (canceled)

69. A method for determining whether to discontinue administering an anti-Aβ antibody to a human subject undergoing therapy with the anti-Aβ antibody, the method comprising determining a tau burden/accumulation in a portion of the brain of the human subject.

70. The method of claim 69, wherein determining a tau burden/accumulation comprises determining a decrease, a prevention of further increase, or a slowing in the rate of tau burden/accumulation in the portion of the brain.

71.-72. (canceled)

73. A method of treating or preventing a disease characterized by amyloid beta (Aβ) deposits in the brain of a human subject, the method comprising:
determining whether the human subject has i) low to moderate tau burden or ii) a very low to moderate tau burden; and
if the human subject has a low to moderate tau burden or a very low to moderate tau burden, administering to the human subject an effective amount of an anti-Aβ antibody.

74. A method of treating or preventing a disease characterized by amyloid beta (Aβ) deposits in the brain of a human subject, the method comprising:
determining whether the human subject has i) low to moderate tau burden and one or two alleles of APOE e4 or ii) a very low to moderate tau burden and one or two alleles of APOE e4; and
if the human subject has i) a low to moderate tau burden and one or two alleles of APOE e4 or ii) a very low to moderate tau burden and one or two alleles of APOE e4, administering to the human subject an effective amount of an anti-Aβ antibody.


SEQUENCE LISTING (14 sequences)

SEQ ID NO: 1 - protein, 112 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 2 - protein, 115 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 3 - protein, 219 aa, Artificial Sequence
  Synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 444 aa, Artificial Sequence
  Synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 16 aa, Artificial Sequence
  Synthetic construct
KSSQSLLYSRGKTYLN

SEQ ID NO: 6 - protein, 7 aa, Artificial sequence
  Synthetic construct
AVSKLDS

SEQ ID NO: 7 - protein, 9 aa, Artificial sequence
  Synthetic construct
VQGTHYPFT

SEQ ID NO: 8 - protein, 10 aa, Artificial sequence
  Synthetic construct
GYDFTRYYIN

SEQ ID NO: 9 - protein, 17 aa, Artificial sequence
  Synthetic construct
WINPGSGNTKYNEKFKG

SEQ ID NO: 10 - protein, 6 aa, Artificial Sequence
  Synthetic construct
EGITVY

SEQ ID NO: 11 - DNA, 336 bp, Artificial Sequence
  Synthetic construct DNA sequence of SEQ ID NO. 1
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaa

SEQ ID NO: 12 - DNA, 345 bp, Artificial Sequence
  Synthetic construct DNA sequence of SEQ ID NO. 2
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctca

SEQ ID NO: 13 - DNA, 657 bp, Artificial Sequence
  Synthetic construct DNA sequence of SEQ ID NO. 3
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 14 - DNA, 1332 bp, Artificial Sequence
  Synthetic construct DNA sequence of SEQ ID NO. 4
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt